(19) United States
(12) Patent Application Publication
(10) Pub. No.: US 2016/0250188 A1
(43) Pub. Date: Sep. 1, 2016

(54) METHODS FOR TREATMENT OF MUSCULAR DYSTROPHIES

(71) Applicants: Ernest D. Bush, Rockaway, NJ (US); Francois Nique, Le Perreux (FR); Catherine Jagerschmidt, Romainville (FR); Florence Sylvie Namour, Romainville (FR); Roland Blanque, Romainville (FR); Jean-Michel Lefrancois, Romainville (FR); Christophe Peixoto, Romainville (FR); Pierre Deprez, Romainville (FR); Nicolas Triballeau, Romainville (FR); Piet Tom Burt Paul Wigerinck, Mechelen (BE)

(72) Inventors: Ernest D. Bush, Rockaway, NJ (US); Francois Nique, Le Perreux (FR); Catherine Jagerschmidt, Romainville (FR); Florence Sylvie Namour, Romainville (FR); Roland Blanque, Romainville (FR); Jean-Michel Lefrancois, Romainville (FR); Christophe Peixoto, Romainville (FR); Pierre Deprez, Romainville (FR); Nicolas Triballeau, Romainville (FR); Piet Tom Burt Paul Wigerinck, Mechelen (BE)

(21) Appl. No.: 15/031,139

(22) PCT Filed: Oct. 24, 2014
(86) PCT No.: PCT/US14/62178

§ 371 Date: Apr. 21, 2016

Related U.S. Application Data
(60) Provisional application No. 61/895,832, filed on Oct. 25, 2013.

(51) Int. Cl. A61K 31/4166; A61K 9/00

(52) U.S. Cl. CPC A61K 31/4166; A61K 9/0053

(57) ABSTRACT

The present invention relates to, inter alia, treatment of muscle dystrophy (e.g., Duchenne Muscular Dystrophy), for example, using a composition, e.g., a composition comprising Compound (I), or a pharmaceutically acceptable salt, prodrug or metabolite thereof.

### CLAIMS OF PRIORITY

The present application claims priority under 35 U.S.C. §119(e) to U.S. provisional patent application, U.S. Ser. No. 61/895,832, filed on Oct. 25, 2013, which is incorporated herein by reference.

### BACKGROUND OF INVENTION

The muscular dystrophies (MD) are a group of more than 30 genetic diseases characterized by progressive weakness and degeneration of the skeletal muscles that control movement. MD weaken the musculoskeletal system and hamper locomotion. MD are caused by progressive degeneration of skeletal muscle fibres. The disease is characterized by defects in muscle proteins and the death of muscle cells and tissue.

Dystrophinopathies are a group of muscular dystrophies resulting from mutations in the dystrophin gene, located on the short arm of the X chromosome in the Xp21 region [Kunkel et al. 1985; Monaco et al. 1985; Ray et al. 1985]. Of these, Duchenne muscular dystrophy (DMD) is the most common dystrophinopathy resulting from complete absence of the dystrophin gene product, the subsarcolemmal protein dystrophin [Hoffman et al. 1987a; Koenig et al. 1987; Hoffman et al. 1988]. Its allelic variant, Becker's muscular dystrophy (BMD) is rarer with varied severity and time of presentation.

Duchenne muscular dystrophy (DMD) is a relentlessly progressive skeletal muscle disorder which, left to its natural course, results in premature death by respiratory failure by late teens, early twenties. The incidence of DMD is approximately 1 in 3300 [Jeppesen et al. 2003; CDC 2007] to 1:4700 [Dooley 2010] male births. Although a common mode of inheritance is X-linked recessive (i.e., the mother is a carrier), this disorder is associated with a high spontaneous mutation rate contributing to approximately 30% of cases [Brooks and Emery 1977; van Essen et al. 1992]. This mutation rate is estimated to be 10 times higher than for any other genetic disorder [Hoffman et al. 1992] because of the extremely large Duchenne gene size [Hoffman and Kunkel 1989]. The 2.5 million base pairs constituting the gene (a full 1% of the X chromosome) provide a large target for random mutational events. Because of this high mutation rate, eradication of the disease through genetic counseling has proven difficult.

Current therapeutic approaches to MD, e.g., DMD include the use of anabolic drugs, e.g., steroids, such as prednisolone, deflazacort, and dantrolene, which generally result in modest beneficial effects. However, treatment with anabolic drugs may also be accompanied by severe side-effects, including osteoporosis, hypertension, Cushing syndrome, weight gain, cataracts, short stature, gastrointestinal symptoms, behavioural changes, and liver damage. There is a need for new and improved treatments for MD, e.g., DMD.

### SUMMARY OF THE INVENTION

The present invention encompasses the recognition that unwanted side effects of anabolic drugs e.g., steroids, for treatment of MD, e.g., DMD, may be related to their relevant effects on androgen-sensitive tissues other than skeletal muscle, with the possibility that beneficial effects are masked by the action of the steroids on off-target sites. The present invention provides, among other things, compositions as described herein, e.g., a composition comprising the Compound (I), or a pharmaceutically acceptable salt, metabolite, or prodrug thereof, that have more specific actions on bone and skeletal muscle, e.g., as compared to anabolic drugs, and can be an alternative to treatment with anabolic drugs, e.g., steroids. The present invention provides, at least in part, methods for treating MD, e.g., DMD, and methods and kits for evaluating, identifying, and/or treating a subject, e.g., a subject suffering from or susceptible to MD, e.g., a subject suffering from or susceptible to DMD, with compositions comprising the Compound (I), or a pharmaceutically acceptable salt, metabolite, or prodrug thereof. Provided compositions and methods permit treatment of MD, e.g., DMD, with reduced associated negative side effects.

In one aspect, the invention provides a method of treating muscular dystrophy in a subject, the method comprising administering to a subject suffering from muscular dystrophy a therapeutically effective amount of the Compound (I), or a pharmaceutically acceptable salt thereof,

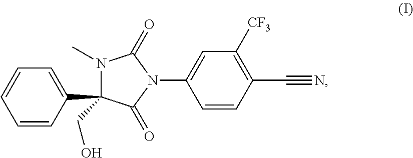 [SMILES: CN1C(=O)N(c2ccc(C#N)c(C(F)(F)F)c2)C(=O)[C@@]1(CO)c1ccccc1]

thereby treating the subject. In some embodiments, the muscular dystrophy is selected from Duchenne Muscular Dystrophy, Becker Muscular Dystrophy, Emery-Dreifuss Muscular Dystrophy, Limb-Girdle Muscular Dystrophy, Facioscapulohumeral Muscular Dystrophy, Myotonic Dystrophy, Oculopharyngeal Muscular Dystrophy, Distal Muscular Dystrophy, or congenital muscular dystrophy. In some embodiments, the muscular dystrophy is Duchenne Muscular Dystrophy.

In some embodiments, the method comprises partial or complete alleviation of an awkward manner of walking, stepping, or running; frequent falls; fatigue; difficulty with motor skills; muscle fiber deformities; pseudohypertrophy; skeletal deformities; low endurance; difficulties in standing unaided or inability to ascend staircases; loss of movement; paralysis; cardiomyopathy; development of congestive heart failure; and irregular heartbeat.

In some embodiments, the method improves (e.g., increasing, prolonging) lifespan. In some embodiments, the method comprises improving at least one symptom e.g., a symptom as described herein. In some embodiments, the symptom is fatigue, learning difficulties, intellectual disability, muscle weakness, difficulty with motor skills, difficulty walking, breathing difficulty, heart disease, cardiomyopathy, congestive heart failure, arrhythmia, scoliosis, pseudohypertrophy, muscle wasting, muscle contractures, muscle deformities, and respiratory disorders (e.g., pneumonia).

In some embodiments, the Compound (I) or pharmaceutically acceptable salt thereof is administered in multiple doses, e.g., at a predetermined interval. In some embodiments, the Compound (I) or pharmaceutically acceptable salt thereof is administered chronically (e.g., 1, 2, 3, 4, 5, 6, 7, 8, 9, or 10 times every 1, 2, 3, 4, 5, 6, days, 1, 2, 3, 4, 5, 6, 7, 8, 9 weeks, 1, 2, 3, 4, 5, 6, 7, 8, 9 months or longer) (e.g., for 1, 2, 3, 4, 5, 6, days, 1, 2, 3, 4, 5, 6, 7, 8, 9 weeks, 1, 2, 3, 4, 5, 6, 7, 8, 9 months or longer). In some embodiments, the Compound (I) or pharmaceutically acceptable salt thereof is administered once daily. In some embodiments, the Compound (I) or pharmaceutically acceptable salt thereof is administered in a single dose.

In some embodiments, the Compound (I) or a pharmaceutically acceptable salt thereof is administered at a dose of about 0.1 mg to about 1 mg (e.g., 0.1, 0.2, 0.3, 0.4, 0.5, 0.6, 0.7, 0.8, 0.9, or 1 mg) per subject. In some embodiments, the Compound (I) or a pharmaceutically acceptable salt thereof is administered at a dose of no more than 1 mg, 0.9 mg, 0.8 mg, 0.7 mg, 0.6 mg, 0.5 mg, 0.4 mg, 0.3 mg, 0.25 mg, 0.2 mg, or 0.1 mg per subject. In some embodiments, the dose is 0.1 mg per subject. In some embodiments, the dose is 0.25 mg per subject. In some embodiments, the dose is 0.5 mg per subject. In some embodiments, the dose is 1 mg per subject. In some embodiments, the dose is from e.g., about 0.2 mg to about 0.8 mg, about 0.3 mg to about 0.7 mg, or about 0.4 mg to about 0.6 mg.

In some embodiments, the Compound (I) or a pharmaceutically acceptable salt thereof is administered at a dose of about 2 μg to about 1000 μg per kilogram subject weight. In some embodiments, the Compound (I) or a pharmaceutically acceptable salt thereof is administered at a dose of no more than 1000 μg, 800 μg, 500 μg, 400 μg, 300 μg, 200 μg, 100 μg, 30 μg, 20 μg, 15 μg, 10 μg, 7 μg, or 2 μg per kilogram subject weight. In some embodiments, the dose is 2 μg per kilogram subject weight. In some embodiments, the dose is 7 μg per kilogram subject weight. In some embodiments, the dose is 15 μg per kilogram subject weight. In some embodiments, the dose is 30 μg per kilogram subject weight. In some embodiments, the dose is from about 2 μg to about 1000 μg, from about 5 μg to about 800 μg, from about 10 μg to about 500 μg, from about 10 μg to about 300 μg, from about 10 μg to about 200 μg, or from about 10 μg to about 100 μg.

In some embodiments, the Compound (I) or a pharmaceutically acceptable salt thereof is administered after meal consumption. In some embodiments, the Compound (I) or a pharmaceutically acceptable salt thereof is administered at least 60 minutes after meal consumption. In some embodiments, the Compound (I) or a pharmaceutically acceptable salt thereof is administered about 10 minutes to about 120 minutes after meal consumption. In some embodiments, the Compound (I) or a pharmaceutically acceptable salt thereof is administered about 10 minutes, about 20 minutes, about 30 minutes, about 45 minutes, about 60 minutes, about 75 minutes, about 90 minutes, about 105 minutes, or about 120 minutes after meal consumption. In some embodiments, the Compound (I) or a pharmaceutically acceptable salt thereof is administered before meal consumption. In some embodiments, the Compound (I) or a pharmaceutically acceptable salt thereof is administered about 10 minutes to about 60 minutes before meal consumption. In some embodiments, the Compound (I) or a pharmaceutically acceptable salt thereof is administered about 10 minutes, about 20 minutes, about 30 minutes, or about 45 minutes before meal consumption. In some embodiments, the Compound (I) or a pharmaceutically acceptable salt thereof is administered from 60 minutes before meal consumption to 2 hours after meal consumption.

In some embodiments, the compound converts in vivo to the Compound (II), or a pharmaceutically acceptable salt or metabolite thereof,

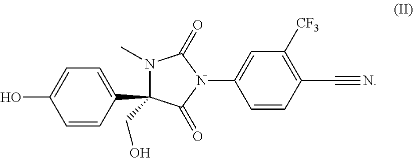 [SMILES: CN1C(=O)N(c2ccc(C#N)c(C(F)(F)F)c2)C(=O)[C@@]1(CO)c1ccc(O)cc1]

In some embodiments, the Compound (I) or pharmaceutically acceptable salt or composition thereof, is administered via oral, subcutaneous, intravenous, intramuscular, intranasal, transdermal, transmucosal, buccal, sublingual, or lung administration. In some embodiments, the Compound (I) or pharmaceutically acceptable salt or composition thereof, is administered via oral administration.

In some embodiments, the subject is human. In some embodiments, the subject is male. In some embodiments, the subject is pediatric. In some embodiments, the subject is prepubescent. In some embodiments, the subject is from the age of about 1 year to about 18 years. In some embodiments, the subject has diseased muscle (e.g., atrophy, fibrotic).

In some embodiments, the Compound (I) is substantially free of any salts or impurities. In some embodiments, the compound is in at least 95% enantiomeric excess. In some embodiments, the compound is in at least 98% enantiomeric excess. In some embodiments, the compound is in at least 99% enantiomeric excess.

In some embodiments, the levels of testosterone in the treated subject are not substantially changed as compared to levels of testosterone in the subject before treatment.

In some embodiments, the method of treatment is substantially free of any side effects e.g., obesity, behavior problems, thinner and/or weaker bones (osteoporosis); delayed puberty, stomach problems (gastroesophageal reflux or GERD), cataracts, sensitivity to infections; hypogonadism, muscle wasting and osteoporosis; cardiovascular risk (e.g., cardiovascular disease, coronary artery disease, hypertension, cardiac arrhythmias, congestive heart failure, heart attacks, sudden cardiac death); prostate cancer risks, hypogondism, and conditions pertaining to hormonal imbalances (e.g., induction of male puberty, gynecomastia, testicular atrophy, and decreased sperm production).

In some embodiments, the compound is characterized by one or both of: (a) higher activity on muscle and bones of the subject as compared to anabolic steroid treatment; and (b) lower activity on prostate of the subject as compared to anabolic steroid treatment.

In one aspect, the invention provides a pharmaceutical composition comprising the Compound (I) or a pharmaceutically acceptable salt, metabolite or prodrug thereof,

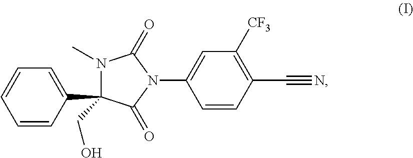 [SMILES: CN1C(=O)N(c2ccc(C#N)c(C(F)(F)F)c2)C(=O)[C@@]1(CO)c1ccccc1]

wherein the pharmaceutical composition comprises about 0.1 mg to about 1 mg of the Compound (I) or a pharmaceutically acceptable salt thereof. In some embodiments, the pharmaceutical composition comprises 0.1, 0.2, 0.25, 0.3, 0.4, or 0.5 mg of the Compound (I), or a pharmaceutically acceptable salt thereof. In some embodiments, the pharmaceutical composition comprises a pharmaceutically acceptable excipient.

In some embodiments, the pharmaceutical composition is configured in a unit dosage form. In some embodiments, the pharmaceutical composition is configured in a solid dosage form (e.g., a capsule, a tablet). In some embodiments, the solid dosage form is selected from the group consisting of tablets, capsules, sachets, powders, granules and lozenges. In some embodiments, the pharmaceutical composition is configured in a liquid dosage form.

In some embodiments, the pharmaceutical composition further comprises administering an additional therapeutic agent. In some embodiments, the additional therapeutic agent is a steroidal compound. In some embodiments, the steroidal compound is a corticosteroid, e.g., prednilosone. In some embodiments, the therapeutic agent is a non-steroidal compound.

In one aspect, the invention provides a pharmaceutical composition comprising the Compound (I) or a pharmaceutically acceptable salt thereof,

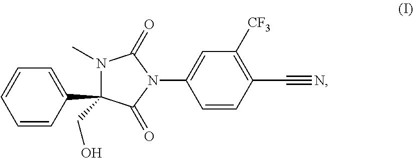 [SMILES: CN1C(=O)N(c2ccc(C#N)c(C(F)(F)F)c2)C(=O)[C@@]1(CO)c1ccccc1]

configured in a dosage form comprising no more than about 0.1 mg to about 1 mg of the Compound (I) or a pharmaceutically acceptable salt thereof per dosage form.

In one aspect, the invention provides a kit comprising the pharmaceutical composition of claim 34, and instructions for oral administration of the pharmaceutical composition to a subject in the dosage form of about 0.2 μg to about 1000 μg per kilogram subject weight.

In one aspect, the invention provides a kit comprising one or more of: Compound (I), a composition comprising Compound (I), and instructions for use in treating a subject having MD, e.g., DMD.

In one aspect, the invention provides a method of treating muscular dystrophy in a subject, the method comprising:determining whether a subject suffers from or is susceptible to muscular dystrophy; selecting the subject for treatment based on the determining; administering a therapeutically effective amount of the Compound (I) or a pharmaceutically acceptable salt thereof, thereby treating muscular dystrophy in the subject. In some embodiments, the determining comprises comparing an observed value with a reference value. In some embodiments, said subject is evaluated for a parameter described herein, e.g., as described in method of diagnosis described herein. In some embodiments, the determining comprises measuring muscle atrophy, e.g., walk test, stair climbing test.

All publications, patent applications, patents, and other references mentioned herein are incorporated by reference in their entirety.

### BRIEF DESCRIPTION OF THE DRAWINGS

The invention is herein described, by way of example only, with reference to the accompanying drawings.

FIG. 1 depicts exemplary effects of the drug treatments on contractile properties (twitch tension) of diaphragm.

FIG. 2 depicts exemplary effects of the drug treatments on contractile properties (tetanic tension) of diaphragm.

FIG. 3 depicts exemplary effects of the drug treatments on contractile properties (time to peak) of diaphragm.

FIG. 4 depicts exemplary effects of the drug treatments on contractile properties (relaxation time) of diaphragm.

FIG. 5 depicts exemplary effects of the drug treatments on contractile properties (ratio of twitch tension to tetanic tension) of diaphragm.

FIG. 6 depicts exemplary effects of the drug treatments on contractile properties of diaphragm.

FIG. 7 depicts exemplary effects of the drug treatments on contractile properties (fatigue) of diaphragm.

FIG. 8 depicts exemplary effects of the drug treatments on contractile properties (twitch tension) of EDL.

FIG. 9 depicts exemplary effects of the drug treatments on contractile properties (tetanic tension) of EDL.

FIG. 10 depicts exemplary effects of the drug treatments on contractile properties (time to peak) of EDL.

FIG. 11 depicts exemplary effects of the drug treatments on contractile properties (relaxation time) of EDL.

FIG. 12 depicts exemplary effects of the drug treatments on contractile properties (ratio of twitch tension to tetanic tension) of EDL.

FIG. 13 depicts exemplary effects of the drug treatments on contractile properties of EDL.

FIG. 14 depicts exemplary effects of the drug treatments on contractile properties (fatigue) of EDL.

FIG. 15 depicts exemplary effects of the drug treatments on mechanical threshold.

FIG. 16 depicts exemplary effects of the drug treatments on mechanical threshold.

FIG. 17 depicts exemplary effects of the drug treatments on mechanical threshold.

FIG. 18 depicts the effect of single drug treatment on total membrane ionic conductance of EDL muscle fibers of mdx mice.

FIG. 19 depicts exemplary effects of the drug treatments on levels of creatine kinase.

FIG. 20 depicts exemplary effects of the drug treatments on levels of lactate dehydrogenase.

FIG. 21 depicts exemplary effects of the drug treatments on levels of reactive oxygen species.

FIG. 22 depicts representative pictures of histology profile of diaphragm and GC muscles.

FIG. 23 depicts representative morphometric analysis following drug treatment.

FIG. 24 depicts exemplary in vivo parameters for wild-type and mdx mice at the beginning and after 4 weeks treatment with and without Compound (I), NAND, and PDN.

FIG. 25 depicts exemplary in vivo parameters of wild-type and mdx mice treated with and without Compound (I) at 0.3, 3, and 30 mg/kg for up to 12 weeks.

FIG. 26 depicts exemplary effects of treatment with Compound (I) on weight of androgen-sensitive and other potential target tissues.

FIG. 27 depicts exemplary dose- and time-dependent effects of Compound (I) on the weight of androgen-sensitive tissues and other potential target tissues.

FIG. 28 shows exemplary values of the maximal isometric twitch and tetanic tension of the diaphragm muscle from wt and mdx mice with various drug treatments.

FIG. 29 depicts exemplary isometric and eccentric contraction of isolated EDL muscles from wild-type and mdx mice treated with and without Compound (I).

FIG. 30 shows exemplary functional cellular parameters in EDL muscles in wild-type and mdx mice treated with and without Compound (I) and NAND, and PDN.

FIG. 31 depicts exemplary functional cellular parameters in EDL muscles in wild-type and mdx mice treated with and without Compound (I).

FIG. 32 depicts exemplary haematoxylin-eosin staining of the diaphragm and gastrocnemius muscles from mdx mice treated with and without Compound (I).

FIG. 33 shows exemplary effect on fibrosis markers of mdx mice treated with and without Compound (I), NAND, and PDN.

FIG. 34 depicts exemplary plasma levels of Compound (I) over 8 hours after subcutaneous delivery of the compound into mice.

FIG. 35 shows exemplary serum testosterone levels for wild-type and exercised or not-exercised mdx mice treated with and without Compound (I).

FIG. 36 depicts exemplary levels of target genes as compared to housekeeping gene GADPH after treatment with and without Compound (I).

### DETAILED DESCRIPTION

### Definitions

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

“Sample,” “tissue sample,” “subject or patient sample,” “subject or patient cell or tissue sample” or “specimen” each refers to a biological sample obtained from a tissue, e.g., a bodily fluid, of a subject or patient. The source of the tissue sample can be solid tissue as from a fresh, frozen and/or preserved organ, tissue sample, biopsy, or aspirate; blood or any blood constituents (e.g., serum, plasma); bodily fluids such as cerebral spinal fluid, whole blood, plasma and serum. The sample can include a non-cellular fraction (e.g., plasma, serum, or other non-cellular body fluid). In one embodiment, the sample is a serum sample. In other embodiments, the body fluid from which the sample is obtained from an individual comprises blood (e.g., whole blood). In certain embodiments, the blood can be further processed to obtain plasma or serum. In some embodiments, the sample contains a tissue, cells (e.g., peripheral blood mononuclear cells (PBMC)). In an embodiment the sample includes NK cells. For example, the sample can be a fine needle biopsy sample, an archival sample (e.g., an archived sample with a known diagnosis and/or treatment history), a histological section (e.g., a frozen or formalin-fixed section, e.g., after long term storage), among others (e.g., a muscle tissue section, e.g., skeletal muscle, cardiac muscle, smooth muscle). The term sample includes any material obtained and/or derived from a biological sample, including a polypeptide, and nucleic acid (e.g., genomic DNA, cDNA, RNA) purified or processed from the sample. Purification and/or processing of the sample can involve one or more of extraction, concentration, antibody isolation, sorting, concentration, fixation, addition of reagents and the like. The sample can contain compounds that are not naturally intermixed with the tissue in nature such as preservatives, anticoagulants, buffers, fixatives, nutrients, antibiotics or the like.

As used herein, “modulators” or “modulate” refers to the regulation of a protein (e.g., enzyme, receptor (e.g., androgen receptor)) by the binding of a ligand (e.g., compound, drug). Binding may be e.g., irreversible, reversible, complete or partial, at the active site or at an allosteric binding site. Modulators include antagonists, agonists, agonist-antagonists, partial antagonists, partial agonists. An “agonist” is a chemical, e.g., ligand, compound, drug, that binds to and/or upregulates some receptor (e.g., androgen receptor) of a cell and triggers a cellular response that often mimics the action of a naturally occurring substance. For example, an endogenous agonist for a particular receptor is a naturally occurring compound produced by the body that binds to and activates that receptor, e.g., endogenous agonists for the androgen receptor are androgens. An “antagonist” is a type of ligand or drug that does not provoke a biological response itself upon binding to a receptor, but blocks, dampens, or downregulates agonist-mediated responses. Antagonists generally have affinity but no efficacy for their cognate receptors, but disrupt the interaction and inhibit the function of an agonist or inverse agonist at receptors. Antagonists may be reversible or irreversible depending on the longevity of the antagonist-receptor complex. It will be appreciated by a person of skill in the art that the activity of a compound of the invention as an antagonist (complete or partial) or agonist (complete or partial) represents a continuous spectrum. Therefore, while some compounds will be clearly agonists or clearly antagonists, some compounds will exhibit both agonistic and antagonistic activity.

### Methods of Treatment

The present invention relates to, inter alia, methods for treating MD, e.g., DMD, comprising administering a composition comprising a compound as described herein, e.g., Compound (I), or a pharmaceutically acceptable salt, metabolite, or prodrug thereof. Provided compositions and methods of the present invention may, for example, increase skeletal muscle mass and/or strength, enhance protein synthesis, as well as enhance regeneration and/or metabolic efficiency.

### Muscular Dystrophy

MD are a group of more than 30 genetic diseases characterized by progressive weakness and degeneration of the skeletal muscles that control movement. MD weaken the musculoskeletal system and hamper locomotion. Some forms of MD are seen in infancy or childhood, while others may not appear until middle age or later. The disorders differ in terms of the distribution and extent of muscle weakness (some forms of MD also affect cardiac muscle), age of onset, rate of progression, and pattern of inheritance. MD are caused by progressive degeneration of skeletal muscle fibers. MD are characterized by defects in muscle proteins and the death of muscle cells and tissue. In the most severe forms, such as DMD, regeneration is exhausted and skeletal muscle is progressively replaced by fat and fibrous tissue. DMD generally causes progressive weakness in the patient and eventually death by respiratory and/or cardiac failure.

Dystrophinopathies are a group of muscular dystrophies resulting from mutations in the dystrophin gene, located on the short arm of the X chromosome in the Xp21 region [Kunkel et al. 1985; Monaco et al. 1985; Ray et al. 1985]. Of these, Duchenne muscular dystrophy (DMD) is the most common dystrophinopathy resulting from complete absence of the dystrophin gene product, the subsarcolemmal protein dystrophin [Hoffman et al. 1987a; Koenig et al. 1987; Hoffman et al. 1988]. Its allelic variant, Becker's muscular dystrophy (BMD) is rarer with varied severity and time of presentation.

The dystrophin gene is the largest human gene isolated to date. About 90% of boys have an absence of dystrophin corresponding to an “out-of-frame” mutation that disrupts normal dystrophin transcription [Gillard et al. 1989]. These mutations can cause a premature stop codon and early termination of mRNA transcription. As a result, an unstable RNA can be produced, that undergoes rapid decay, and leads to the production of nearly undetectable concentrations of truncated protein. If the mutation maintains translational reading, an “in-frame” deletion, the BMD phenotype with variably decreased amounts of abnormal molecular weight dystrophin, is present [Hoffman et al. 1988]. This reading frame hypothesis holds for about 90% of cases and is commonly used both as a diagnostic confirmation of dystrophinopathies and for the differential diagnosis of DMD and BMD. Exceptions to these two typical situations occur in approximately 10-13% of patients. [Nevo et al. 2003], [Muntoni et al. 1994]. About 60% of Duchenne and Becker patients manifest structural rearrangements of the deletion type [Kunkel 1986; den Dunnen et al. 1987]. Two deletion hotspots includes exons 45-55 and exons 2-19 [Den Dunnen et al. 1989; Oudet et al. 1992; Nobile et al. 1995]. The other 40% of patients results from small mutations (point mutations resulting in frame-shift or nonsense mutations) or duplications. Because the genetic defect is an X-linked recessive trait, dystrophinopathies are expressed primarily in boys and young men. However, girls may manifest symptoms of DMD if they also exhibit skewed X-inactivation [Lesca et al. 2003].

Dystrophin localizes to the subsarcolemmal region in skeletal and cardiac muscle and composes 0.002% of total muscle protein [Hoffman et al. 1987a]; [Hoffman et al. 1987b]. Dystrophin binds to the cytoskeletal actin and to the cytoplasmic tail of the transmembrane Dystrophin glycoprotein complex (DGC) protein alpha-dystroglycan, and thus forms a link from the cytoskeleton to the extracellular matrix. Dystrophin is organized in costamers and is present in greater amounts at myotendinous and neuromuscular junctions than in other muscle areas. In the heart it is also associated with T tubules. In smooth muscle it is discontinuous along membranes alternating with vinculin.

Muscle cell death in the muscular dystrophies (by apoptosis and necrosis) may be conditional and reflects a propensity that varies between muscles and changes with age [Rando 2001a]. The fact that adjacent muscle groups in DMD can be completely normal while others are undergoing active necrosis suggests progression is not inevitable and may be treatable. If endogenous biochemical mechanisms alter the susceptibility of a muscle cell to live or die while the genetic and biochemical defects remain constant, then pharmacological modulation of these pathways may result in successful therapies for DMD and other muscular dystrophies [Rando 2001b]. Signs and symptoms of MD include progressive muscular wasting, poor balance, drooping eyelids, atrophy, scoliosis, inability to walk, frequent falls, waddling gait, calf deformation, limited range of movement, respiratory difficulty, joint contractures, cardiomyopathy, arrhythmias, and muscle spasms. Symptoms also include fatigue, learning difficulties, intellectual disability, muscle weakness, difficult with motor skills, difficulty walking, breathing difficulty, heart disease, cardiomyopathy, congestive heart failure, arrhythmia, scoliosis, pseudohypertrophy, muscle wasting, muscle contractures, muscle deformities, and respiratory disorders (e.g., pneumonia).

Diagnosis of MD can be based on the results of a muscle biopsy, electromyography, electrocardiography, DNA analysis, and/or determination of increased creatine phosphokinase. A physician's examination and patient's medical history will aid a doctor's diagnosis in determining the type of MD a patient presents.

Existing therapeutic approaches to MD can involve steroids, e.g., prednisolone, deflazacort, and dantrolene, which result in modest beneficial effects and are typically accompanied by severe side-effects including osteoporosis, hypertension, Cushing syndrome, weight gain, cataracts, short stature, gastrointestinal symptoms, behavioural changes in the case of steroids, and liver damage.

MD includes, for example, Duchenne, Becker, Limb-girdle, Congenital, Facioscapulohumeral, Myotonic, Oculopharyngeal, Distal, and Emery-Dreifuss muscular dystrophies. In particular embodiments, certain types of MD are characterized, at least in part, by a deficiency or dysfunction of the protein dystrophin. Such muscular dystrophies include DMD and Becker Muscular Dystrophy (BMD). The various MD are discussed in further detail below.

Duchenne Muscular Dystrophy (DMD).

DMD is a relentlessly progressive skeletal muscle disorder which, left to its natural course, can result in premature death by respiratory failure by late teens, early twenties. The incidence of DMD is approximately 1 in 3300 [Jeppesen et al. 2003; CDC 2007] to 1:4700 [Dooley 2010] male births. Although a common mode of inheritance is X-linked recessive (i.e., the mother is a carrier), this disorder is associated with a high spontaneous mutation rate contributing to approximately 30% of cases [Brooks and Emery 1977; van Essen et al. 1992]. This mutation rate is estimated to be 10 times higher than for any other genetic disorder [Hoffman et al. 1992] because of the extremely large Duchenne gene size [Hoffman and Kunkel 1989]. The 2.5 million base pairs constituting the gene (a full 1% of the X chromosome) provide a large target for random mutational events. Because of this high mutation rate, eradication of the disease through genetic counseling has proven difficult.

While dystrophin deficiency can be a primary cause of DMD, multiple secondary pathways are responsible for the progression of muscle necrosis, abnormal fibrosis and failure of regeneration that results in a progressively worsening clinical status. There is evidence supporting oxidative radical damage to myofibers [Rando 2002], inflammation [Spencer and Tidball 2001; Porter et al. 2002], abnormal calcium homeostasis [Allen 2010; Millay 2009], myonuclear apoptosis [Rando 2001b; Sandri et al. 2001; Tews 2002], abnormal fibrosis and failure of regeneration [Rando 2001b; Bernasconi 1995]; [Melone 2000; Morrison 2000; Luz 2002]. This body of literature has been validated by cross sectional genome-wide approaches that allow an overall analysis of multiple defective mechanisms in DMD [Chen et al. 2000; Porter 2003]. The main symptom of DMD is muscle weakness associated with muscle wasting first with the voluntary muscles, e.g., the hips, pelvic area, thighs, shoulders, and calf muscles. Muscle weakness also occurs e.g., in the arms, neck, and other areas, but later than as in the lower half of the body. Symptoms also include an awkward manner of walking, stepping, or running (e.g., patient may walk on their forefeet, because of increased calf tonus, or toe walk to compensate for knee extensor weakness). Also, frequent falls, fatigue, difficulty with motor skills (e.g., running, hopping, jumping), increased lumbar lordosis, (e.g., leading to shortening of the hip-flexor muscles), muscle contracutures of Achilles tendon and hamstrings impairing functionality because muscle fibers shorten and fibrosis occurs in connective tissue, progressive difficulty in walking, muscle fiber deformities, pseudohypertrophy (enlarging) of tongue and calf muscles, higher risk of neurobehavioral disorders (e.g., attention deficit hyperactivity disorder, learning disorders (dyslexia), and non-progressive weaknesses in specific cognitive skills), eventual loss of ability to walk, and skeletal deformities may be associated with patients with DMD.

Symptoms usually appear in male children before the age of 6 and may be visible early in infancy. Even though symptoms do not appear until early in infancy, laboratory testing can identify children who carry the active mutation at birth. Exemplary genetic testing for early diagnosis of DMD, e.g., before onset of symptoms, are described herein and in e.g., Prior et al., Arch Pathol Lab Med. 1991 October; 115(10):984-90. Progressive proximal muscle weakness of the legs and pelvis associated with a loss of muscle mass is observed first, with the weakness eventually spreading to the arms, neck, and other areas. Early signs may include enlargement of calf and deltoid muscles (pseudohypertrophy), low endurance and difficulties in standing unaided or inability to ascend staircases. As the condition progresses, muscle tissue experiences wasting and is eventually replaced by fat and fibrotic tissue (fibrosis). By age 10, braces may be required to aid in walking, and by age 12, most patients are wheelchair dependent.

Later symptoms may include abnormal bone development that lead to skeletal deformities, including curvature of the spine. The progressive deterioration of muscle leads to loss of movement, eventually leading to paralysis. A patient with DMD may or may not present intellectual impairment. When a patient presents intellectual impairment, it typically does not progressively worsen with age. The average life expectancy for DMD patients is around 25 years of age.

DMD may be observed clinically by observing a patient's disintegrating ability to walk, for example, between the time a boy is 9 to 12 years of age. Muscle wasting begins in the legs and pelvis, progressing to the muscles of the shoulders and neck, followed by the loss of arm muscles and respiratory muscles. Calf muscle enlargement (pseudohypertrophy) can become apparent. Cardiomyopathy (e.g., dilated cardiomyopathy, DCM) is common, and the development of congestive heart failure or irregular heartbeats (arrhythmias) occurs occasionally. Children with DMD will usually tire more easily, have less overall strength than their peers, may have extremely high levels of creatine kinase, a genetic error in the Xp21 gene, and/or have an electromyography showing weakness caused by destruction of muscle tissue rather than by damage to nerves. A muscle biopsy or genetic test can confirm the absence of dystrophin.

The progression of DMD in an untreated boy can follow a predictable course. However, the disease course can be modified with aggressive pharmacological (e.g., corticosteroids) and rehabilitation treatments. The following sequence of events can eventually occur in both, treated and untreated DMD, but generally at a later age in the former. The disease is present in infancy, with muscle fiber necrosis and a high serum creatine kinase enzyme level; however, the clinical manifestations are typically not recognized until 3 years of age or later. This “therapeutic window” has been previously under-emphasized, however it lends itself to the development of early therapeutic interventions to possibly prevent or delay the onset of symptoms secondary to advance muscle degeneration. Walking might be delayed with increased falls. Gait abnormality is typically apparent at 3 to 4 years of age. Muscle weakness is usually present initially in neck flexor muscles with power being less than antigravity. As a result, the child generally needs to turn on his side when getting up from a supine position in the floor, showing the initial sign of the Gower's maneuver. Hypertrophy of calf muscles typically occurs, often being very prominent by age 3 or 4 years. Hip girdle muscles are generally affected earlier than shoulder girdle muscles. Due to weakness of the hip extensor muscles these patients tend to sway from side to side when walking, producing a waddling gait typical of the older DMD boy. Anterior hip rotation caused by muscle weakness results in increased lumbar lordosis necessary to keep the center of balance stable with shoulders lined up over hips, knees, and ankles. The preschooler can have difficulty rising from the floor, turning 45, then 90 and finally 180 degrees (depending on the degree of neck flexor weakness), and placing the hands on the floor to get up. Later, the complete Gower's′ sign may be exhibited. As muscle deterioration proceeds, climbing stairs can become difficult, requiring the use of both hands on a railing or crawling on all fours. Distal muscles of the arms and legs can show weakness as the disease progresses. Ambulation can be lost by age 10 in steroid naïve, and about 3 to 10 years later in steroid-treated DMD. Contractures of heel cords, iliotibial bands and hip flexors requiring vigorous, daily stretching may be a major problem starting as early as 4 to 5 years of age.

Accelerated deterioration in strength and balance often results from intercurrent disease or surgically induced immobilization. When ambulation is no longer possible, a wheelchair can be required. Contractures may become more pronounced in the lower extremities and soon involve the shoulders. Kyphoscoliosis may develop after ambulation is lost. Adolescent patients manifest increasing weakness and are unable to perform routine daily tasks with their arms, hands, and fingers. Pulmonary function can become compromised because of weakness of intercostal and diaphragmatic muscles and severe scoliosis, can occur later in the disease stage in non-ambulatory boys and can be a primary cause of mortality in DMD. Delaying the time to reach non-ambulatory status can have a significant impact on the development of scoliosis and respiratory function, thus in survival, which has been the case with corticosteroid treatment [Biggar et al, 2004]. The use of mechanical ventilation and good pulmonary and cardiac care have increased survival [Gomez-Merino and Bach 2002] to about 58% at age 25 (even in untreated DMD) in some countries [Eagle et al. 2002].

Boys with DMD can be at risk for cardiomyopathy, especially if they have deletion of exons 48 to 53 [Nigro et al. 1994]. Early screening for cardiomyopathy at age 5 to 6 years and then again at 10 to 12 years with an electrocardiogram (ECG) and echocardiogram can allow detection of cardiomyopathy with impaired cardiac output often before signs of heart failure are apparent. Mild degrees of cardiac compromise in DMD may occur in up to 95% of boys [Melacini et al. 1996]. Chronic heart failure may affect up to 50% [Melacini et al. 1996]. Sudden cardiac failure can occur, especially during adolescence. Subclinical or clinical cardiac insufficiency is generally present in about 90% of the DMD/BMD patients but is the cause of death in only 20% of the DMD and 50% of the BMD patients [Melacini et al. 1996; Finsterer and Stollberger 2003].

Serum creatine kinase (CK) level can be a valuable and universally used diagnostic enzyme indicator of Duchenne dystrophinopathy. CK, the muscle isoenzyme, is greatly elevated, typically from 10,000 to 30,000 times normal, early in the course of the disease. Genetic testing for DMD and BMD is widely available, especially for the deletions in the two “hot spots” of the gene. The screening of only 19 exons by multiplex PCR identifies about 98% of all deletions [Beggs et al. 1990]. Southern Blot analysis of these samples can frequently predict if the deletion, when in the rod domain, will shift the reading frame, and thus can be conclusive for DMD or BMD. The technique is very effective for the molecular diagnosis of common deletions (60% of patients). More recent technology has enabled the screening the entire dystrophin gene in search for the specific molecular defects responsible for the other 40% of DMD and BMD [Mendell et al. 2001; Dent et al. 2005]. Muscle biopsy shows fiber size variation, degenerating and regenerating fibers, clusters of smaller fibers, endomesial fibrosis, and a few scattered lymphocytes. Absence of immunoreactivity for dystrophin with monoclonal antibodies against the C-terminal, rod domain and N-terminal provide accurate diagnoses of DMD. Quantitative dystrophin analysis by immunoblot is typically more accurate for diagnosis than immunostaining, with dystrophin being less than 5% in DMD patients.

A marked elevation of plasma creatine kinase is a typical diagnostic marker of MD, e.g., DMD.

A DNA test to detect the muscle-specific isoform of the dystrophin gene mutated, muscle biopsy to reveal the absence of dystrophin protein, and prenatal tests for the presence of the most common mutations in an unborn child will indicate whether a child has the condition.

There is no cure currently for DMD. Treatment generally aimed at controlling symptoms and maximizing quality of life include corticosteroids (e.g., prednisolone, deflazacort), beta2-agonists, mild, non-jarring physical activity, physical therapy, orthopedic appliances (e.g., braces, wheelchairs), and appropriate respiratory support.

Becker Muscular Dystrophy (BMD).

BMD is a recessive X-linked form of muscular dystrophy caused by a gene mutation that results in the abnormal production of the protein dystrophin (e.g., not enough dystrophin or faulty dystrophin). BMD is a less severe variant of DMD in that the symptoms appear later and progress more slowly. BMD affects only 1 in 30,000 males, with symptoms usually appearing between the ages of 2 and 16 and occasionally appearing as late as age 25. The condition can cause heart problems and the severity will vary. BMD patients usually survive into old age.

Congenital Muscular Dystrophy.

Congenital muscular dystrophies present in patients at birth or in the first few months of life, progress slowly, and affect both males and females. Symptoms include general muscle weakness and possible joint deformities. The two identified forms, Fukuyama and congenital muscular dystrophy with myosin deficiency, cause muscle weakness along with severe and early contractures (e.g., shortening or shrinking of muscles, joint problems). Fukuyama congenital muscular dystrophy causes abnormalities in the brain (e.g., seizures). Congenital MD typically progresses slowly and generally results in shortened life span. Resultant muscle degeneration may be mild or severe, may be restricted to skeletal muscle or paired with effects on the brain and other organ systems. Some forms of congenital MD are caused by defects in proteins that relate to the dystrophin-gycloprotein complex and to the connections between muscle cells and their surrounding cellular structure.

Distal Muscular Dystrophy.

Distal muscular dystrophy is a rare form of muscular dystrophy that affects both adult men and women, typically from about 20 to 60 years of age, causing weakness and wasting of distal muscles (e.g., forearms, hands, lower legs, feet). Distal muscular dystrophy is less severe, progresses more slowly, and affects fewer muscles than other forms of muscular dystrophy. Distal MD is typically not life-threatening.

Emery-Dreifuss Muscular Dystrophy.

Emery-Dreifuss is a rare form of muscular dystrophy appearing from childhood to early teenage years and affects only males. Muscle shortening (contractures) can occur early in the disease, progressing slowly with muscle weakness spreading to the limb-girdle muscles, e.g., chest and pelvic muscles later. Emery-Dreifuss causes muscle weakness and wasting in the shoulders, upper arms, and lower legs, but causes less severe muscle weakness than other forms of muscular dystrophy. Cardiac conduction defects and arrhythmias can also effect patients, which if left untreated increase the risk of stroke and sudden death.

Three subtypes of Emery-Dreifuss MD exist, usually distinguishable by their pattern of inheritance: X-linked, autosomal dominant, and autosomal recessive. The X-linked form is the most common. The disease can be caused by mutations in the LMNA gene, also known as the EMD gene. Both genes encode for proteins of the nuclear envelope.

Facioscapulohumeral Muscular Dystrophy (FSHD).

FSHD is a form of muscular dystrophy that effects the muscles that move the face, shoulder blade, chest, upper arm bone, arms, and legs. FSHD usually begins in the teenage years to early adulthood and can affect both males and females. The condition generally progresses slowly, with short periods of rapid muscle deterioration and weakness. The severity can range from very mild to completely disabling, often affecting walking, chewing, swallowing, and causing speaking problems. Most FSHD patients live a normal life span, with about half retaining the ability to walk throughout their life.

Limb-Girdle Muscular Dystrophy (LGMD).

LGMD cause progressive weakness that begins in the hips and moves to the shoulders, arms, and legs. Walking can become difficult or impossible within 20 years, and patients with LGMD typically live to middle age to late adulthood. Many forms of LGMD have been identified, showing different patterns of inheritance, e.g., autosomal recessive, autosomal dominant. The recessive forms have been associated with defects of proteins of the dystrophin-glycoprotein complex. Patients that suffer from LGMD can lead a normal life with some assistance, but in extreme cases can die from e.g., cardiopulmonary complications.

Myotonic Muscular Dystrophy.

Mytonic muscular dystrophy is also known as MMD or Steinert's disease, and is the most common form of muscular dystrophy in adults. Myotonic muscular dystrophy affects both men and women and usually present any time from early childhood to adulthood. It will sometimes appear in newborns (e.g., congenital MMD). A symptom of myotonic muscular dystrophy is prolonged spasm or stiffening of muscles (or myotonia), which can be worse in cold temperatures. The condition also affects the central nervous system, heart, gastrointestinal tract, eyes, and hormone-producing glands. MMD does not usually restrict daily living, although patients with myotonic muscular dystrophy have a decreased life expectancy. Mytonic dystrophy varies in severity and its manifestations and affects many body systems in addition to skeletal muscles, e.g., the heart, endocrine organs, eyes, and the gastrointestinal tract. MMD is typified by prolonged muscle spasms, cataracts, cardiac abnormalities, and endocrine disturbances. Individuals with MMD typically have long, thin faces, drooping eyelids, and a swan-like neck.

Steinert disease is the most common form of MD and results from the expansion of a short repeat in the DNA sequence of the myotonic dystrophy protein kinase gene. Myotonic MD type 2 is much rarer and is a result of the expansion of the CCTG repeat in the zinc finger protein 9 gene, which may interfere with the production of important muscle proteins.

Oculopharyngeal Muscular Dystrophy.

Oculopharyngeal muscular dystrophy can appear both in men and women in their 40s, 50s, and 60s, and causes weakness in the eye and face muscles. Oculopharyngeal muscular dystrophy can lead to difficulty swallowing, with weakness in the pelvic and shoulder muscles generally occurring later. Choking and recurrent pneumonia can occur in patients with this condition.

Methods of the invention may include administering, for example, Compound (I), or a pharmaceutically acceptable salt, metabolite, or prodrug thereof, or a composition comprising Compound (I), or a pharmaceutically acceptable salt, metabolite, or prodrug thereof, that may show good absorption, good half-life, good solubility, good bioavailability, low protein binding affinity, reduced drug-drug interaction, good metabolic stability, and reduced side effects, e.g., less off-target effects, for example, as compared to an alternative therapy, e.g., anabolic drug therapy. In an aspect, compounds of the present invention exhibit significant improvements in pharmacological properties, e.g., improved bioavailability, improved efficacy, reduction of side effects. Where a compound of the present invention exhibits any one or more of these improvements, it would be expected that such a compound will confer advantages in the potential uses of the compound. For example, where a provided compound exhibits improved bioavailability, it would be expected that the compound could be administered at a lower dose, thus reducing the occurrence of possible undesired side effects.

Provided methods may be used to effectively treat individuals suffering from or susceptible to MD, e.g., DMD. The term, “treat” or “treatment,” as used herein, refers to the application or administration of a compound and/or composition, alone or in combination with, one or more additional compounds to a subject, e.g., a subject, or application or administration of the compound and/or composition to an isolated tissue or cell, e.g., cell line, from a subject, e.g., a subject, who has a disorder (e.g., a disorder as described herein), a symptom of a disorder, or a predisposition toward a disorder, with the purpose to cure, heal, alleviate, relieve, alter, remedy, ameliorate, improve or affect the disorder, one or more symptoms of the disorder or the predisposition toward the disorder (e.g., to minimize at least one symptom of the disorder or to delay onset of at least one symptom of the disorder), and/or lessening of the severity or frequency of one or more symptoms of the disease. Exemplary symptoms of MD include, but are not limited to, muscle degeneration, muscle weakness, muscle wasting, awkward manner of walking, stepping, or running, frequent falls, fatigue, difficulty with motor skills, muscle fiber deformities, pseudohypertrophy, skeletal deformities, low endurance, difficulty in standing unaided or inability to ascend staircases, loss of movement, paralysis, cardiomyopathy, and development of congestive heart failure or irregular heartbeats.

It will be appreciated that symptoms of MD may be measured by any available method. For example, muscle atrophy may be measured by percent functional activity remaining, as determined by e.g., an ambulation test (e.g., duration walk test, distance walk test), timed function tests (e.g., time to stand from supine position, time to run/walk 10 meters, time to ascend or descend stairs), myometer (e.g., upper, lower extremity myometry), health-related quality of life (e.g., physical, emotional, social function), energy expenditure (e.g., active versus resting heart rate divided by walking velocity), respiratory function, or electrical impedance myography (EIM). EIM is a non-invasive technique that can measure the health of a muscle and track its changes over time by measuring electrical impedance of individual muscles as a diagnostic tool.

In some embodiments, treatment refers to partial or complete alleviation, amelioration, relief, inhibition, delaying onset, reducing severity and/or incidence of muscle degeneration, muscle weakness, or muscle wasting. In some embodiments, muscle degeneration, muscle weakness, or muscle wasting is characterized by awkward manner of walking, stepping, or running, frequent falls, fatigue, difficulty with motor skills, muscle fiber deformities, pseudohypertrophy, skeletal deformities, low endurance, difficulties in standing unaided or inability to ascent staircases, loss of movement, paralysis, cardiomyopathy, and development of congestive heart failure or irregular heartbeats. In some embodiments, treatment refers to partial or complete alleviation, amelioration, relief, inhibition, delaying onset, reducing severity and/or incidence of awkward manner of walking, stepping, or running, frequent falls, fatigue, difficulty with motor skills, muscle fiber deformities, pseudohypertrophy, skeletal deformities, low endurance, difficulties in standing unaided or inability to ascent staircases, loss of movement, paralysis, cardiomyopathy, and development of congestive heart failure or irregular heartbeats. In some embodiments, treatment refers to improving (e.g., increasing, prolonging) lifespan.

In some embodiments, provided methods improve one or more symptoms of MD, e.g., DMD, in a subject. For example, a compound of the present invention may be administered for a time and in an amount sufficient to reduce fatigue, learning difficulties, intellectual disability, muscle weakness, difficulty with motor skills, difficulty walking, breathing difficulty, heart disease, cardiomyopathy, congestive heart failure, arrhythmia, scoliosis, pseudohypertrophy, muscle wasting, muscle contractures, muscle deformities, and respiratory disorders (e.g., pneumonia) associated with MD, e.g., DMD, thereby improving the symptom(s) of MD, e.g., DMD. Such improvements in symptoms may be determined in the subject by one or more methods described herein.

In certain embodiments, a symptom as described herein, is decreased by about 5%, 10%, 15%, 20%, 25%, 30%, 35%, 40%, 45%, 50%, 55%, 60%, 65%, 70%, 75%, 80%, 85%, 90%, 100% or more in a subject as compared to a control, e.g., reference or historical sample, untreated subject or subject treated with placebo.

In some embodiments, treatment refers to increased survival (e.g., survival time). For example, treatment can result in an increased life expectancy of a patient. In some embodiments, treatment according to the present invention results in an increase life expectancy of a patient by more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 200% or more, as compared to the average life expectancy of one or more control individuals with similar disease without treatment. In some embodiments, treatment according to the present invention results in an increased life expectancy of a patient by more than about 6 months, about 7 months, about 8 months, about 9 months, about 10 months, about 11 months, about 12 months, about 2 years, about 3 years, about 4 years, about 5 years, about 6 years, about 7 years, about 8 years, about 9 years, about 10 years or more, as compared to the average life expectancy of one or more control individuals with similar disease without treatment. In some embodiments, treatment according to the present invention results in long term survival of a patient. As used herein, the term “long term survival” refers to a survival time or life expectancy longer than about 40 years, 45 years, 50 years, 55 years, 60 years, or longer.

### Target Tissues

As used herein, the term “target tissues” refers to any tissue that is affected by MD, e.g., DMD. Exemplary target tissues include bone, skeletal muscle (e.g., diseased skeletal muscle), voluntary muscles (e.g., hips, pelvic area, thighs), muscles of the upper body (e.g., arms, neck, shoulders), muscles of the lower body (e.g., hip-flexor muscles, calf muscles, Achilles tendon, hamstrings). In some embodiments, a target tissue is cardiac muscle. In some embodiments, a target tissue is diaphragm. In some embodiments, target tissues include those tissues in which there is an absence or abnormal presence of dystrophin protein (e.g., deficiency or dysfunction in dystrophin protein). Target tissues may, for example, refer to skeletal muscle, e.g., diseased skeletal muscle. In some embodiments, the methods of the present invention affect skeletal muscle. Skeletal muscle is one of three major muscle types (skeletal, cardiac, and smooth). Skeletal muscle is a form of striated muscle tissue and is controlled by the somatic nervous system (it is voluntarily controlled). Skeletal muscles are attached to bones by tendons, which are bundles of collagen fibers.

“Off-target tissues” refer to any tissue that is not a target tissue, e.g., the heart, sex-related organs, organs related to reproduction (e.g., prostate).

In some embodiments, the methods as described herein are delivered preferentially to one or more target tissues. In some embodiments, the compounds described herein (e.g., Compound (I)) bind to a target tissue with e.g., 2, 3, 4, 5, 6, 7, 8, 9, 10 or more fold higher affinity than they bind off-target tissues. In some embodiments, the compounds described herein (e.g., Compound (I)) bind to a target tissue with e.g., 100%, 150%, 200% 250%, 300% or more higher affinity than binding to off-target tissues.

### Side Effects

Adverse side effects that may result from treatment of subjects with MD, e.g., DMD, with existing therapies, e.g., anabolic drugs, include obesity, behavior problems, thinner and/or weaker bones (osteoporosis), delayed puberty, stomach problems (gastroesophageal reflux or GERD), cataracts, and sensitivity to infections. Provided compositions and methods can act, e.g., exert biological effect, e.g., modulate the androgen receptor, in target tissues, e.g., specifically, decreasing or reducing adverse side effects. Androgens, e.g., testosterone and dihydrotestosterone, control a broad spectrum of physiological processes through intracellular androgen receptors. Alteration of the circulating levels of androgens or androgen receptor modulation, e.g., mutation or change in the dynamic intracellular androgen receptor complex, can lead to disorders such as hypogonadism, muscle wasting and osteoporosis. Therefore, treatment with testosterone is associated with potential cardiovascular risk (e.g., cardiovascular disease, coronary artery disease, hypertension, cardiac arrhythmias, congestive heart failure, heart attacks, sudden cardiac death) and prostate cancer risks.

Anabolic steroids e.g., nandrolone, oxandrolone, are steroidal drugs that have similar effects to testosterone in the body. Anabolic steroids can produce effects on androgen-sensitive tissues other than the skeletal muscle that mask the beneficial effects of the steroids in target tissues. Undesired side effects from anabolic steroids may be related to action of the steroids on off-target sites (sites other than the target tissues) and include cardiovascular risk, prostate cancer risks, and hypogondism. Side effects also include conditions pertaining to hormonal imbalances (e.g., induction of male puberty, gynecomastia, testicular atrophy, and decreased sperm production), harmful changes in cholesterol levels (e.g., increased low-density lipoprotein and decreased high-density lipoprotein), acne, high blood pressure, liver damage, and dangerous changes in the structure of the left ventricle of the heart. Side effects will vary depending on the length of use, can damage the immune system, elevate blood pressure (e.g., especially in those with existing hypertension), produce premature baldness, cause liver damage, reduce sexual function, and result in temporary infertility. Particularly in adolescents, side effects may include premature stop of the lengthening of bones (premature epiphyseal fusion through increased levels of estrogen metabolites), stunted growth, accelerated bone maturation, increased frequency and duration of erections, and premature sexual development. Psychiatric side effects include poorer attitudes related to health, aggression, violence, mania, psychosis, mood disorders, and suicide.

Provided methods may result in levels of testosterone in the treated subject that are not substantially changed as compared to levels of testosterone present in the subject before treatment. In some embodiments, provided methods result in testosterone levels in the treated subject that are within a normal reference range for a non-treated subject of the same sex and age. In some embodiments, the method of treatment is substantially free of any side effects in the subject.

### Subjects

A subject to be treated by provided compositions and/or methods suffer from or are susceptible to an MD, such as Becker muscular dystrophy, congenital muscular dystrophy, Duchenne muscular dystrophy, distal muscular dystrophy, Emery-Dreifuss muscular dystrophy, facioscapulohumeral muscular dystrophy, limb-girdle muscular dystrophy, myotonic muscular dystrophy, or oculopharyngeal muscular dystrophy. A subject to be treated can have diseased muscle (e.g., atrophy, fibrotic), for example, as determined by a muscle biopsy or other diagnostic method. As used herein, the term “subject” is intended to include human and non-human animals, e.g., vertebrates, large animals, and primates. In certain embodiments, the subject is a mammalian subject, and in particular embodiments, the subject is a human subject. Although applications with humans are clearly foreseen, veterinary applications, e.g., with non-human animals, are also envisaged herein. The term “non-human animals” of the invention includes all vertebrates, e.g., non-mammals (such as chickens, amphibians, reptiles) and mammals, such as non-human primates, domesticated and/or agriculturally useful animals, e.g., sheep, dog, cat, cow, pig, among others.

In some embodiments, the subject is male. In some embodiments, the subject is pediatric, e.g., from birth to about age 21 years. For example, the subject may be 21 years of age or younger, e.g., 18 years, 16 years, 14 years, 12 years, 10 years, 8 years, 6 years, 4 years, 2 years, 1 year of age or younger. In some embodiments, the subject is prepubescent, e.g., in males, puberty typically begins around age 11 or 12. Typically, puberty in males is complete by ages 16 to 17. For example, the subject may be a male between ages 10 and 18 years, between ages 11 and 17 years, between ages 12 and 16 years, between ages 13 and 15 years.

Exemplary human subjects include a human subject having a disorder, e.g., a disorder described herein, or a normal subject.

As discussed above, MD, e.g., DMD refers to a group of muscle diseases having defects in muscle membrane or muscle proteins characterized, in part, by ongoing muscle degeneration and regeneration leading to progressive muscle weakness, increased susceptibility to muscle damage, and degeneration and death of muscle cells and tissues. The determination as to whether a subject has MD, as well as the determination of a particular type of MD, can be made by any measure accepted and utilized by those skilled in the art. For example, diagnosis of subjects can include a targeted medical history and examination, biochemical assessment, muscle biopsy, and/or genetic testing.

A subject's medical history may be used to diagnose MD, e.g., DMD. For example, subjects with DMD are typically symptomatic before the age of 5 years, and experience difficulty running, jumping, and climbing steps. Proximal weakness causes individuals to use their arms in rising from the floor (i.e. Gowers' sign). Independent ambulation is often lost by 14 years of age, with subsequent deterioration in respiratory function and development of contractures and scoliosis. Subjects commonly suffer static cognitive impairment. Approximately one third of boys with DMD develop cardiomyopathy by 14 years of age, and most all do after 18 years. Congestive heart failure and arrhythmias are common in end-stage DMD. Most young men with DMD die in their late teens or early twenties from respiratory insufficiency or cardiac failure.

Biochemical assessments, such as, for example, measurement of enzymatic activity and expression levels, e.g., serum creatine kinase levels, lactate dehydrogenase levels, may be used to diagnose a subject having muscular dystrophy (e.g., DMD). Increased serum creatine kinase levels indicate increased muscle damage. The present invention provides treatment of subjects having muscular dystrophy with high or elevated serum creatine kinase levels. In certain embodiments, a human subject suitable for treatment using the present methods is a subject having MD, e.g., DMD with high or elevated serum creatine kinase levels, particularly when the subject has a condition as described herein. Increased serum lactate dehydrogenase levels indicate increased metabolic distress. The present invention provides treatment of subjects having MD, e.g., DMD with high or elevated lactate dehydrogenase levels. In certain embodiments, a human subject suitable for treatment using the present methods is a subject having MD, e.g., DMD with high or elevated serum lactate dehydrogenase levels, particularly when the subject has a condition as described herein. In some embodiments, the serum creatine kinase, as measured in units of enzymatic activity per liter (U/L), is greater than 5000, 6000, 7000, 8000, 9000, 10000, or 11000. In some embodiments, the serum creatine kinase, as measured in units of enzymatic activity per liter (U/L), is between 5000 to 25000, 7500 to 20000, or 10000 to 20000. In some embodiments, the serum creatine kinase levels are 2, 3, 4, 5, 6, 7, 8, 9, 10, 20, 30, 40, 50, 60, 70, 80, 90, 100, or more times higher than the serum creatine kinase levels at birth.

Muscle biopsy may also be used to diagnose a subject as having MD, e.g., DMD. For example, muscle biopsy from DMD patients shows degeneration, regeneration, and variability of fiber size with replacement of muscle by fat and connective tissue. The present invention provides methods for treatment of MD, e.g., DMD in a subject with reduced or low muscle dystrophin levels.

Genetic testing may also be employed to diagnose a subject as having muscular dystrophy. Techniques used in genetic testing include the polymerase chain reaction (PCR), Southern blotting, mutation scanning, and/or sequence analysis. Deletions in the dystrophin gene are detected in 65% of DMD patients and 85% of BMD patients. Quantitative assays of dystrophin may be used to predict phenotype (e.g., DMD patients have less than 5% of the normal quantity of dystrophin, BMD patients have at least 20% normal dystrophin levels). Analysis of genes involved in the control of muscle mass, (e.g., a marker of muscle regeneration or muscle growth, e.g., myogenin, IGF-1, follistatin); modulators of muscle metabolism and of mechanotransduction signaling, (e.g., peroxisome proliferator receptor γ-coactivator (PGC)-1α) can be used to diagnose a subject as having MD, e.g., DMD. For example, to perform genetic testing, a single routine blood sample may be collected which can be analyzed for a mutation in the dystrophin DNA. The test can also determine the type of mutation (e.g., deletion, duplication, insertion, missense, nonsense) and determine its location within the dystrophin gene. Deletions and duplications in the dystrophin DNA may be first tested for, followed by a second test involving gene sequencing and sequence analysis, which can determine e.g., gene changes, insertions, missense, nonsense mutations.

Biochemical assessments, such as, for example, metabolite profiling or measurement of metabolite levels, e.g., testosterone levels (e.g., free, total), may be used to determine off-target effects of a compound, composition, or method of treatment of a subject having MD (e.g., DMD). Testosterone levels may be determined from e.g., a blood test, saliva test, urine test, and testosterone levels can be analyzed e.g., through an electrochemiluminescent immunoassay (ECLIA), liquid chromatography-mass spectrometry (LC/MS) method.

In some embodiments, the methods as described herein result in subjects with increased levels of testosterone in target tissues, (e.g., skeletal muscle, e.g., diseased skeletal muscle), relative to off-target tissues, (e.g., prostate), as compared to untreated subjects. The present invention provides, among other things, treatment of subjects having muscular dystrophy, which treatment results in normal levels, e.g., physiological levels of testosterone in off-target tissues. In some embodiments, the levels of testosterone in the treated subject are not substantially changed as compared to levels of testosterone present in the subject before treatment. In some embodiments, provided compositions and methods are characterized by one or both of: a) higher activity on the muscle and bones of the subject as compared to anabolic drug, e.g., steroid treatment, b) lower activity on prostate of the subject as compared to anabolic drug, e.g., steroid treatment.

### Patient Selection and Monitoring

Provided herein are compositions and methods for treating MD, e.g., DMD, in a subject. Further provided are methods of determining whether a subject suffers from MD, e.g., DMD; selecting the subject for treatment based on the determining (e.g., measuring muscle wasting, muscle fibrosis); administering an effective amount of the Compound (I) or a pharmaceutically acceptable salt thereof, thereby treating MD, e.g., DMD in the subject. Also described herein are methods of predicting a subject who is at risk of developing MD, e.g., DMD (e.g., by biochemical assessments, e.g., measurement of enzymatic activity and expression levels, e.g., serum creatine kinase levels, lactate dehydrogenase levels; by genetic testing, e.g., quantitative assays of dystrophin, myogenin, ICF-1, follistatin, and or (PGC)-1α).

In some embodiments, a subject is selected for treatment based on a determination that a subject has MD, e.g., DMD as diagnosed by e.g., medical history, genetic testing, muscle biopsy, biochemical assessments of a subject.

In some embodiments, the subject has previously been treated for MD, e.g., DMD with one or more of steroids, albuterol, angiotensin-converting enzyme inhibitors, beta-blockers, diuretics, proton pump inhibitors, amino acids, carnitine, coenzyme Q10, creatine, fish oil, green tea extract, or vitamin E.

In one aspect, the present invention is a method of evaluating treatment of MD, e.g., DMD in a subject, comprising: acquiring a MD, e.g., DMD status value in the subject; responsive to the acquired MD, e.g., DMD value, administering a pharmaceutical composition comprising Compound (I) to the subject; detecting a change in the MD, e.g., DMD status value in the subject at one or more predetermined time intervals; thereby evaluating the treatment of MD, e.g., DMD in the subject. In some embodiments, the method comprises performing one or more of the following: continuing administration of the pharmaceutical composition at the same schedule, time course, or dosing; administering an altered dose of the pharmaceutical composition; altering the schedule or time course of administration of the pharmaceutical composition; or administering alternative therapy, thereby treating MD, e.g., DMD, in the subject.

### Compounds

Compound (I) (also known as GLPG0492, G100192) is a compound that can affect the activity of, e.g., modulate, the androgen receptor (AR). The active agent is the Compound (I):

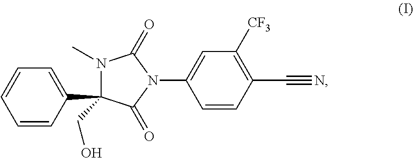 [SMILES: CN1C(=O)N(c2ccc(C#N)c(C(F)(F)F)c2)C(=O)[C@@]1(CO)c1ccccc1]

or a pharmaceutically acceptable salt, metabolite, or prodrug thereof, for example, as disclosed in WO 2010/029119. In some embodiments, the active agent is a prodrug of Compound (I). In some embodiments, the active agent is a metabolite of the Compound (I). In some embodiments, Compound (I) is metabolized, e.g., oxidized in vivo, into the Compound (II):

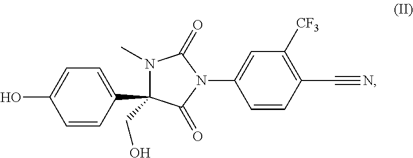 [SMILES: CN1C(=O)N(c2ccc(C#N)c(C(F)(F)F)c2)C(=O)[C@@]1(CO)c1ccc(O)cc1]

or a pharmaceutically acceptable salt thereof. In some embodiments, the active agent is Compound (II). In some embodiments, the active agent is a prodrug of Compound (II).

As used herein, the term “metabolite” refers to a compound that has been processed, e.g., in the body of a subject, into a drug. Metabolites are the intermediates and products of metabolism, e.g., formed as a part of the natural biochemical process of degradation and elimination of compounds. In an embodiment, the processing comprises the breaking or formation of a bond, e.g., a covalent bond. In some embodiments, the processing comprises oxidation of a compound. In some embodiments, the processing comprises chemical modification of a compound, e.g., glucuronidation, glycosylation.

### Purity

The “enantiomeric excess” or “% enantiomeric excess” of a composition can be calculated using the equation shown below. In the example shown below a composition contains 90% of one enantiomer, e.g., the R enantiomer, and 10% of the other enantiomer, i.e., the S enantiomer.

ee=(90−10)/100=80%.

Thus, a composition containing 90% of one enantiomer and 10% of the other enantiomer is said to have an enantiomeric excess of 80%.

In some embodiments, a provided composition contains an enantiomeric excess of at least 50%, 75%, 90%, 95%, or 99% of e.g., the R-enantiomer of the Compound (I). In other words, the composition contains an enantiomeric excess of the R enantiomer over the S enantiomer.

### Pharmaceutical Compositions

As used herein, an amount of a composition or compound effective to treat a disorder, or a “therapeutically effective amount” refers to an amount of the composition or compound which is effective, upon single or multiple dose administration to a subject, in treating a tissue, or in curing, alleviating, relieving or improving a subject with a disorder beyond that expected in the absence of such treatment.

The term “pharmaceutically acceptable carrier or adjuvant,” as used herein, refers to a carrier or adjuvant that may be administered to a subject, together with a compound of this invention, and which does not destroy the pharmacological activity thereof and is nontoxic when administered in doses sufficient to deliver a therapeutic amount of the compound.

The term, “pharmaceutically acceptable salts,” as used herein, refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. Pharmaceutically acceptable salts of the disclosure include the conventional non-toxic salts of the parent compound, e.g., Compound (I), or a pharmaceutically acceptable salt, metabolite, or prodrug thereof, formed, for example, from non-toxic inorganic or organic acids. Pharmaceutically acceptable salts of the disclosure can be synthesized from the parent compound, e.g., Compound (I), or a pharmaceutically acceptable salt, metabolite, or prodrug thereof, which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The phrase, “pharmaceutically acceptable derivative or prodrug,” as used herein refers to any pharmaceutically acceptable salt, ester, salt of an ester, or other derivative of a compound, e.g., a hydrochloride salt, which, upon administration to a recipient, is capable of providing (directly or indirectly) a therapeutic agent. For example, a prodrug may refer to a compound that is processed, in the body of a subject, into a drug. In an embodiment, the processing comprises the breaking or formation of a bond, e.g., a covalent bond. In an embodiment, the processing comprises the oxidation of a compound, e.g., hydroxylation or addition of a “—OH” group. Exemplary derivatives and prodrugs include those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. Prodrugs include derivatives where a group which enhances aqueous solubility or active transport through the gut membrane is appended to the structure of formulae described herein.

### Oral Formulations

The term, “oral dosage form,” as used herein, refers to a composition or medium used to administer an agent, e.g., Compound (I), or a pharmaceutically acceptable salt, metabolite, or prodrug thereof, to a subject. Typically, an oral dosage form is administered via the mouth, however, “oral dosage form” is intended to cover any substance which is administered to a subject and is absorbed across a membrane, e.g., a mucosal membrane, of the gastrointestinal tract, including, e.g., the mouth, esophagus, stomach, small intestine, large intestine, and colon. For example, “oral dosage form” covers a solution which is administered through a feeding tube into the stomach. “Oral dosage forms” may be administered buccally or sublingually. Oral dosage forms may comprise, in addition to Compound (I), or a pharmaceutically acceptable salt, metabolite, or prodrug thereof, a pharmaceutically acceptable carrier, one or more pharmaceutically acceptable excipients, e.g., binding agents, stabilizers, diluents, surfactants, flavors, and odorants.

The term, “dissolvable,” as used here, refers to a compound or composition whereby at least 50% (wt/wt), e.g., 70%, e.g., 80%, e.g., 90%, e.g., 98% of the compound or composition goes into solution e.g., aqueous solution, within 120 minutes when the compound or composition is placed in a preponderance of solvent, e.g., the compound or composition is placed in solvent at a ratio of at least 10:1 solvent:compound or composition (wt/wt).

Pharmaceutically acceptable carriers can be sterile liquids, e.g., water and oils, including those of petroleum, animal, vegetable or synthetic origin, such as peanut oil, soybean oil, mineral oil, sesame oil and the like. Water is a preferred carrier when the oral dosage form is a liquid. Saline solutions and aqueous dextrose and glycerol solutions can also be employed as liquid carriers. Oral dosage forms may be manufactured by processes well known in the art, e.g., by means of conventional mixing, dissolving, granulating, surface deposition, dragee-making, levigating, emulsifying, encapsulating, entrapping or lyophilizing processes. Further techniques for formulation and administration of active ingredients may be found in “Remington's Pharmaceutical Sciences,” Mack Publishing Co., Easton, Pa., latest edition, which is incorporated herein by reference as if fully set forth herein. Oral dosage forms for use in accordance with the present invention thus may be formulated in conventional manner using one or more pharmaceutically acceptable carriers comprising excipients and auxiliaries, which facilitate processing of the active ingredients into preparations which, can be used pharmaceutically.

For oral administration, the active ingredients, e.g., Compound (I), or a pharmaceutically acceptable salt, metabolite, or prodrug thereof, can be formulated readily by combining the active ingredient with pharmaceutically acceptable carriers well known in the art. Such carriers enable the active ingredients of the invention to be formulated as tablets, pills, dragees, capsules, liquids, gels, syrups, slurries, powders or granules, suspensions or solutions in water or nonaqueous media, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients such as thickeners, diluents, flavorings, dispersing aids, emulsifiers, binders or preservatives may be desirable.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1). Lower or higher doses than those recited above may be required. Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician.

Upon improvement of a subject's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Subjects may, however, require intermittent treatment on a long-term basis upon any recurrence of disease symptoms.

Oral dosage forms may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

The term, “parenteral dosage form,” as used herein, refers to a composition or medium used to administer an agent, e.g., Compound (I), or a pharmaceutically acceptable salt, metabolite, or prodrug thereof, to a subject by way other than mouth or the gastrointestinal tract. Exemplary parenteral dosage forms or modes of administration include intranasal, buccal, intravenous, intramuscular, subcutaneous, intraparenteral, mucosal, sublingual, intraoccular, and topical (e.g., intravenous or subcutaneous).

When employed as pharmaceuticals, a composition of the invention is typically administered in the form of a pharmaceutical composition. Such compositions can be prepared in a manner well known in the pharmaceutical art and comprise at least one active compound. Generally, a compound of the invention is administered in a therapeutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms.

Compositions for oral administration can take the form of bulk liquid solutions or suspensions, or bulk powders. More commonly, however, compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient, vehicle or carrier. Typical unit dosage forms include prefilled, premeasured ampules or syringes of the liquid compositions or pills, tablets, capsules or the like in the case of solid compositions. In such compositions, the active compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

The above-described components for orally administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

Compounds of the invention can also be administered in sustained release forms or from sustained release drug delivery systems. A description of representative sustained release materials can be found in Remington's Pharmaceutical Sciences.

In certain embodiments, the active agent may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

Pharmaceutical compositions can be administered with medical devices. For example, pharmaceutical compositions can be administered with a needleless hypodermic injection device, such as the devices disclosed in U.S. Pat. No. 5,399,163, 5,383,851, 5,312,335, 5,064,413, 4,941,880, 4,790,824, or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules are also known.

Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent.

In some embodiments, the pharmaceutical composition comprises a pharmaceutically acceptable excipient. In some embodiments, the pharmaceutical composition is configured in a unit dosage form. In some embodiments, the pharmaceutical composition is configured in a solid dosage form (e.g., a capsule, a tablet). In some embodiments, the solid dosage form is selected from the group consisting of tablets, capsules, sachets, powders, granules and lozenges. In some embodiments, the pharmaceutical composition is configured in a liquid dosage form. In some embodiments, the pharmaceutical composition is administered orally.

### Combinations

In some cases, provided compositions, e.g., a composition comprising the Compound (I), or a pharmaceutically acceptable salt, metabolite, or prodrug thereof, further comprise an additional agent, e.g., therapeutic agent, or are administered in combination with a composition comprising an additional agent, e.g., therapeutic agent.

In one implementation, Compound (I), or a pharmaceutically acceptable salt, metabolite, or prodrug thereof and additional agent are provided as a composition, and the composition is administered to the subject. It is further possible, e.g., at least 24 hours before or after administering the composition, to administer separately one dose of the composition comprising the Compound (I), or a pharmaceutically acceptable salt, metabolite, or prodrug thereof, and then one dose of a composition comprising an additional agent, e.g., therapeutic agent. In another implementation, the composition comprising Compound (I), or a pharmaceutically acceptable salt, metabolite, or prodrug thereof and the additional agent, e.g., therapeutic agent, are provided as separate compositions, and the step of administering includes sequentially administering the composition comprising Compound (I), or a pharmaceutically acceptable salt, metabolite, or prodrug thereof, and the composition comprising the additional agent. Sequential administrations can be provided on the same day (e.g., within one hour of one another or at least 3, 6, or 12 hours apart) or on different days.

Generally, the compositions of Compound (I), or a pharmaceutically acceptable salt, metabolite, or prodrug thereof, and the additional agent are each administered as a plurality of doses separated in time. Compositions are generally each administered according to a regimen. The regimen for one or both compositions may have a regular periodicity. The regimen for the composition comprising Compound (I), or a pharmaceutically acceptable salt, metabolite, or prodrug thereof, can have a different periodicity from the regimen for the composition comprising the additional agent, e.g., one can be administered more frequently than the other. For example, in one implementation, the composition of the Compound (I), or a pharmaceutically acceptable salt, metabolite, or prodrug thereof, and the composition of the additional agent is administered once daily and the other once weekly.

In some embodiments, each of a composition of Compound (I), or a pharmaceutically acceptable salt, metabolite, or prodrug thereof, and an additional agent is administered at the same dose as each is prescribed for monotherapy. In other embodiments, a composition of Compound (I), or a pharmaceutically acceptable salt, metabolite, or prodrug thereof, is administered at a dosage that is equal to or less than an amount required for efficacy if administered alone. Likewise, the additional agent can be administered at a dosage that is equal to or less than an amount required for efficacy if administered alone.

Non-limiting examples of additional agents for treating MD, e.g., DMD, in combination with Compound (I), or a pharmaceutically acceptable salt, metabolite, or prodrug thereof, include:

Additional agents include modulators (e.g., agonists, antagonists) of the androgen receptor. Exemplary additional agents include anabolic agents (e.g., α-methylprednisolone, nandrolone, oxandrolone), androgens (e.g., testosterone, dihydrotestosterone), myostatin-blocking agents, β2-adrenocepttor agonists, and/or selective androgen receptor modulators (SARMs). Exemplary additional agents include steroids, e.g., glucocorticosteroids, e.g., prednisone (also prednisolone), deflazacort. In some embodiments, additional agents include creatine monohydrate; glutamine; agents that bind the ribosome and cause read through of premature stop codons (nonsense mutations) such as aminoglycoside antibiotics, e.g., gentamicin; agents that cause skipping of abnormal stop codons, e.g., PTC124; or agents that force the splicing machinery of the cell to skip the dystrophin gene exon that contains the mutation, e.g., antisense RNA or morpholino antisense oligonucleotides.

Additional agents also include supplements or other drugs include co-enzyme Q10, carnitine, amino acids (e.g., glutamine, arginine), anti-inflammatories/antioxidants (e.g., fish oil, vitamin E, green tea extract, pentoxifylline), herbal or botanical extracts.

In addition to a composition of an additional agent, it is also possible to deliver other agents to the subject. However, in some embodiments, no additional agent, e.g., small molecule therapeutic, other than Compound (I), or a pharmaceutically acceptable salt, metabolite, or prodrug thereof, are administered to the subject as a pharmaceutical composition.

In some embodiments, compositions of the present invention are administered in combination with non-pharmacological management. For example, with the progression of muscle weakness, loss of respiratory muscle strength, with ensuing ineffective cough and decreased ventilation, leads to pneumonia, atelectasis, and respiratory insufficiency in sleep and while awake [Gozal 2000]. These complications are generally preventable with careful follow up and assessments of respiratory function. Patients with DMD may have routine immunizations, including the pneumococcal vaccine and annual influenza vaccine. The older ambulatory DMD boys may have annual spirometry measures. Once the child is wheelchair bound and if his force vital capacity (FVC) falls below 80% predicted, and/or the child is 12 years of age, he may be seen twice a year by a physician specializing in pediatric respiratory care [Finder et al. 2004]. More advanced patients who require mechanically assisted airway clearance therapy or mechanically assisted ventilation may see a pulmonologist every 3 to 6 months. Routine evaluations at these visits may include oxyhemoglobin saturation by pulse oxymetry, spirometry, and measures of inspiratory and expiratory pressures and peak cough flow [Bach et al. 1997]. The use of assisted cough technologies may be recommended when peak cough flow is less than 270 L/minute and/or whose maximal expiratory pressures are less than 60 cm H₂O [Finder et al. 2004]. DMD patients have increase risk for sleep apnea, nocturnal hypopneas and hypoxemia. Treatment of these with noninvasive nocturnal ventilation can significantly increase quality of life [Baydur et al. 2000].

While high-resistance exercise, especially those involving eccentric contractions (i.e. weight lifting) may be damaging to the muscle cell membrane and should be avoided [Ansved 2003], a sedentary life may be equally damaging [McDonald 2002]. Keeping an active lifestyle, e.g., non-resistive exercises such as swimming, may prevent excessive weight gain, especially if the child is on steroids. Swivel walkers may be used to provide low-energy ambulation and improve life quality.

Contractures of the Achilles tendons, and later of other joints, are common. Active range of motion exercises supplemented by passive stretching is important to prevent contractures early on and maintain better gait mechanics. A standing board may be used for non-ambulant boys to provide constant stretching of the Achilles tendons. If strenuous stretching is not effective, surgical release of tight heel cords may be beneficial [Bushby 2010b]. Long leg bracing can be offered to keep some ambulation after contractures are corrected. The iliotibial bands may also tighten because of broad-based gait used to maintain stability. The hip flexors may become contracted when ambulation is still present as a result of the anterior rotation of the hips or later because of sitting for prolonged periods in a wheelchair. Hip flexion contractures may benefit from surgical release followed by application of long leg braces. Resection of the fascia lata (Rideau procedure) may be beneficial for some patients [Do 2002].

Many patients with DMD develop scoliosis after losing independent ambulation. The use of solid seat and back inserts in properly fitted wheelchairs may be helpful in preventing scoliosis by keeping truncal posture erect. For some boys, long leg braces can be fitted to allow braced upright daily standing to prevent curvature. The use of steroids, perhaps because it prolongs ambulation beyond the growth spur of early teenage years, delays or prevents scoliosis, even if the child is eventually wheelchair bound [Alman et al. 2004; Yilmaz et al. 2004]. Once scoliosis reaches 30 degrees, it typically progresses with age and growth. Failure to repair scoliosis in DMD can result in increased hospitalization rates, worsening or pulmonary function and poor quality of life [bFinder et al. 2004]. Surgical intervention may occur while lung and cardiac function are satisfactory (with the best recovery generally when FVC is >40%), however there are no absolute contraindications for scoliosis surgery based on pulmonary function [Finder et al. 2004]. Surgery is usually scheduled once the Cobb angle measured on scoliosis films is between 30 and 50 degrees [Brook et al. 1996].

A correlation of cardiac involvement with prognosis of DMD may be made by measuring left ventricular dysfunction by echocardiography [Corrado et al. 2002]. Recent guidelines for the study of cardiac involvement in DMD [Bushby 2003; Finsterer and Stollberger 2003; Bushby 2010b] recommend an EKG and echocardiography at the time of diagnosis and then screened every 2 years up to age 10 and subsequently every year. The early, preventive use of ACE inhibitors and later beta-blockers may be used if needed [Bushby 2003; Finsterer and Stollberger 2003].

### Administration and Dosage

### Methods of Administration

Inventive methods of the present invention contemplate single as well as multiple administrations of a therapeutically effective amount of a composition as described herein. Compositions, e.g., a composition as described herein, can be administered at regular intervals, depending on the nature, severity and extent of the subject's condition. In some embodiments, a composition described herein is administered in a single dose. In some embodiments, a composition described herein is administered in multiple doses. In some embodiments, a therapeutically effective amount of a composition, e.g., a composition described herein, may be administered orally and periodically at regular intervals (e.g., 1, 2, 3, 4, 5, 6, 7, 8, 9, 10 or more times every 1, 2, 3, 4, 5, or 6 days, or every 1, 2, 3, 4, 5, 6, 7, 8, or 9 weeks, or every 1, 2, 3, 4, 5, 6, 7, 8, 9 months or longer).

In some embodiments, a compositions described herein is administered at a predetermined interval (e.g., 1, 2, 3, 4, 5, 6, 7, 8, 9, 10 or more times every 1, 2, 3, 4, 5, or 6 days, or every 1, 2, 3, 4, 5, 6, 7, 8, or 9 weeks, or every 1, 2, 3, 4, 5, 6, 7, 8, 9 months or longer). In some embodiments, a composition is administered chronically.

In some embodiments, a composition is administered once daily.

Dosage levels of from about 0.01 to about 100 mg/kg body weight per day, preferably from about 0.01 to about 10 mg/kg body weight per day are useful for the treatment of MD, e.g., DMD. In some embodiments, dosage levels are from about 0.01 to about 5 g/day, for example from about 0.025 to about 2 g/day, from about 0.05 to about 1 g/day, per subject (based on the average size of a subject calculated at about 20 kg). Typically, the pharmaceutical compositions of, and according to, this invention will be administered from about 1 to about 5 times per day, preferably from about 1 to about 3 times per day.

In some embodiments, Compound (I) or pharmaceutically acceptable salt, metabolite, or prodrug thereof is administered chronically. In some embodiments, Compound (I) or pharmaceutically acceptable salt, metabolite, or prodrug thereof is administered 1, 2, 3, 4, 5, 6, 7, 8, 9, 10 times or more every 1, 2, 3, 4, 5, 6, days, 1, 2, 3, 4, 5, 6, 7, 8, 9 weeks, 1, 2, 3, 4, 5, 6, 7, 8, 9 months or more. In some embodiments, Compound (I) or pharmaceutically acceptable salt, metabolite, or prodrug thereof is administered once daily.

In some embodiments, the dose of Compound (I), or a pharmaceutically acceptable salt, metabolite, or prodrug thereof can be a dose, e.g., about 0.1 mg to about 10 mg a day, e.g., about 0.25 mg or about 1 mg a day. For example, a dose of about 0.5/day of Compound (I), or a pharmaceutically acceptable salt, metabolite, or prodrug thereof can be administered to a patient, e.g., as a 0.5 mg dose once a day. In some embodiments, the 0.5 mg dose is in an about 5 mg, 10 mg, 20 mg, 25 mg, 30 mg, 50 mg, 75 mg, 100 mg, 150 mg, 200 mg or larger tablet. As an example, a dose of about 0.5 mg/day of Compound (I), or a pharmaceutically acceptable salt, metabolite, or prodrug thereof can be administered to a patient, e.g., about 0.25 mg administered two times a day.

In some embodiments, Compound (I), or a pharmaceutically acceptable salt, metabolite, or prodrug thereof is administered in a dose of about 0.1 mg to 1 mg per subject, about 0.2 mg to about 0.8 mg per subject, about 0.3 mg to about 0.7 mg per subject, about 0.4 mg to about 0.6 mg per subject.

In some embodiments, Compound (I), or a pharmaceutically acceptable salt, metabolite, or prodrug thereof is administered at a dose of no more than 1 mg, 0.5 mg, 0.25 mg, or 0.1 mg per subject. In some embodiments, the dose is 0.1 mg per subject. In some embodiments, the dose is 0.25 mg per subject. In some embodiments, the dose is 0.5 mg per subject. In some embodiments, the dose is 1 mg per subject.

In some embodiments, Compound (I), or a pharmaceutically acceptable salt, metabolite, or prodrug thereof is administered in a dose of about 0.1 ng to about 1 g per kg subject weight, about 100 ng to about 10 mg per kg subject weight, about 1 μg to about 100 μg per kg subject weight, about 5 μg to about 25 μg, about 10 μg to about 20 μg, or about 3 μg to about 30 μg per kg subject weight. In some embodiments, Compound (I), or a pharmaceutically acceptable salt, metabolite, or prodrug thereof is administered at a dose of no more than 250 μg, 150 μg, 100 μg, 50 μg, 30 μg, 15 μg, 7 μg, or 3 μg per kilogram subject weight.

In some embodiments, Compound (I), or a pharmaceutically acceptable salt, metabolite, or prodrug thereof is administered at a dose of about 3 μg per kilogram subject weight. In some embodiments, Compound (I), or a pharmaceutically acceptable salt, metabolite, or prodrug thereof is administered at a dose of about 7 μg per kilogram subject weight. In some embodiments, Compound (I), or a pharmaceutically acceptable salt, metabolite, or prodrug thereof is administered at a dose of about 15 μg per kilogram subject weight (e.g.,). In some embodiments, Compound (I), or a pharmaceutically acceptable salt, metabolite, or prodrug thereof is administered at a dose of about 30 μg per kilogram subject weight.

In some embodiments, Compound (I), or a pharmaceutically acceptable salt, metabolite, or prodrug thereof is administered in a single dose.

In some embodiments, the pharmaceutical composition described herein is provided in an oral dosage form, e.g., an oral dosage form as described herein. In some embodiments, the oral dosage form contains at least about 10%, at least about 20%, at least about 30%, at least about 40%, at least about 50%, at least about 60%, at least about 70%, at least about 80%, at least about 90% or greater of Compound (I), or a pharmaceutically acceptable salt, metabolite, or prodrug thereof.

In some embodiments, Compound (I), or a pharmaceutically acceptable salt, metabolite, or prodrug thereof is not 100% potent or pure (e.g., the potency or purity is at least about 75%, at least about 80%, at least about 90%, at least about 92%, at least about 95%, at least about 98%, at least about 99% potent), in which case the doses described above refer to the amount of potent or pure Compound (I), or a pharmaceutically acceptable salt, metabolite, or prodrug thereof administered to a patient rather than the total amount of Compound (I), or a pharmaceutically acceptable salt, metabolite, or prodrug thereof. These doses can be administered to a patient as a monotherapy and/or as part of a combination therapy, e.g., as described herein.

Such administration can be used as a chronic therapy. The amount of active ingredient that may be combined with the carrier materials to produce a single dosage form and may vary depending upon the subject treated. A typical preparation will contain from about 5% to about 95% active Compound (w/w). Preferably, such preparations contain from about 20% to about 80%, from about 25% to about 70%, from about 30% to about 60% active Compound (w/w).

When the compositions of this disclosure involve a combination of the Compound (I), or a pharmaceutically acceptable salt, metabolite, or prodrug thereof and one or more additional therapeutic or prophylactic agents, both the compound and the additional agent should be present at dosage levels of between about 10 to 100%, and more preferably between about 10 to 80% of the dosage normally administered in a monotherapy regimen.

Upon improvement of a patient's condition, a maintenance dose of a composition as described herein may be administered, if necessary. Subsequently, the dosage or frequency of administration, or both, may be reduced, e.g., to about ½ or ¼ or less of the dosage or frequency of administration, as a function of the symptoms, to a level at which the improved condition is retained when the symptoms have been alleviated to the desired level, treatment should cease. Patients may, however, require intermittent treatment on a long-term basis upon any recurrence of disease symptoms.

It should also be understood that a specific dosage and treatment regimen of any particular patient will depend upon a variety of factors, including the activity of the specific compound employed, the age, body weight, general health, diet, time of administration, rate of excretion, drug combination, and the judgment of the treating physician and the severity of the disease treated. The amount of active ingredients will also depend upon the particular described compound and the presence or absence and the nature of the additional agent in the composition.

### Food Effect

Provided compositions and methods may be affected by meal consumption, by the treated subject. For example, meal consumption may lead to an increase or decrease in the effectiveness or therapeutic activity of the treatment. For example, meal consumption can affect therapeutic activity by e.g., increasing or decreasing the bioavailability of a compound, e.g., compound as described herein; affect the ability of a compound, e.g., compound as described herein, to modulate a protein, e.g., receptor (e.g., AR). It will be appreciated that the term “meal consumption” generally refers to intake of nutrients, for example, by intake of calorie-containing liquids or solids. In some embodiments, a meal can be a glass of milk or other protein-containing drink. In general, compositions to be administered with a meal are not to be taken during a fasting period, e.g., a period of fasting of 2, 3, 4, 5, 6, 7, 8, 9, 10, 11, 12 or more hours. Compositions to be administered in the absence of a meal are to be taken during a fasting period, e.g., a period of fasting of 2, 3, 4, 5, 6, 7, 8, 9, 10, 11, 12 or more hours.

In some embodiments, the compositions described herein are administered after meal consumption. In some embodiments, the compositions described herein are administered at least 10 minutes, at least 20 minutes, at least 30 minutes, at least 45 minutes, at least 60 minutes, at least 90 minutes, at least 120 minutes, at least 3 hours, at least 4 hours, at least 6 hours, after meal consumption.

In some embodiments, the compositions described herein are administered before meal consumption. In some embodiments, the compositions described herein are administered at least 10 minutes, at least 20 minutes, at least 30 minutes, at least 45 minutes, at least 60 minutes, at least 90 minutes, at least 120 minutes, at least 3 hours, at least 4 hours, at least 6 hours, before meal consumption.

### Kits

In another aspect, the invention features kits for evaluating a sample, e.g., a sample from an MD, e.g., DMD patient, to detect or determine the level of one or more genes as described herein. The kit includes a means for detection of (e.g., a reagent that specifically detects) one or more genes as described herein. In certain embodiments, the kit includes an MD, e.g., DMD therapy.

The methods, devices, reaction mixtures, kits, and other inventions described herein can further include providing or generating, and/or transmitting information, e.g., a report, containing data of the evaluation or treatment determined by the methods, assays, and/or kits as described herein. The information can be transmitted to a report-receiving party or entity (e.g., a patient, a health care provider, a diagnostic provider, and/or a regulatory agency, e.g., the FDA), or otherwise submitting information about the methods, assays and kits disclosed herein to another party. The method can relate to compliance with a regulatory requirement, e.g., a pre- or post approval requirement of a regulatory agency, e.g., the FDA. In one embodiment, the report-receiving party or entity can determine if a predetermined requirement or reference value is met by the data, and, optionally, a response from the report-receiving entity or party is received, e.g., by a physician, patient, diagnostic provider.

A compound of the invention described herein may be provided in a kit. The kit includes a composition provided herein, e.g., composition comprising Compound (I) or a pharmaceutically acceptable salt, prodrug, or metabolite thereof described herein and, optionally, a container, a pharmaceutically acceptable carrier and/or informational material. The informational material can be descriptive, instructional, marketing or other material that relates to the methods described herein and/or the use of the α4 antagonist for the methods described herein.

The informational material of the kits is not limited in its form. In one embodiment, the informational material can include information about production of the composition provided herein, e.g., composition comprising Compound (I), or a pharmaceutically acceptable salt, prodrug, or metabolite thereof, physical properties of the compound, concentration, date of expiration, batch or production site information, and so forth. In one embodiment, the informational material relates to methods for administering the composition provided herein, e.g., composition comprising Compound (I), or a pharmaceutically acceptable salt, prodrug, or metabolite thereof, e.g., by a route of administration described herein and/or at a dose and/or dosing schedule described herein.

In one embodiment, the informational material can include instructions to administer a composition provided herein, e.g., composition comprising Compound (I), or a pharmaceutically acceptable salt, prodrug, or metabolite thereof described herein in a suitable manner to perform the methods described herein, e.g., in a suitable dose, dosage form, or mode of administration (e.g., a dose, dosage form, or mode of administration described herein). In another embodiment, the informational material can include instructions to administer a composition provided herein, e.g., composition comprising Compound (I), or a pharmaceutically acceptable salt, prodrug, or metabolite thereof to a suitable subject, e.g., a human, e.g., a human having a muscular dystrophy, e.g., a human having DMD.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a composition provided herein, e.g., composition comprising Compound (I), or a pharmaceutically acceptable salt, prodrug, or metabolite thereof as described herein and/or its use in the methods described herein. The informational material can also be provided in any combination of formats.

In addition to a composition provided herein, e.g., composition comprising Compound (I), or a pharmaceutically acceptable salt, prodrug, or metabolite thereof as described herein, the composition of the kit can include other ingredients, such as a surfactant, a lyoprotectant or stabilizer, an antioxidant, an antibacterial agent, a bulking agent, a chelating agent, an inert gas, a tonicity agent and/or a viscosity agent, a solvent or buffer, a stabilizer, a preservative, a pharmaceutically acceptable carrier and/or a second agent for treating a condition or disorder described herein. Alternatively, the other ingredients can be included in the kit, but in different compositions or containers than a composition provided herein, e.g., composition comprising Compound (I), or a pharmaceutically acceptable salt, prodrug, or metabolite thereof as described herein.

In some embodiments, a component of the kit is stored in a sealed vial, e.g., with a rubber or silicone closure (e.g., a polybutadiene or polyisoprene closure). In some embodiments, a component of the kit is stored under inert conditions (e.g., under Nitrogen or another inert gas such as Argon). In some embodiments, a component of the kit is stored under anhydrous conditions (e.g., with a desiccant). In some embodiments, a component of the kit is stored in a light blocking container such as an amber vial.

A composition provided herein, e.g., composition comprising Compound (I), or a pharmaceutically acceptable salt, prodrug, or metabolite thereof described herein can be provided in any form, e.g., liquid, frozen, dried or lyophilized form. It is preferred that a composition including the composition provided herein, e.g., composition comprising a SARM, e.g., Compound (I), or a pharmaceutically acceptable salt, prodrug, or metabolite thereof described herein be substantially pure and/or sterile. When a composition provided herein, e.g., composition comprising Compound (I), or a pharmaceutically acceptable salt, prodrug, or metabolite thereof described herein is provided in a liquid solution, the liquid solution preferably is an aqueous solution, with a sterile aqueous solution being preferred. In one embodiment, the composition provided herein, e.g., composition comprising a SARM, e.g., Compound (I), or a pharmaceutically acceptable salt, prodrug, or metabolite thereof is supplied with a diluents or instructions for dilution. The diluent can include for example, a salt or saline solution, e.g., a sodium chloride solution having a pH between 6 and 9, lactated Ringer's injection solution, D5W, or PLASMA-LYTE A Injection pH 7.4® (Baxter, Deerfield, Ill.).

The kit can include one or more containers for the composition containing a composition provided herein, e.g., composition comprising Compound (I), or a pharmaceutically acceptable salt, prodrug, or metabolite thereof described herein. In some embodiments, the kit contains separate containers, dividers or compartments for the composition and informational material. For example, the composition can be contained in a bottle, vial, IV admixture bag, IV infusion set, piggyback set or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. The containers of the kits can be air tight, waterproof (e.g., impermeable to changes in moisture or evaporation), and/or light-tight.

The invention is further illustrated by the following examples, which should not be construed as further limiting.

### EXAMPLES

### Example 1

### Multidisciplinary Assessment of the Effect of In Vivo Treatment of Compound (I) in Comparison with Nandrolone and α-Methy-Prednisolone (PDN) on the Model of Exercised Mdx Mice

### Introduction

The present study is aimed at testing, by means of multidisciplinary in vivo and ex vivo approaches, the effects of Compound (I), a selective androgen receptor modulator (SARM) with muscle specific action, on the model of chronically exercised mdx mice. In agreement with the clinical use of glucocorticoids in Duchenne patients, the effects of Compound (I) (30 mk/kg, s.c. 6 day/week) were compared with those of a parallel treatment with α-methyl-prednisolone (PDN) (1 mg/kg i.p. 6 days/week) as well as with those of the anabolic drug nandrolone (5 mg/kg, s.c. 6 day/week).

The experiment describes the results obtained by ex vivo determination of primary functional and morphological end-points, revising them in relation to the methodological approach and in vivo data.

### Methods

Compound (I) and nandrolone were dissolved in 10% Ethanol/90% Corn Oil (Sigma-Aldrich) in order to have the final dosage needed in the volume of 0.1 ml/10 g body weight. PDN (from the commercial formulation URBASON) was diluted sterile water for injection (0.1 ml/10 g body weight). 40 mdx mice 5-6 weeks old (Charles River Italy for Jackson Lab) were first (at the beginning of the exercise/treatment period (Time 0)) randomized in groups homogeneous for body weight, fore limb strength and normalized force (fore limb strength/body weight), as follows:
7 SEDENTARY MDX+VEHICLE (ethanol and corn oil)
7 UNTREATED EXERCISED MDX+VEHICLE (ethanol and corn oil)
9 EXERCISED MDX+Compound (I) 30 mg/kg
8 EXERCISED MDX+Nandrolone 5 mg/kg
3 EXERCISED MDX+VEHICLE (sterile water)
6 EXERCISED MDX+PDN 1 mg/kg

The duration of the treatment was 4-6 weeks. At the end of 4 weeks, 34 mice remained on the protocol, as follows:
7 SEDENTARY MDX+VEHICLE (ethanol and corn oil)
7 UNTREATED EXERCISED MDX+VEHICLE (ethanol and corn oil)
6 EXERCISED MDX+Compound (I) 30 mg/kg
5 EXERCISED MDX+Nandrolone 5 mg/kg
3 EXERCISED MDX+VEHICLE (sterile water)
6 EXERCISED MDX+PDN 1 mg/kg

The chronic exercise consisted of 30 min running on horizontal treadmill at 12 m/min twice a week. Drug treatment started one day before the exercise protocol. At least 4 weeks of exercise were performed before starting the ex vivo experiments. In vivo parameters were monitored weekly throughout. Age of the mice at the time of ex-vivo experiments: 9-12 weeks. The effectiveness of the test compounds was then evaluated ex vivo for:
Mechanical properties of EDL muscle and diaphragm strips by isometric contraction
Mechanical threshold of EDL muscle, i.e. the voltage threshold for fiber contraction, as an index of excitation-contraction coupling mechanism and calcium homeostasis (two microelectode “point” voltage clamp method)
Cable parameters and macroscopic ionic conductance (two microelectrode current clamp recordings)
Spectrophotometric determination of plasma level of creatine kinase (CK), as an index of sarcolemmal damage, lactate dehydrogenase (LDH) as an index of metabolic sufferance, reactive oxygen species, as a marker of oxidative stress
Morphometric analysis of gastrocnemious (GC) muscle and diaphragm

Values are expressed as mean±S.E.M. Statistical analysis was made by ANOVA test of variance for multiple comparison, followed by post-hoc Bonferroni's t test. Student's t test was also used for comparison between two groups.

Plasma samples were stored and later analyzed for plasma level of Compound (I). Whole-leg bones were dissected, cleaned of surrounding tissue and frozen at −80° C. Controlateral tibiae were also collected, cleaned and stored in ethanol 40% at 4° C. Bone samples used for bone density and morphology analyses. Organs that are either possible targets of SARM action (heart, prostate, levator anii, soleus) or possible markers of toxic drug action (liver, kidney, spleen) were also collected and weighed. Extra muscle samples (GC, TA, Diaphragm strips) were collected, snap frozen in liquid nitrogen or frozen in cooled isopentane and stored at −80° C. for further eventual biochemical analyses (pro-fibrotic and/or pro-inflammatory cytokines and/or growth and transcription factors by ELISA) or for immunohistochemistry (DHE staining, NF-kB staining, utrophin).

### Results

### Mechanical Properties of EDL and Diaphragm Muscle by Isometric Contraction

Standard isometric contraction measurements on both EDL muscle and diaphragm strips were obtained by electric field stimulation via two axial platinum wires. For each preparation, a preliminary stabilization procedure (for proper temperature equilibration and relaxation after handling) was followed by the determination of the optimal resting length, i.e. the resting tension that allowed the maximal tension to be elicited by 40V depolarizing steps of 0.2 ms duration. The preparations were then allowed to rest for about 30 min before starting the recording procedure.

### Diaphragm

Twitch tension: 5 single twitches elicited by pulses of 40 V and 0.2 ms (every 30 sec)→determination of maximal twitch tension and contraction kinetic (time to peak and half relaxation time);

Force frequency curve: 450 ms trains of 0.2 ms 40V pulses from 10 to 140 Hz→determination of maximal tetanic tension and frequency for half-maximal activation (Hz50) Fatigue: 5 tetani at 100 Hz (450 ms) and 5 sec intervals→determination of % drop of tension

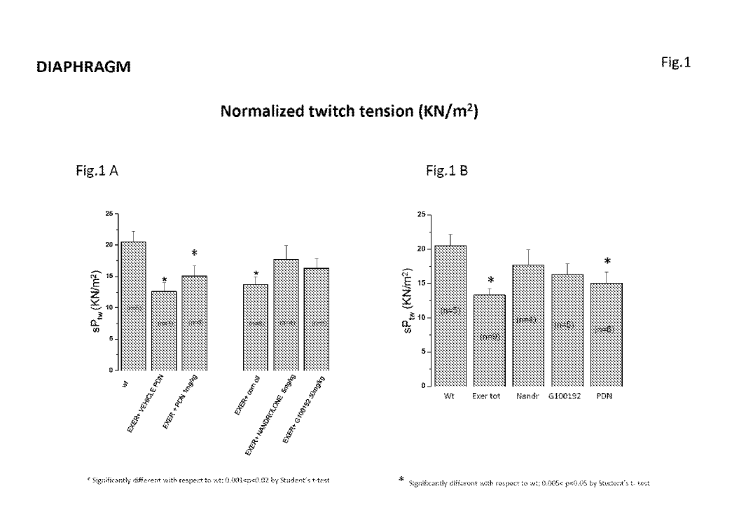

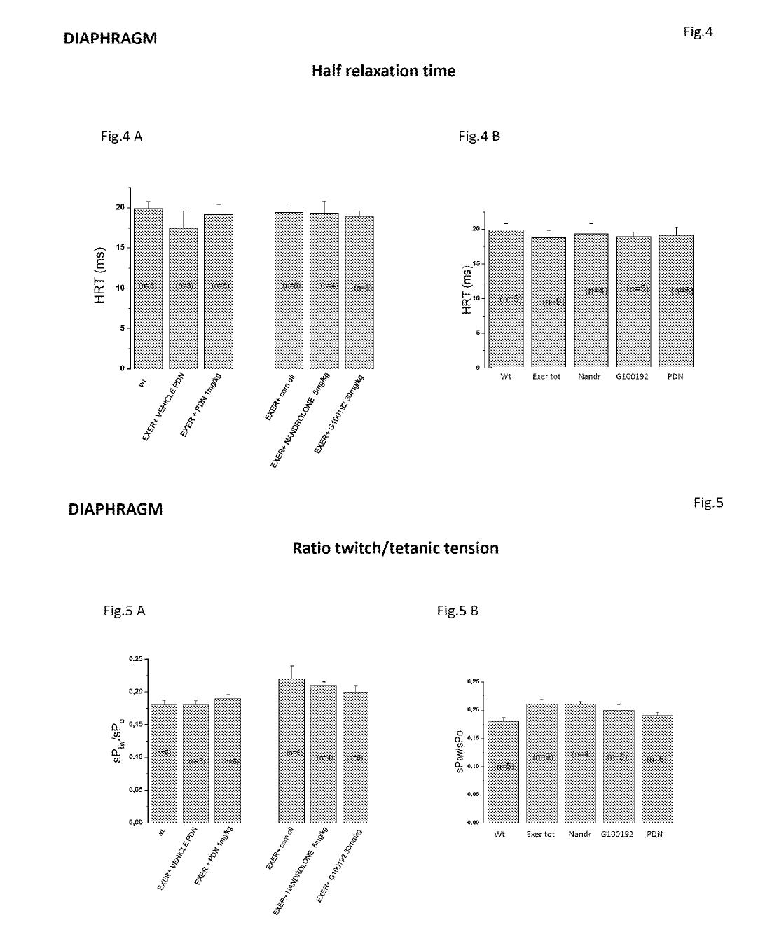

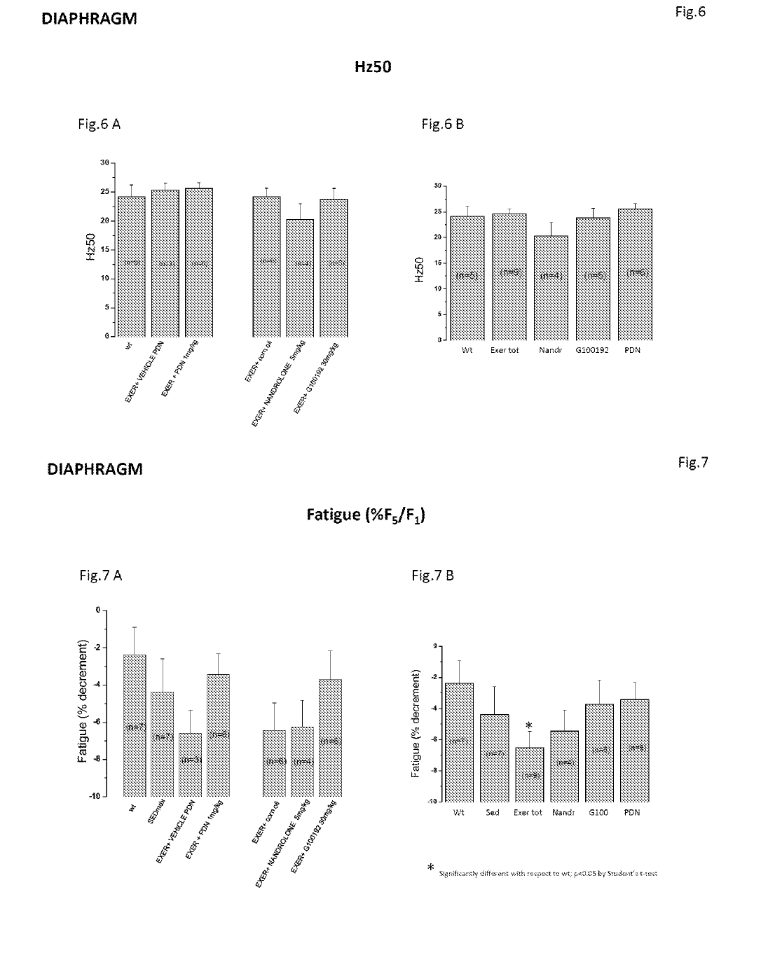

The effects of the drug treatments on contractile properties of diaphragm are shown in FIGS. 1 to 7. For each Figure the panels B show the values from the two vehicle-treated groups of exercised mdx mice pooled together. Mean and individual values of absolute and normalized twitch and tetanic tension and contractile kinetics are also provided.

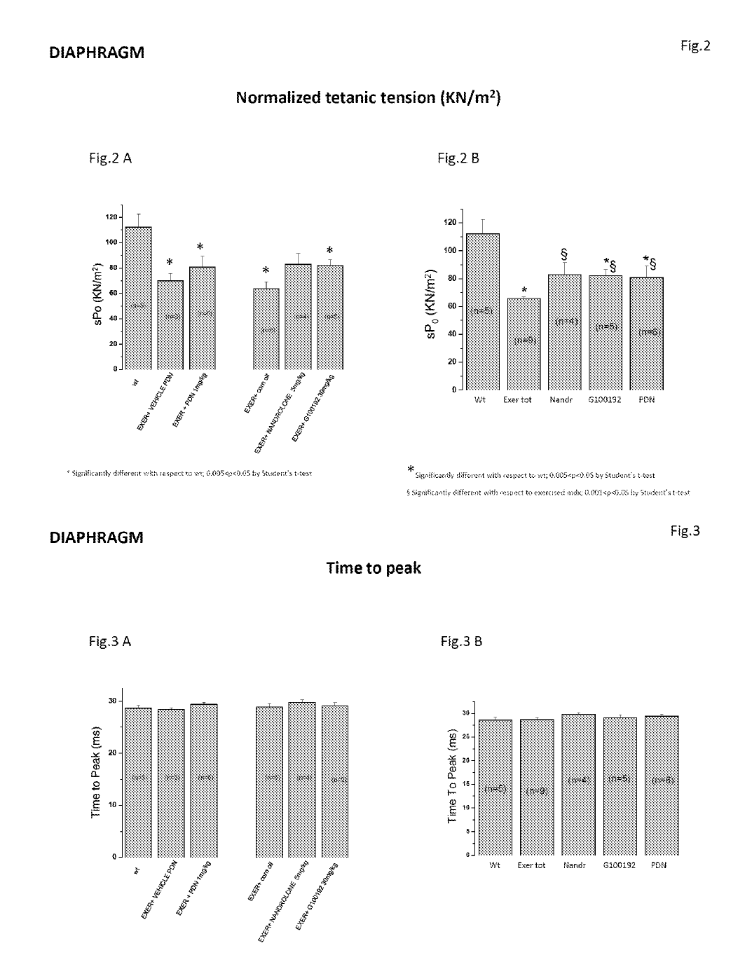

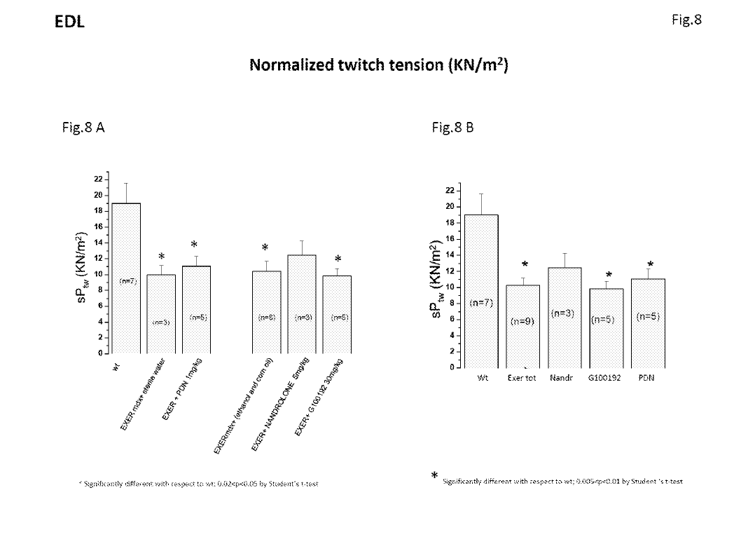

Both twitch and tetanic tension were significantly lower in diaphragm strips of exercised mdx mice with respect to weight (FIGS. 1 and 2). The twitch tension values of Compound (I) and nandrolone-treated diaphragm strips were greater than those of untreated ones and no more significantly different with respect to those of weight. On this parameter the two anabolic compounds exerted a greater protection than PDN. A clear trend toward an increase in tetanic force was also observed in the drug-treated groups, and especially in the mice treated with the two anabolic compounds. When the groups of vehicle-treated exercised mdx mice were pooled together, the values of tetanic force of Compound (I), nandrolone and PDN treated diaphragms were significantly greater than those of untreated, although still lower than weight ones (FIG. 2B). No significant differences were observed in calcium-dependent parameters (twitch/tetanic ratios and Hz50) nor in the contractile kinetics (time-to peak, relaxation time, etc) between experimental groups (FIGS. 3-6). Diaphragm muscles of mdx mice were more fatigable than weight ones and a further increase of fatigue was observed in the exercised group (FIG. 7). Interestingly, protection was observed in drug-treated groups, specifically with Compound (I) and PDN treatment. In these two groups the drop of force after 5 repetitive tetani was not significantly different with respect to that of weight.

### EDL Muscle

Twitch tension: 5 single twitches elicited by pulses of 40 V and 0.2 ms (every 30 sec)→determination of maximal twitch tension and contraction kinetic (time to peak and half relaxation time);

Force frequency curve: 350 ms trains of 0.2 ms 40V pulses from 10 to 140 Hz→determination of maximal tetanic tension and frequency for half-maximal activation (Hz50) Fatigue: 5 tetani at 100 Hz (350 ms) and 5 sec intervals→determination of % drop of tension

The results are shown in FIGS. 8-14. For each Figure the panels B show the values from the two vehicle-treated groups of exercised mdx mice pooled together. Mean and individual values of absolute and normalized twitch and tetanic tension and contractile kinetics are also provided.

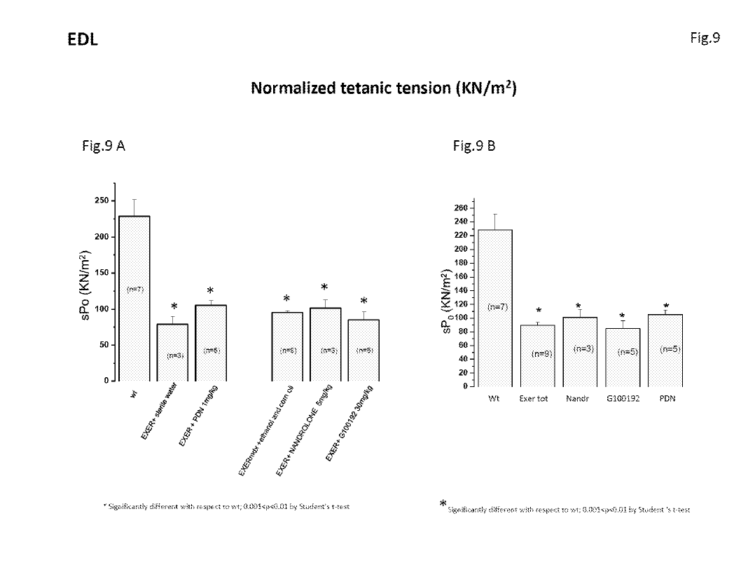

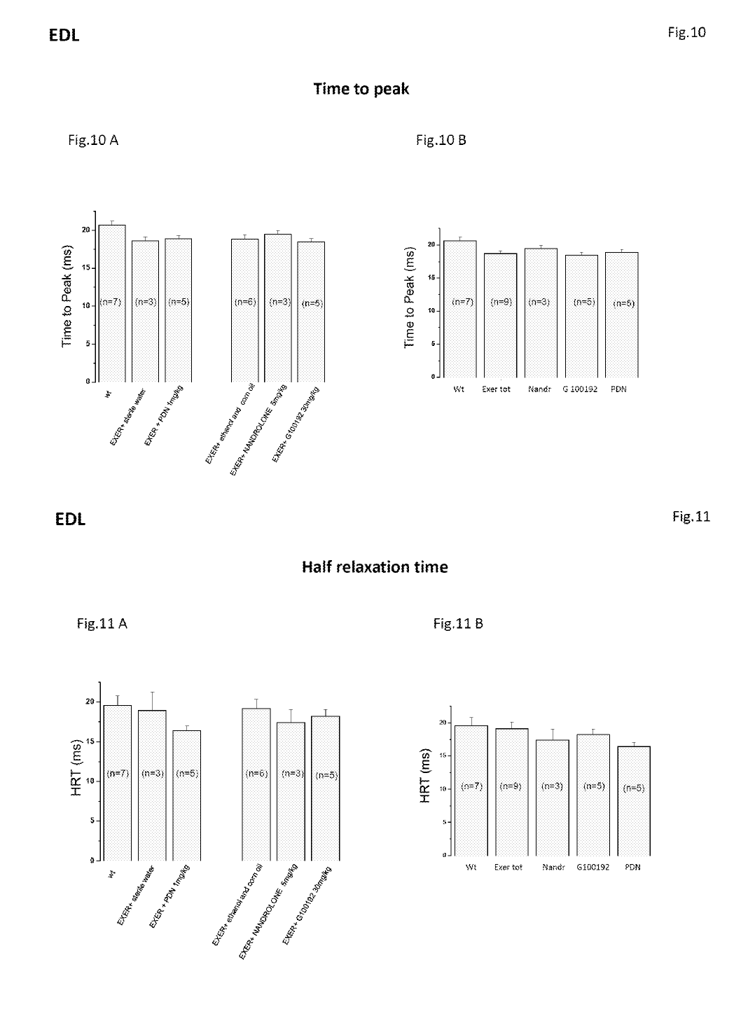

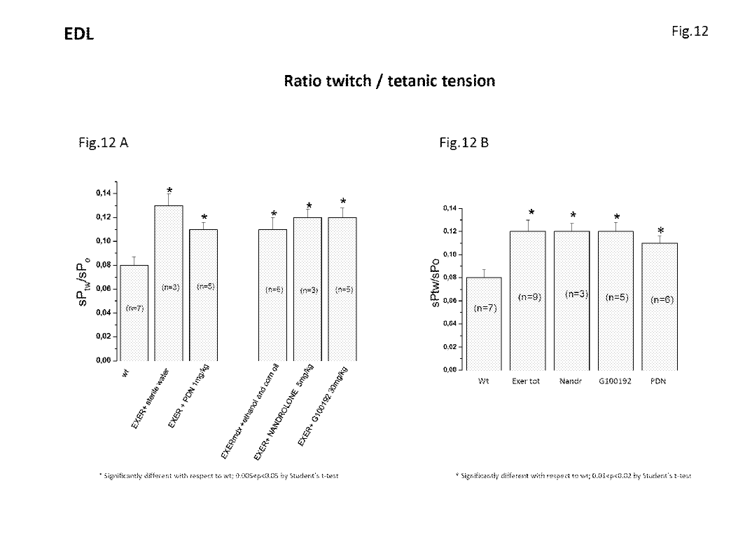

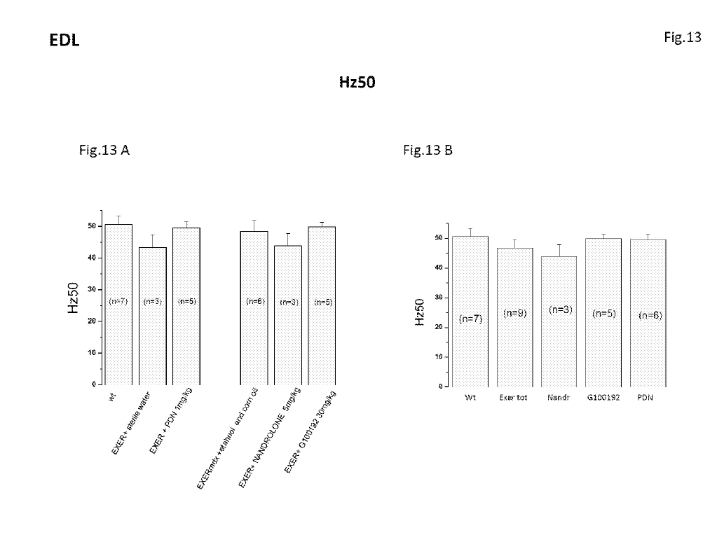

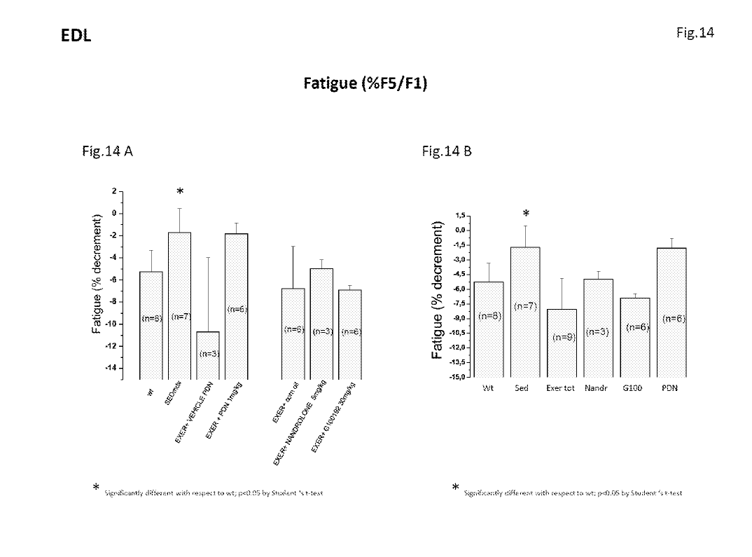

Mdx EDL muscles, either sedentary or exercised, showed significantly lower values of normalized twitch and tetanic tension with respect to weight EDL muscles. A slight decrease of muscle force was observed in exercised vs. sedentary animals. No significant amelioration was observed on twitch or tetanic forces, as both absolute and normalized values, in the groups of drug-treated animals (FIGS. 8-9). A tendency toward an increase in twitch tension was observed in nandrolone-treated group (FIG. 8). No significant differences were observed between experimental groups for contraction and relaxation times (FIGS. 10-11). The parameters that are indices of calcium homeostasis, and in particular the twitch/tetanus ratio and the force-frequency curve were then determined. The twitch/tetanus ratio is significantly increased in untreated exercised mdx vs. weight EDL muscle; this is in line with the described increase in cytosolic calcium level. However, no effect was observed in either Compound (I) nor in nandrolone treated animals, with a slight but not significant decrease being observed in PDN treated group (FIG. 12). Similarly, no significant effects were observed on the frequency for half-maximal activation (FIG. 13).

The possible ability of the treatment to protect the muscle against the fatigue was then tested. 250 ms tetani were applied at 5 sec intervals, focusing on the drop occurring during the first 5 tetani, since this represents the dynamic phase of fatigue. The exercised mdx EDL muscles fatigue more than weight and mdx sedentary ones, these latter showing an unexpected resistance to fatigue, which maybe related to the active regeneration occurring in limb muscles around this age (De Luca et al. 2003). Interestingly, a partial recovery of this parameter was observed with nandrolone and also with PDN. In fact, the muscles treated with PDN showed values similar to those of sedentary mdx (FIG. 14B). No significant protection by any of the treatment was found on the eccentric contraction, as a similar 60-70% drop of force was observed after 10 stretched protocols (20% stretch over resting tension during maximal tetanic contraction) in all groups (data not shown).

### Electrophysiological Recordings on EDL Muscle

Mechanical threshold (MT) is an electrophysiological index of excitation contraction-coupling and of calcium homeostasis (De Luca et al., JPET 2003; Fraysse et al., Neurobiol. Dis., 2004). The application of depolarizing voltage steps of increasing durations shifts the contraction for fiber contraction toward more negative potential until a constant rheobase voltage is reached. The rheobase voltage represents the voltage at which the calcium that is released from the sarcoplasmis reticulum and the calcium reuptaken are at the steady-state. A shift of rheobase voltage toward more negative potential, as occurring in dystrophic mdx EDL myofibers, is indicative of more calcium being available for contraction, either resulting from greater release or slower reuptake or higher basal cytosolic levels.

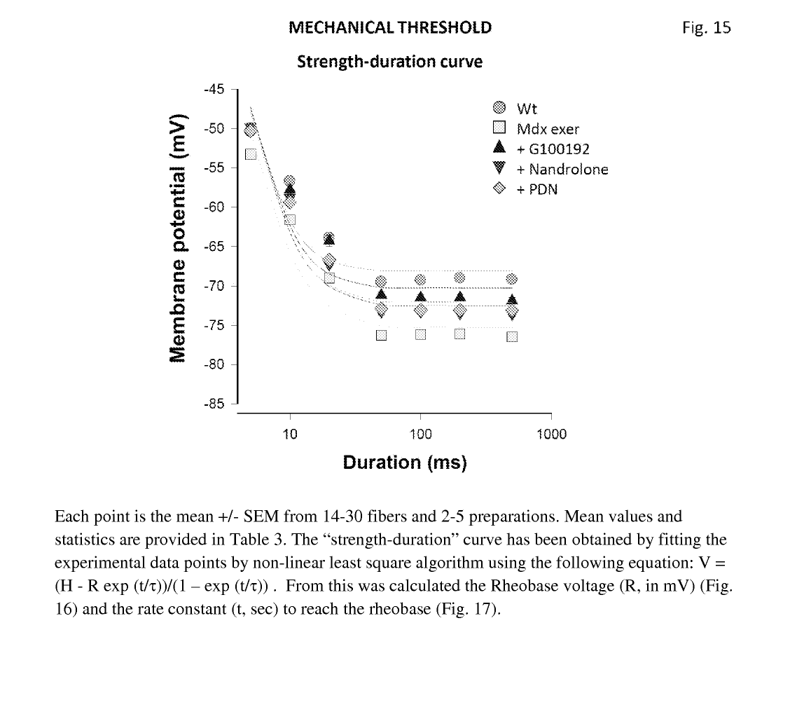

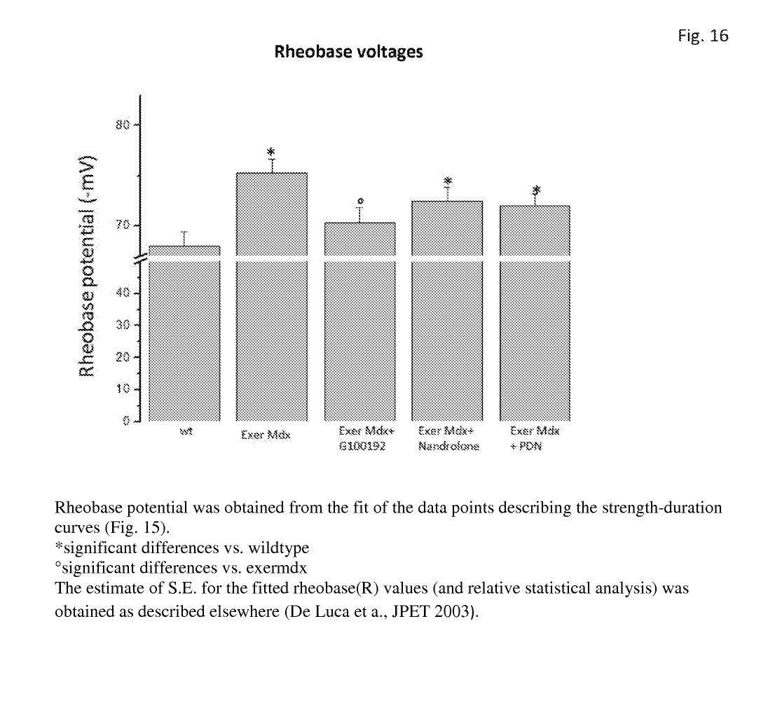

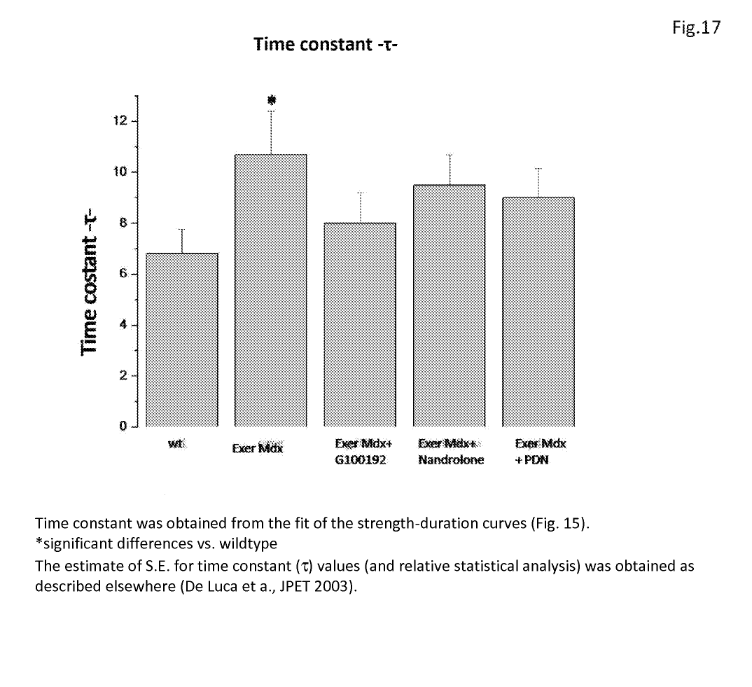

The effects of the drug treatments on MT are shown in FIGS. 15-17. As can be seen, the treatment with Compound (I) led to a significant shift of the potential for fiber contraction toward weight values at all durations of the depolarizing pulses. Both nandrolone and PDN were less effective than Compound (I) (FIG. 15). In fact the rheobase voltage calculated from the fit of the data points showed that the rheobase voltage of Compound (I) treated EDL muscles was almost overlapping that of weight, while nandrolone and PDN showed values intermediate between untreated mdx and weight (FIG. 16). An amelioration was also observed in the kinetic process for reaching the equilibrium. In fact the time constant to reach the rheobase for the Compound (I)-treated EDL myofibers was remarkably shorter than those of untreated exercised and not significantly different with respect to that of weight (FIG. 17). The effect of Compound (I) on this parameter was greater than that of nandrolone and PDN. The effect of PDN on both strength-duration curve, rheobase voltage and time constant was consistent with that which was observed in previous trials (De Luca et al., JPET, 2003).

### Cable Parameters

Passive cable properties are calculated for the spatial and temporal changes of membrane potential in response to a hyperpolarizing square current pulses. These changes are dependent of fiber diameter, membrane capacitance and membrane resistance that can be calculated from the experimental values by using a standard cable analysis. Among the cable parameters, a relatively low membrane resistance (Rm) value is a typical feature of skeletal muscle fibers, due to the high total membrane ionic conductance (gm). The high gm is due to the high permeability of resting sarcolemma to chloride and potassium ions, through specific channels open at resting membrane potential. In particular the total gm of EDL myofibers is due for the 80% to the chloride channel conductance (gCl) of ClC-1 chloride channels, while the remaining 20% is due to potassium conductance of different potassium channel subsets. An increase in Rm and a significant decrease of gm (mostly due to a decrease in gCl) are typical cellular hallmark of mdx diaphragm and exercised EDL muscle. The decrease in gm is related to complex mechanisms involving both expression and biochemical modulation of ClC-1 channels during muscle degeneration. A decrease in gm is considered a cellular marker of tissue sufferance.

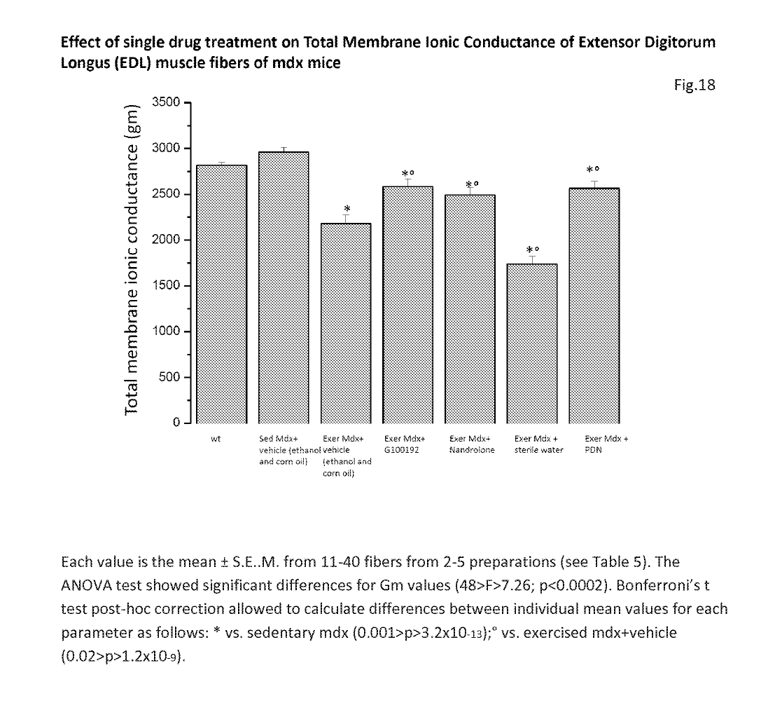

In the present study, higher values of Rm in EDL myofibers of exercised mdx mice vs. sedentary mdx and weight mice, in the absence of any change of resting membrane potential was observed (FIG. 18). A slight difference was observed in the Rm and gm values of the two vehicle-treated groups of mdx mice. All the drug treatments lead to a significant decrease of Rm paralleled by an increase in gm. The effect was particularly evident with Compound (I) which produced effects comparable to that of PDN. PDN produced an effect consistent with that observed in previous studies (De Luca et al., JPET 2003).

### Biochemical Markers: Effect of the Treatment on Creatine Kinase, Lactate Dehydrogenase and Reactive Oxygen Species

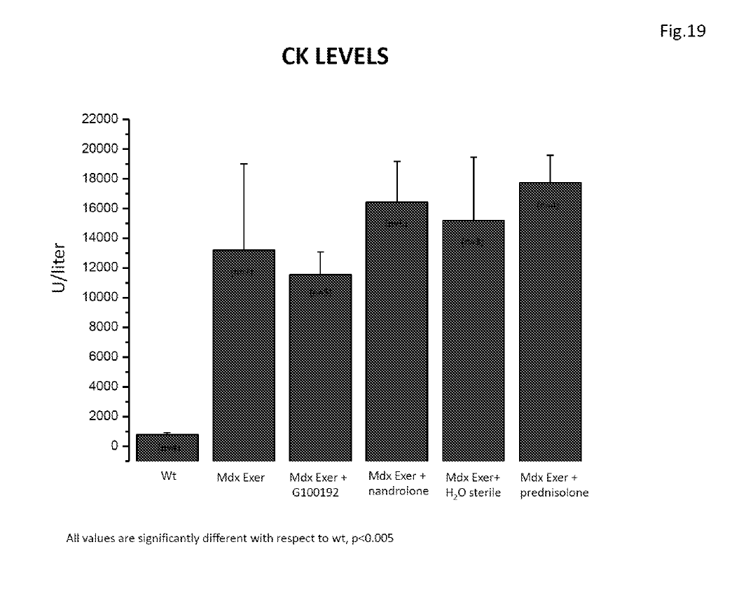

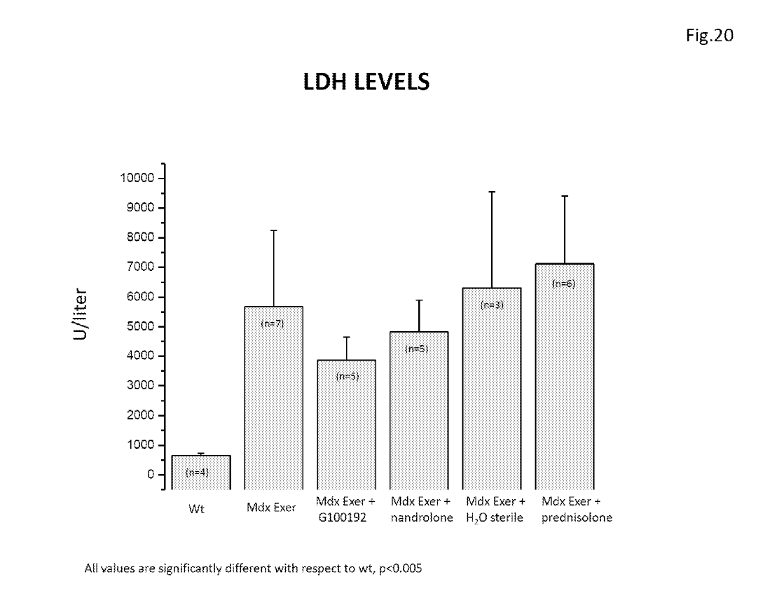

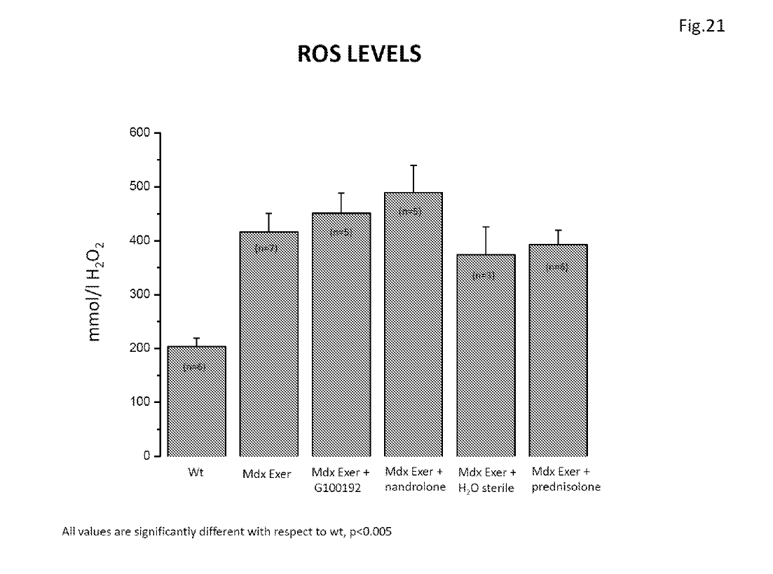

A marked elevation of plasma creatine kinase is a typical diagnostic marker of muscular dystrophy. In parallel an increase in lactate dehydrogenase is also observed as a sign of metabolic sufferance while an increase on reactive oxygen species can occur as a result of ongoing oxidative stress. Generally, these biochemical indices are further aggravated by the exercise protocol. However, in the present study all the three parameters CK, LDH and ROS were particularly altered in the sedentary mdx mice and therefore no remarkable effect of exercise was observed. The effects observed with the various drug treatment is shown in FIGS. 19-21. As can be seen no significant amelioration was observed with any of the drug used on any of these biochemical markers. A slight, but not significant reduction of LDH was observed in Compound (I)-treated mdx mice. This confirms the lack of effect of PDN on CK and LDH that has been already found in previous studies.

### Histology and Morphometry

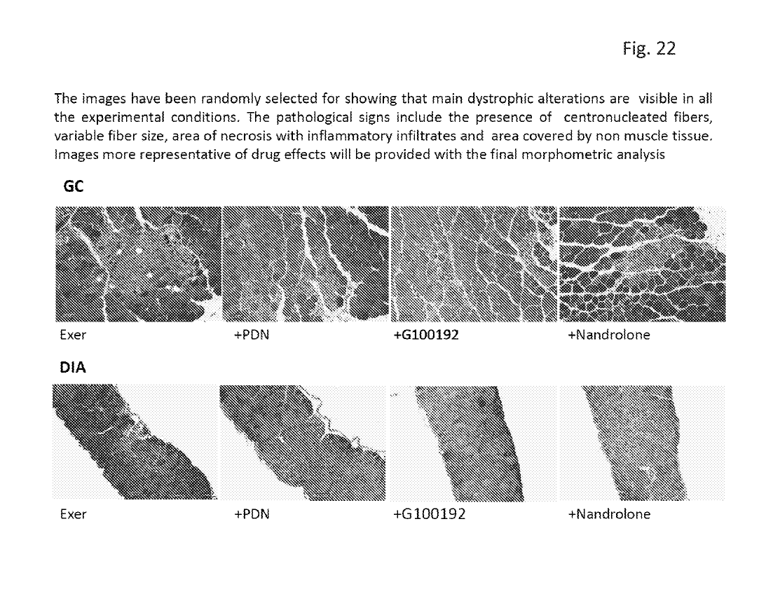

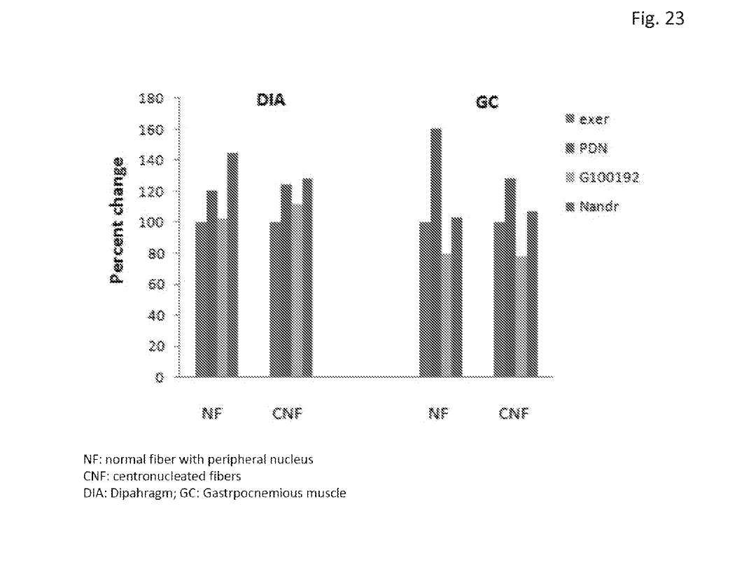

Representative pictures of histology profile of diaphragm and GC muscles in the various experimental conditions are shown in FIG. 22. Both muscles showed the typical dystrophic features, such as the alteration of the muscle architecture, with the presence of area of necrosis, infiltrates and large non-muscle area, likely due to deposition of fibrotic and adipose tissue. A large variability in fiber size and the presence of centronucleated fibers (CNF) were also clearly detectable. The alterations were still present in the groups of treated muscles, although some qualitative signs of amelioration could be observed. A preliminary morphometric analysis on a restricted number of sections suggested no change in the percentage of centronucleated fibers and slight reduction in necrosis and/or in non-muscle area in diaphragm and GC muscle of drug-treated animals. An increase in fiber area of both normal and centronucleated fibers in nandrolone and PDN, but not in Compound (I), treated muscle has been also observed (FIG. 23).

### Example 2

### Comparison of Treatment of Mdx Mice with Compound (I), Nandrolone, and α-Methylprednisolone

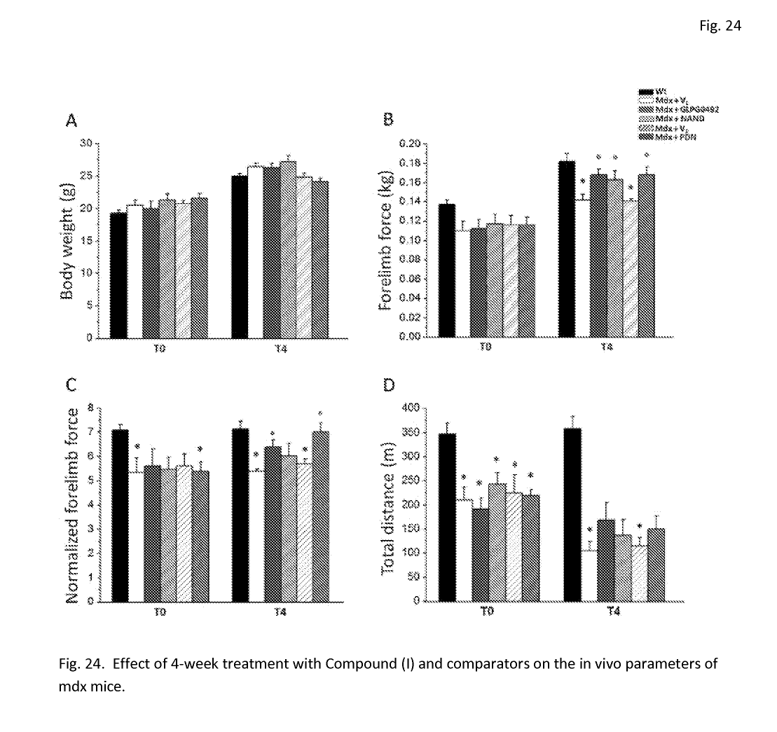

Compound (I), nandrolone, and α-methylprednisolone were given 6 days per week to wild-type (Wt) and mdx mice. FIG. 24 shows in vivo parameters at the beginning (T0) and after 4 (T4) weeks of the protocol for wild-type (Wt) and mdx mice treated either with corn oil (Mdx+V1) or with 30 mg/kg composition comprising Compound (I) (Mdx+Compound (I)), 5 mg/kg nandrolone (Mdx+NAND), water (Mdx+V2) or 1 mg/kg α-methylprednisolone (Mdx+PDN). In each graph, the bars are the means±S.E.M. from 5 to 7 animals. Significant differences between groups were evaluated using the ANOVA test for multiple comparisons and the Bonferroni t-test post hoc correction.

In (A), the bars show the body weight values (body weight) in g. No significant differences were observed between the values of mdx mice (either treated or not) using an ANOVA test. In (B), the bars show the maximal forelimb strength (Forelimb force) in kg. The ANOVA test did not indicate any significant differences at time 0 (T0). A significant difference was found at time 4 (T4) (F>5.79; p<0.005). The post hoc Bonferroni t-test results are indicated as follows: *significantly different with respect to Wt mice with p<0.003; ° significantly different with respect to respective vehicle-treated mdx exercised mice with 0.007<p<0.01. In (C), the bars show the normalised fore limb force values (Normalised forelimb force) calculated by normalising for each mouse the fore limb strength to the respective body weight. The ANOVA test did not show significant difference for time 0 (T0). A significant difference was found for time 4 (T4) (F>5.8; p<0.006). The post hoc Bonferroni t-test results are as follows: *significantly different with respect to Wt mice with 0.0006<p<0.03; ° significantly different with respect to mdx exercised mice with 0.003<p<0.02. In (D), the total distance (in m) is shown for running in a treadmill exhaustion test. All values were significantly different with respect to wt animals at both T0 and T4. The post hoc Bonferroni t-test results are indicated as follows: *significantly different with respect to wt mice with 5.5×10⁻⁷ <p<0.01.

### Example 3

### Treatment of Mdx Mice with Various Amounts of Compound (I)

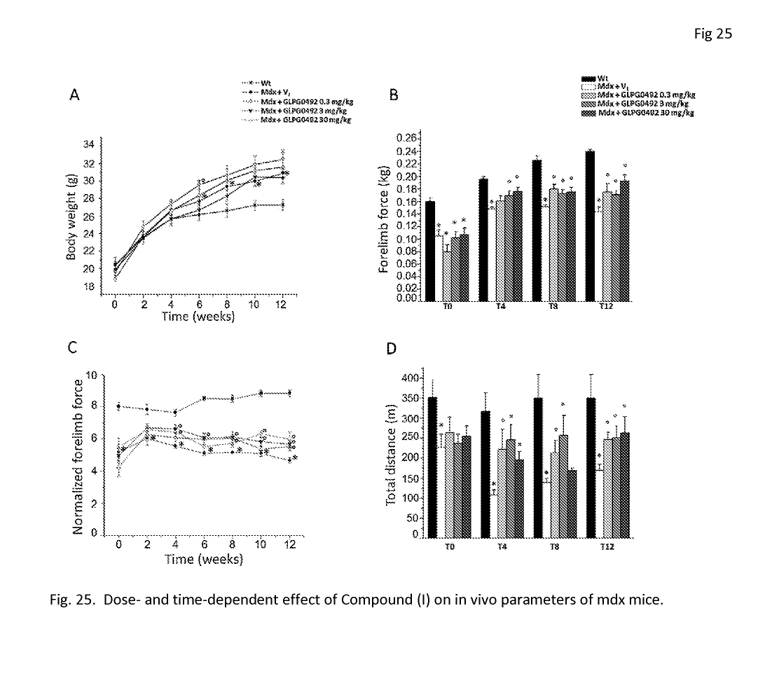

Compound (I) was given 6 days per week to wild-type (Wt) and mdx mice. FIG. 25 indicates that at various time points, from the beginning (T0) up to 12 weeks of protocol (T12), the in vivo parameters of wild-type (Wt) and mdx mice treated with corn oil (Mdx+V1) or with Compound (I) (Mdx+Compound (I)) at 0.3, 3 and 30 mg/kg. In each graph, the values, as the means±S.E.M., from 5 to 8 animals are indicated. The significant differences between groups were evaluated using an ANOVA test for multiple comparison and the Bonferroni t-test post hoc correction.

In (A), the bars indicate the body weight values (Body weight) in g. The ANOVA test did not indicate a significant difference for BW at time 0, time 4 and time 6. A significant difference was found for BW at time 8 (F>3.9; p<0.02) and time 12 (F>3.8; p<0.03). The post hoc Bonferroni t-test results are indicated as follows: *significantly different with respect to Wt mice with 0.006<p<0.01 and ° significantly different with respect to mdx exercised mice with p<0.05. In (B), the bars indicate the maximal forelimb strengths (forelimb force), in kg at either the beginning (Fmax T0), the 4th (Fmax T4), 8th (Fmax T8) and the 12th (Fmax T12) week of the protocol. The ANOVA test indicated a significant difference at T0 (F>9; p<0.0006), T4 (F>11; p<0.0002), T8 (F>3.76; p<0.02) and T12 (F>5.4; p<0.006). The post hoc Bonferroni t-test results are indicated as follows: *significantly different with respect to wt mice with 9.3×10⁻⁸<p<0.02 and ° significantly different with respect to mdx exercised mice with 3.6×10⁻⁶<p<0.01. In (C), the normalised forelimb force values (normalised forelimb force) were calculated by normalising, for each mouse, the forelimb strength to the respective body weight. The ANOVA test indicated significant differences at all time points from T4 onward (F>4; p<0.02). The post hoc Bonferroni t-test results are indicated as follows: *significantly different with respect to wt mice with p<0.0005 and ° significantly different with respect to mdx exercised mice with 0.002<p<0.02. In (D), the total distance (in m) run in an exhaustion test on the treadmill is shown. Significant differences between groups were evaluated by ANOVA test and Student's t test. All values are significantly different with respect to wt animals at corresponding time point. A significant difference was found at T4 (F>5.4; p<0.007), T8 (F>4; p<0.02), and T12 (F>5; p<0.009). The post hoc Bonferroni t-test results are indicated as follows: *significantly different with respect to wt mice with 0.0009<p<0.02 and ° significantly different with respect to mdx exercised mice with 0.009<p<0.03.

### Example 4

### Effect of Treatment with Compound (I), Nandrolone, and α-Methylprednisolone on Androgen-Sensitive and Other Potential Target Tissues

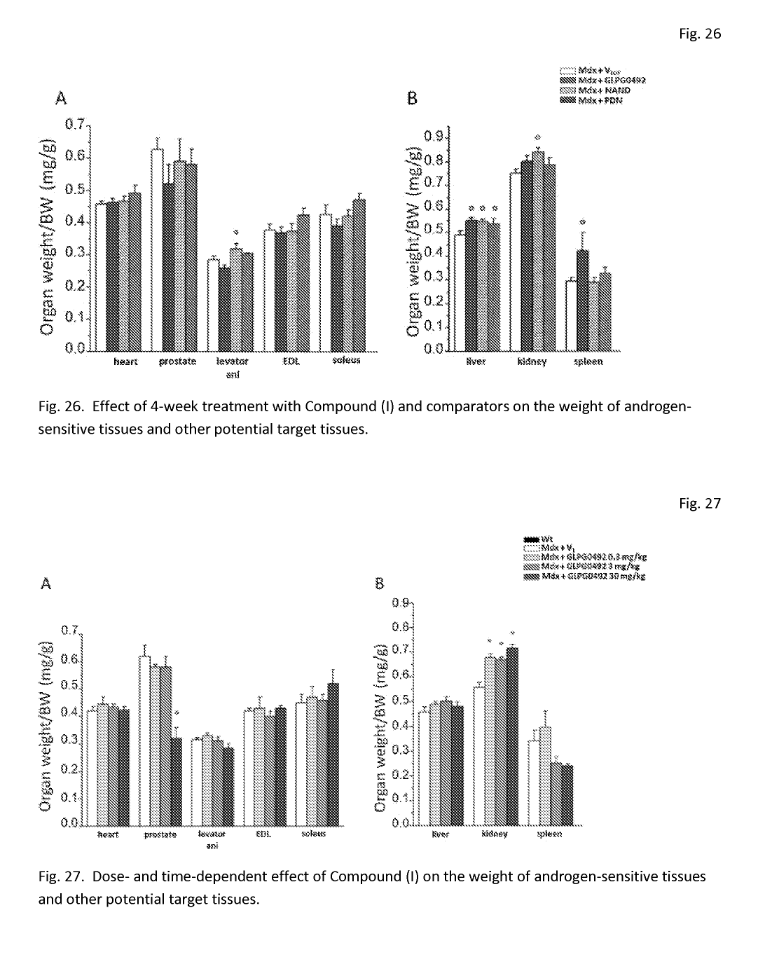

Treatment of mdx mice with Compound (I), nandrolone, and α-methylprednisolone was given for 6 days per week. FIG. 26 shows the effect of a 4-week treatment with Compound (I) and comparators on the weight of androgen-sensitive tissues and other potential target tissues. Each bar represents the mean±S.E.M. from 5 to 10 animals and shows the tissue mass normalised with respect to the individual body weight of mdx mice treated with either vehicle (corn oil and water; Mdx+V_{TOT}) or with 30 mg/kg Compound (I) (Mdx+Compound (I)), 5 mg/kg nandrolone (Mdx+NAND) or 1 mg/kg α-methylprednisolone (Mdx+PDN).

In (A), the figure shows the weight of androgen-sensitive tissues, i.e., the heart, prostate, levator ani, EDL and soleus muscles. The normalised values for the levator ani have been scaled by a factor of ten for graphical reasons. The ANOVA analysis and Bonferroni t test indicated significant differences only for the levator ani weight (F>4; p<0.015). ° Significantly different vs. mdx vehicle-treated (p<0.05). In (B), the figure shows the weights of the spleen, liver and kidneys. The normalised values for the liver have been scaled by a factor of ten for graphical reasons. An ANOVA analysis and the Bonferroni t test indicated significant differences only for liver weight (F>3; p<0.04); ° significantly different vs. mdx vehicle-treated (p<0.02).

### Example 5

### Dose- and Time-Dependent Effect Treatment on Androgen-Sensitive Tissues and Other Potential Target Tissues

The dose- and time-dependent effect of Compound (I) on the weight of androgen-sensitive tissues and other potential target tissues is shown on FIG. 27. Each bar represents the mean±S.E.M. from 5 to 8 animals and show the tissue mass normalised with respect to the individual body weight of mdx mice treated with either corn oil (Mdx+V₁) or with Compound (I) at 0.3, 3 or 30 mg/kg (Mdx+Compound (I)). The drugs were given 6 days per week. In (A), the figure shows the weights of androgen-sensitive tissues, i.e., heart, prostate, levator ani, EDL and soleus muscles. The normalised values for the levator ani have been scaled by a factor of ten for graphical reasons. An ANOVA analysis and the Bonferroni t test indicated significant differences only for prostate weight (F>12; p<5.4×10⁻⁵); ° significantly different vs. mdx vehicle-treated (p<1.4×10⁻⁵). In (B), the figure shows the weight of the spleen, liver and kidneys. The normalised values for the liver have been scaled by a factor of ten for graphical reasons. An ANOVA analysis and Bonferroni t test indicated significant differences only for kidney weight (F>19; p<1.9×10⁻⁶); ° significantly different vs. mdx vehicle-treated (p<0.03).

### Example 6

### Effect of Various Drug Treatments on the Maximal Isometric Twitch and Tetanic Tension of the Diaphragm

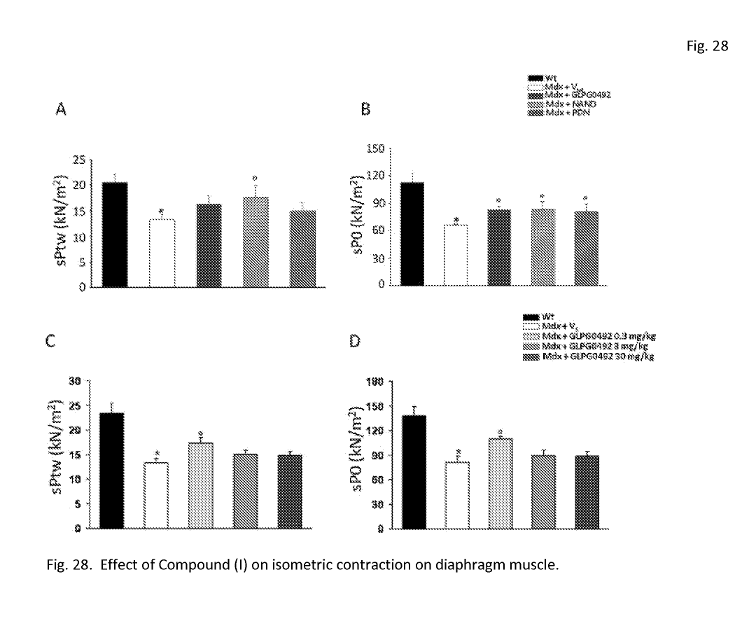

FIG. 28, (A) and (B) list the normalised values of the maximal isometric twitch (sP_{tw} measured in kN/m²) and tetanic tension (sP₀ measured in kN/m²) of the diaphragm strips from wt and mdx mice, treated or not, from the first study. The figures list the following groups: wild-type mice (Wt) and mdx mice treated with vehicle (water or corn oil: Mdx+V_{TOT}), 30 mg/kg Compound (I) (Mdx+Compound (I)), 5 mg/kg nandrolone (Mdx+NAND) or 1 mg/kg α-methylprednisolone (Mdx+PDN). The drugs were given 6 days per week. Each bar is the mean±S.E.M. for 4-7 animals per group. The significant differences between groups were evaluated by ANOVA test for multiple comparison (F values) as follows: F=3; p<0.05. The Bonferroni t-test post hoc correction was used to estimate significant differences between individual mean values and are indicated as follows: *significant difference vs wt (0.001<p<0.05); ° significant difference vs. Mdx+V_{TOT} (p<0.01). In (C) and (D), the normalised values of the maximal isometric twitch (sP_{tw} measured in kN/m²) and tetanic tension (sP₀ measured in kN/m²) of diaphragm strips from WT and mdx mice, treated or not, belonging to the second study are shown. The figures show the wild-type mice (Wt) and mdx mice treated with vehicle (only corn oil: mdx+V1) or with Compound (I) at 0.3, 3 or 30 mg/kg (mdx+Compound (I)). The drugs were given 6 days per week. Each bar represents the mean S.E.M. for 4-7 animals per group. The significant difference between groups was evaluated by the ANOVA test for multiple comparisons (F values) as follows: F=3; p<0.05. A Bonferroni t-test post hoc correction was used and the results are indicated as follows: *significant difference vs wt (0.001<p<0.05) and ° significant difference vs Mdx+V1 (p<0.01).

### Example 7

### Effect of Treatment on Contractile Parameters of Isolated EDL Muscles

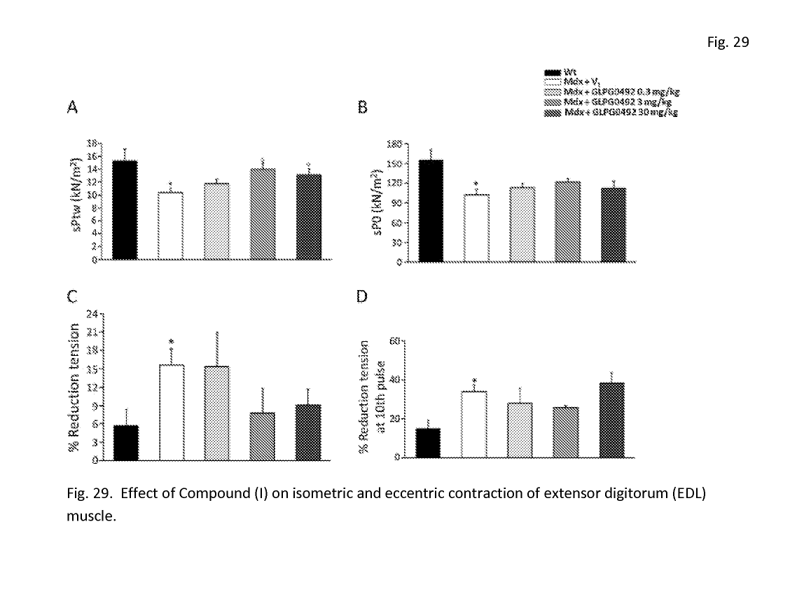

The contractile parameters of the isolated EDL muscles from wt and mdx mice treated 6 days per week with either corn oil (Mdx+V1) or with Compound (I) at 0.3, 3 or 30 mg/kg (Mdx+Compound (I)) is shown in FIG. 29.

In (A), the normalised values for maximal isometric twitch (sP_{tw} measured in kN/m²) are shown. ANOVA test indicated significant differences with F=4 and p<0.05. The post hoc Bonferroni t-test results are indicated as follows: *significant difference vs. wt (p<0.05) and° vs Mdx+V1 (0.005<p<0.05). In (B), the normalised values of maximal isometric tetanic tension (sP₀ measured in kN/m²) are shown. ANOVA test indicated significant differences with F=4 and p<0.03. The post hoc Bonferroni t-test results are indicated as follows: *significant difference vs wt (0.01<p<0.05). In (C), the muscle fatigue, defined as the percentage drop of force at the 10th pulse with respect to the first contraction, is shown. No significant difference was observed as evaluated with ANOVA. A Bonferroni t-test indicated significant differences, and the results are indicated as follows: *significant difference vs wt (p<0.005). In (D), the percentage of tension reduction during eccentric contraction (calculated as the drop at the 10th pulse vs the tension at the first eccentric stimulus) is shown. An ANOVA test indicated significant differences with F=4 and p<0.02. The post hoc Bonferroni t-test results are indicated as follows: *significant difference vs wt (p<0.05). Each bar represents the mean S.E.M. for 4-7 animals per group.

### Example 8

### Comparison of the Mechanical Threshold in Mdx Treated with Various Drugs

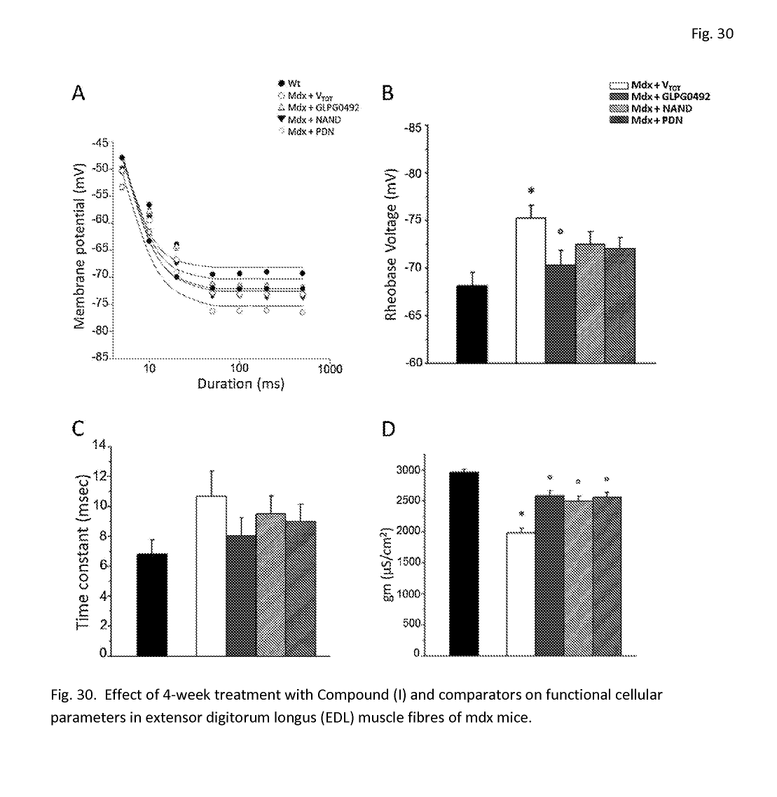

In FIG. 30(A), the data, expressed as the means±S.E.M. from 14 to 30 values from 2 to 5 preparations, show the voltages for the contraction of EDL myofibres (mechanical threshold) at increasing pulse duration in wild-type mice (WT, black circles) and in mdx mice treated with either vehicle (corn oil and water; Mdx+V_{TOT}, white circles), 30 mg/kg Compound (I) (white triangles), 5 mg/kg nandrolone (upside-down black triangles) or 1 mg/kg PDN (white rhombus). The drugs were given 6 days per week. The voltage threshold values of myofibres of mdx mice treated with 30 mg/kg Compound (I), 5 mg/kg nandrolone or 1 mg/kg PDN were significantly more positive with respect to those of mdx mice treated with vehicle (p<0.03 or less by Student's t test) at each pulse duration. For some data points, the standard error bar is not visible because it is smaller than the symbol size. In (B) and (C), the rheobase voltage, in mV and the time constant, in ms, with relative standard errors, have been calculated from the fit of data points of the voltage-duration curves in A. In (D), the total resting membrane ionic conductances (gₘ) in μS/cm² of EDL muscle fibres of the same experimental groups described in A are shown. The bars represent the means±SEM from the number of 3-5 prep/25-37 fibres. For each parameter, the significant differences between groups were evaluated using ANOVA for multiple comparisons (F values) and the Bonferroni t-test post hoc correction. Significant differences were found for rheobase voltage (F>4; p<0.003) and gₘ (F>7; p<0.0002). The post hoc Bonferroni t-test results are indicated as follows: *significantly different with respect to wt mice with p<0.05 and ° significantly different with respect to mdx exercised mice with p<0.02.

### Example 9

### Comparison of Mechanical Threshold for Treated and Untreated Mdx Mice

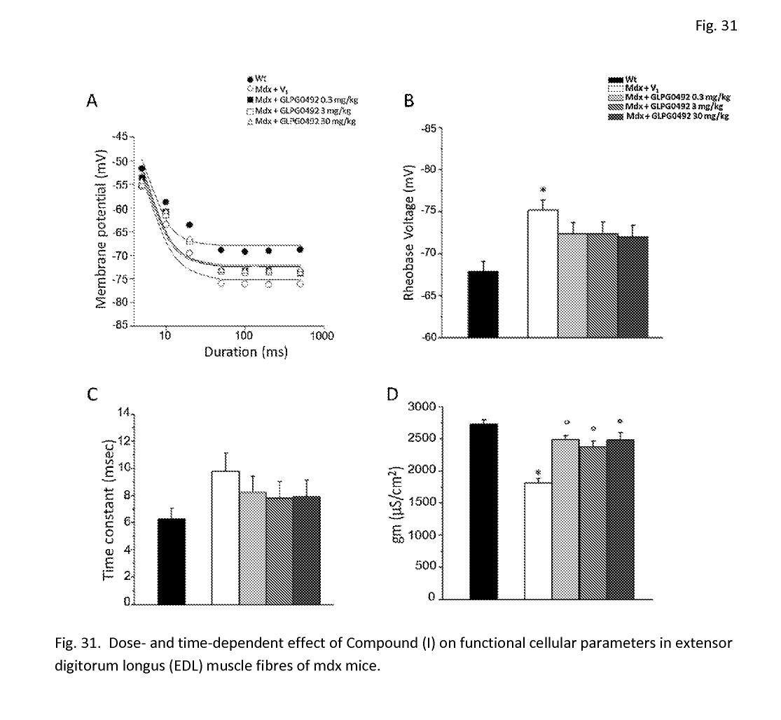

In FIG. 31(A), the data, expressed as the means±S.E.M. from 27 to 41 values from 3 preparations, show the voltages for the contraction of EDL myofibres (mechanical threshold) at increasing pulse duration in wild type mice (WT, black circles) and in mdx mice treated with either corn oil (Mdx+V1, white circles) or Compound (I) at 0.3 (white square), 3 (black square) or 30 mg/kg (white triangles). The drugs were given 6 days per week. The voltage threshold values of the myofibres of mdx mice treated with Compound (I) at any dose were significantly more positive with respect to those of mdx mice treated with vehicle (p<0.01 or less by Student's t test). For some data points, the standard error bar is not visible because it is smaller than the symbol size. In (B) and (C), the rheobase voltages, in mV and time constant, in ms, with relative standard errors, respectively, have been calculated from the fit of data points of the voltage-duration curves in A. In (D), the total resting membrane ionic conductances (gₘ) in μS/cm² of EDL muscle fibres of the same experimental groups described in A are shown. The bars represent the means±SEM from the values of 2-3 prep/21-41 fibres. For each parameter, the significant differences between groups were evaluated using an ANOVA test for multiple comparisons (F values) and the Bonferroni t-test post hoc correction. Significant differences were found for rheobase voltage (F>4; p<0.003) and gₘ (F>22; p<1.3×10⁻⁶). The post hoc Bonferroni t-test results are indicated as follows: *significantly different with respect to wt mice with 1.1×10⁻¹³<p<0.02 and ° significantly different with respect to mdx exercised mice with p<1×10⁻⁶.

### Example 10

### Histology of Diaphragm and Gastrocnemius Muscles after Treatment with Compound (I)

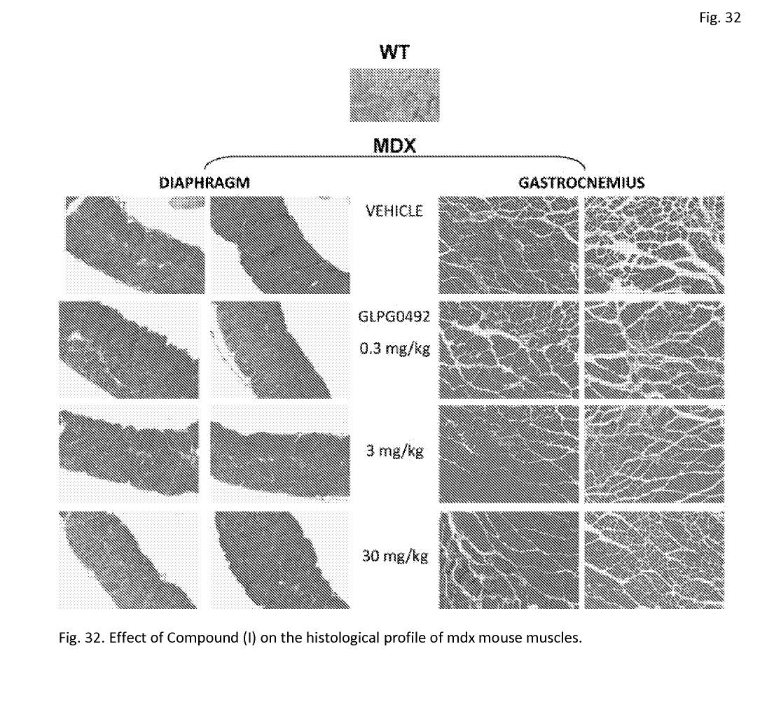

Haematoxylin-eosin staining showing the morphological profiles of diaphragm (DIA) and gastrocnemius (GC) muscles from mdx mice either untreated (Vehicle) or treated with GPL0492 at different dosages (0.3, 3, and 30 mg/kg) is shown on FIG. 32. The drugs were given 6 days per week. For qualitative comparison, a typical profile of a wt GC muscle is shown at the top of the figure. The sections show the poorly homogenous structure of dystrophic muscle, with great variability in fibre dimension, large areas of necrosis accompanied by mononuclear infiltrates and/or small regenerating fibres. The areas of non-muscle tissue are also visible. The images are at 20× magnification.

### Example 11

### Effect of Compound (I), Nandrolone, or α-Methylprednisolone on Fibrosis Markers

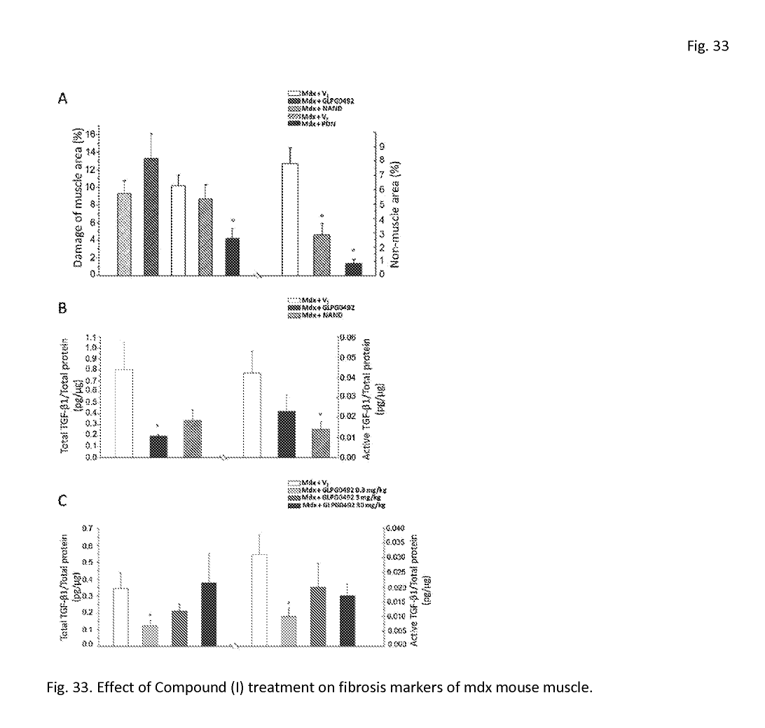

Mdx mice were treated either with corn oil (Mdx+V1) or with 30 mg/kg Compound (I) (Mdx+Compound (I)), 5 mg/kg nandrolone (Mdx+NAND), water (Mdx+V2) or 1 mg/kg α-methylprednisolone (Mdx+PDN) for 6 days per week. FIG. 33(A) depict the percentage of area of muscle damage (left) and the percentage of non-muscle area (right) of diaphragm muscle, as measured by haematoxylin-eosin staining. Each bar is the mean of at least 3 muscles/approximately 10 fields per muscle. Significant differences between groups were evaluated using an ANOVA test, and the Bonferroni t-test post hoc correction. The results are indicated as follows: ° significantly different with respect to mdx mice treated with corn oil p<0.03. In (B), the bars show the levels of total (left) and active TGF-β1 (right), in diaphragm muscle, in mdx mice treated with either vehicle (corn oil, Mdx+V1) 30 mg/kg Compound (I) (Mdx+Compound (I)), or 5 mg/kg nandrolone (Mdx+NAND), as measured by ELISA. Each value is the mean±S.E.M. from 4 to 5 preparations. An ANOVA test for multiple comparisons between the groups did not indicate any significant difference in TGF-β1 levels. The post hoc Bonferroni t-test results are indicated as follows: ° significantly different with respect to vehicle-treated mdx mice, p<0.03. In (C), the bars show the levels of total (left) and active TGF-β1 (right) in diaphragm muscle for mdx mice treated with either corn oil (Mdx+V1), or with Compound (I) at 0.3, 3 or 30 mg/kg (Mdx+Compound (I)), as measured by ELISA. The drugs were given 6 days per week. Each value is the mean±S.E.M. from 4 to 5 preparations. Significant differences between groups were evaluated using Student's t test. ° Significantly different with respect to mdx exercised mice 0.05<p<0.025.

### Example 12

### Plasma Levels of Compound (I) after Subcutaneous Injection

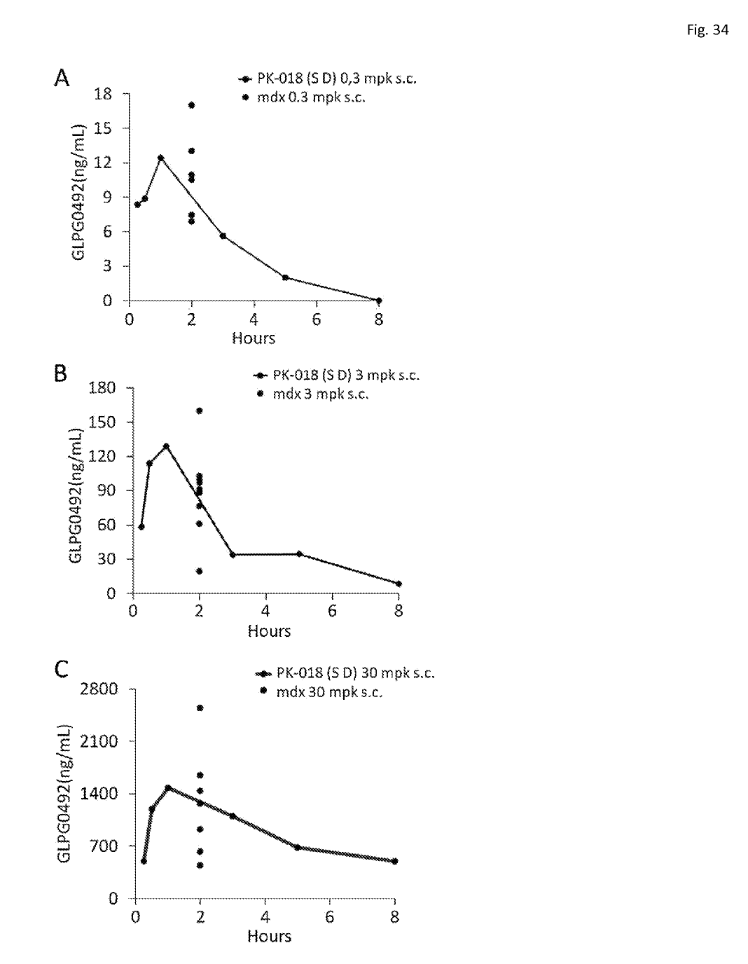

FIG. 34 shows Compound (I) plasma levels assessed over an 8-h period after s.c. delivery of 0.3 mg/kg (A), 3 mg/kg (B), or 30 mg/kg (C) of the compound into wild-type mice receiving a single acute dose (black circle with a slash) or into mdx mice receiving chronic dosing (black circle).

### Example 13

### Comparison of Testosterone Levels in Wild-Type, Exercised and not-Exercised Mdx Mice

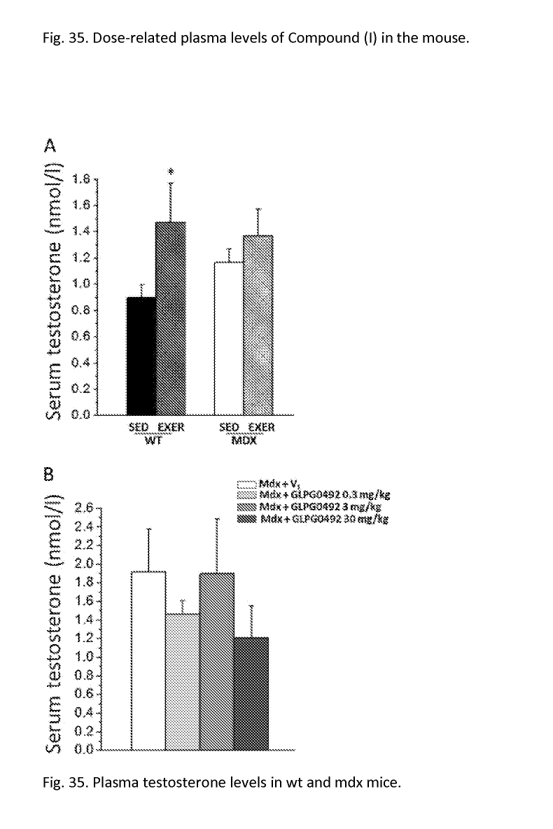

In FIG. 35(A), the bars show the serum testosterone levels of 8-week-old wild type and mdx mice either exercised for 4 weeks (WT EXER; MDX EXER) or not (WT SED; MDX SED). Each bar is the mean±S.E.M. from 5 to 6 animals. Significant differences between groups were evaluated by Student's t test. *Significantly different with respect to wt mice with p<0.05. In (B), the bars show the effect of Compound (I) on plasma testosterone levels in mdx mice. Each bar is the mean±S.E.M. from 5 to 7 animals.

### Example 14

### Effect of Compound (I) Treatment on ICF-1 and Follistatin Gene Levels

Real-time PCR analysis was performed for insulin-like growth factor-1 (IGF-1) and follistatin, genes involved in the control of muscle mass; myogenin, a marker of muscle regeneration; and peroxisome proliferator receptor γ-coactivator (PGC)-1α, a modulator of muscle metabolism and of mechano-transduction signalling.

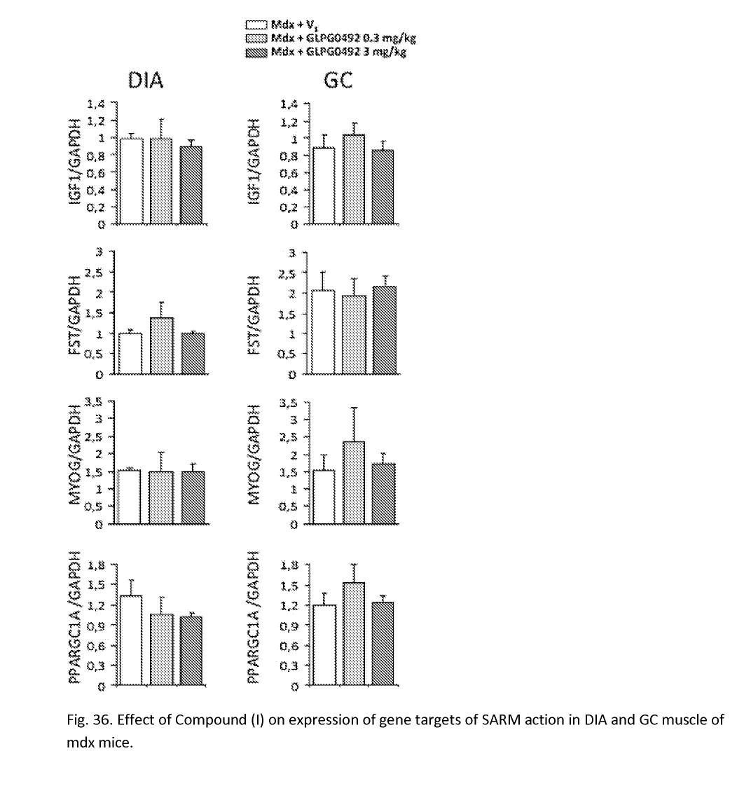

FIG. 36 shows the normalised values of the target genes with respect to a housekeeping gene (GADPH) for vehicle (Mdx+V1); 0.3 mg/kg Compound (I) (Mdx+0.3 mg/kg Compound (I)) and 3 mg/kg Compound (I) (Mdx+3 mg/kg Compound (I)) in diaphragm (left side; DIA) and gastrocnemius (right side; GC). The drugs were given 6 days per week. Each value is the mean±S.E.M. from 4 to 5 preparations.

### REFERENCES

Alman, B. A., Raza, S. N. and Biggar, W. D. (2004). “Steroid treatment and the development of scoliosis in males with duchenne muscular dystrophy.” J Bone Joint Surg Am 86-A(3): 519.
Ansved, T. (2003). “Muscular dystrophies: influence of physical conditioning on the disease evolution.” Curr Opin Clin Nutr Metab Care 6(4): 435.
Bach, J. R., Ishikawa, Y. and Kim, H. (1997). “Prevention of pulmonary morbidity for patients with Duchenne muscular dystrophy.” Chest 112(4): 1024.
Barton-Davis, E. R., Cordier, L., Shoturma, D. I., et al. (1999). “Aminoglycoside antibiotics restore dystrophin function to skeletal muscles of mdx mice [see comments].” J Clin Invest 104(4): 375.
Baydur, A., Layne, E., Aral, H., et al. (2000). “Long term non-invasive ventilation in the community for patients with musculoskeletal disorders: 46 year experience and review.” Thorax 55(1): 4.
Beggs, A. H., Koenig, M., Boyce, F. M., et al. (1990). “Detection of 98% of DMD/BMD gene deletions by polymerase chain reaction.” Hum Genet 86(1): 45.
Bernasconi, P., Torchiana, E., Confalonieri, P., et al. (1995). “Expression of transforming growth factor-beta 1 in dystrophic patient muscles correlates with fibrosis. Pathogenetic role of a fibrogenic cytokine.” J Clin Invest 96(2): 1137.
Biggar, W. D., Politano, L., Harris, V. A., et al. (2004). “Deflazacort in Duchenne muscular dystrophy: a comparison of two different protocols.” Neuromuscul Disord 14(8-9): 476.
Brook, P. D., Kennedy, J. D., Stern, L. M., et al. (1996). “Spinal fusion in Duchenne's muscular dystrophy.” J Pediatr Orthop 16(3): 324.
Brooks, A. P. and Emery, A. E. (1977). “The incidence of Duchenne muscular dystrophy in the South East of Scotland.” Clin Genet 11(4): 290.
Bushby, K., Muntoni, F. and Bourke, J. P. (2003). “107th ENMC international workshop: the management of cardiac involvement in muscular dystrophy and myotonic dystrophy. 7th-9th June 2002, Naarden, the Netherlands.” Neuromuscul Disord 13(2): 166.
Campbell, C. and Jacob, P. (2003). “Deflazacort for the treatment of Duchenne Dystrophy: a systematic review.” BMC Neurology 3(1): 7.
Chen, Y. W., Zhao, P., Borup, R., et al. (2000). “Expression profiling in the muscular dystrophies: identification of novel aspects of molecular pathophysiology.” J Cell Biol 151(6): 1321.
Corrado, G., Lissoni, A., Beretta, S., et al. (2002). “Prognostic value of electrocardiograms, ventricular late potentials, ventricular arrhythmias, and left ventricular systolic dysfunction in patients with Duchenne muscular dystrophy.” Am J Cardiol 89(7): 838.
den Dunnen, J. T., Bakker, E., Breteler, E. G., et al. (1987). “Direct detection of more than 50% of the Duchenne muscular dystrophy mutations by field inversion gels.” Nature 329(6140): 640.
Den Dunnen, J. T., Grootscholten, P. M., Bakker, E., et al. (1989). “Topography of the Duchenne muscular dystrophy (DMD) gene: FIGE and cDNA analysis of 194 cases reveals 115 deletions and 13 duplications.” Am J Hum Genet 45(6): 835.
Dent, K. M., Dunn, D. M., von Niederhausern, A. C., et al. (2005). “Improved molecular diagnosis of dystrophinopathies in an unselected clinical cohort.” Am J Med Genet A.
Do, T. (2002). “Orthopedic management of the muscular dystrophies.” Curr Opin Pediatr 14(1): 50.
Eagle, M., Baudouin, S. V., Chandler, C., et al. (2002). “Survival in Duchenne muscular dystrophy: improvements in life expectancy since 1967 and the impact of home nocturnal ventilation.” Neuromuscul Disord 12(10): 926.
Escolar, D, . . . (2011). Randoized controlled blinded trial of daily vs. weekly prednisone treament in Ducehnne Muscular Dystrophy. Neurology, in press.
Fenichel, G. M., Florence, J. M., Pestronk, A., et al. (1991). “Long-term benefit from prednisone therapy in Duchenne muscular dystrophy.” Neurology 41(12): 1874.
Finder, J. D., Birnkrant, D., Carl, J., et al. (2004). “Respiratory care of the patient with Duchenne muscular dystrophy: ATS consensus statement.” Am J Respir Crit Care Med 170(4): 456.
Finsterer, J. and Stollberger, C. (2003). “The heart in human dystrophinopathies.” Cardiology 99(1): 1.
Gillard, E. F., Chamberlain, J. S., Murphy, E. G., et al. (1989). “Molecular and phenotypic analysis of patients with deletions within the deletion-rich region of the Duchenne muscular dystrophy (DMD) gene.” Am J Hum Genet 45(4): 507.
Systemic administration of PRO051 in Duchenne's muscular dystrophy.
Goemans N M, Tulinius M, van den Akker J T, Burm B E, Ekhart P F, Heuvelmans N, Holling T, Janson A A, Platenburg G J, Sipkens J A, Sitsen J M, Aartsma-Rus A, van Ommen G J, Buyse G, Darin N, Verschuuren J J, Campion G V, de Kimpe S J, van Deutekom J C.
N Engl J Med. 2011 Apr. 21; 364(16):1513-22. Epub 2011 Mar. 23.
Gomez-Merino, E. and Bach, J. R. (2002). “Duchenne muscular dystrophy: prolongation of life by noninvasive ventilation and mechanically assisted coughing.” Am J Phys Med Rehabil 81(6): 411.
Gozal, D. (2000). “Pulmonary manifestations of neuromuscular disease with special reference to Duchenne muscular dystrophy and spinal muscular atrophy.” Pediatr Pulmonol 29(2): 141.
Griggs, R. C., Moxley, R. T. d., Mendell, J. R., et al. (1991). “Prednisone in Duchenne dystrophy. A randomized, controlled trial defining the time course and dose response. Clinical Investigation of Duchenne Dystrophy Group.” Arch Neurol 48(4): 383.
Hoffman, E. P., Arahata, K., Minetti, C., et al. (1992). “Dystrophinopathy in isolated cases of myopathy in females.” Neurology 42(5): 967.
Hoffman, E. P., Brown, R. H., Jr. and Kunkel, L. M. (1987a). “Dystrophin: the protein product of the Duchenne muscular dystrophy locus.” Cell 51(6): 919.
Hoffman, E. P., Fischbeck, K. H., Brown, R. H., et al. (1988). “Characterization of dystrophin in muscle-biopsy specimens from patients with Duchenne's or Becker's muscular dystrophy.” N Engl J Med 318(21): 1363.
Hoffman, E. P., Knudson, C. M., Campbell, K. P., et al. (1987b). “Subcellular fractionation of dystrophin to the triads of skeletal muscle.” Nature 330(6150): 754.
Hoffman, E. P. and Kunkel, L. M. (1989). “Dystrophin abnormalities in Duchenne/Becker muscular dystrophy.” Neuron 2(1): 1019.
Jeppesen, J., Green, A., Steffensen, B. F., et al. (2003). “The Duchenne muscular dystrophy population in Denmark, 1977-2001: prevalence, incidence and survival in relation to the introduction of ventilator use.” Neuromuscul Disord 13(10): 804.
Koenig, M., Hoffman, E. P., Bertelson, C. J., et al. (1987). “Complete cloning of the Duchenne muscular dystrophy (DMD) cDNA and preliminary genomic organization of the DMD gene in normal and affected individuals.” Cell 50(3): 509.
Kunkel, L. M. (1986). “Analysis of deletions in DNA from patients with Becker and Duchenne muscular dystrophy.” Nature 322(6074): 73.
Kunkel, L. M., Monaco, A. P., Middlesworth, W., et al. (1985). “Specific cloning of DNA fragments absent from the DNA of a male patient with an X chromosome deletion.” Proc Natl Acad Sci USA 82(14): 4778.
Lesca, G., Demarquay, G., Llense, S., et al. (2003). “[Symptomatic carriers of dystrophinopathy with chromosome X inactivation bias].” Rev Neurol (Paris) 159(8-9): 775.
Lim, J. H., Kim, D. Y. and Bang, M. S. (2004). “Effects of exercise and steroid on skeletal muscle apoptosis in the mdx mouse.” Muscle Nerve 30(4): 456.
Luz, M. A., Marques, M. J. and Santo Neto, H. (2002). “Impaired regeneration of dystrophin-deficient muscle fibers is caused by exhaustion of myogenic cells.” Braz J Med Biol Res 35(6): 691.
Manzur, A. Y., Kuntzer, T., Pike, M., et al. (2004). “Glucocorticoid corticosteroids for Duchenne muscular dystrophy.” Cochrane Database Syst Rev(2): CD003725.
McDonald, C. M. (2002). “Physical activity, health impairments, and disability in neuromuscular disease.” Am J Phys Med Rehabil 81(11 Suppl): S108.
Melacini, P., Fanin, M., Duggan, D. J., et al. (1999). “Heart involvement in muscular dystrophies due to sarcoglycan gene mutations.” Muscle Nerve 22(4): 473.
Melacini, P., Vianello, A., Villanova, C., et al. (1996). “Cardiac and respiratory involvement in advanced stage Duchenne muscular dystrophy.” Neuromuscul Disord 6(5): 367.
Melone, M. A., Peluso, G., Galderisi, U., et al. (2000). “Increased expression of IGF-binding protein-5 in Duchenne muscular dystrophy (DMD) fibroblasts correlates with the fibroblast-induced downregulation of DMD myoblast growth: an in vitro analysis.” J Cell Physiol 185(1): 143.
Mendell, J. R., Buzin, C. H., Feng, J., et al. (2001). “Diagnosis of Duchenne dystrophy by enhanced detection of small mutations.” Neurology 57(4): 645.
Mendell, J. R., Moxley, R. T., Griggs, R. C., et al. (1989). “Randomized, double-blind six-month trial of prednisone in Duchenne's muscular dystrophy [see comments].” N Engl J Med 320(24): 1592.
Gentamicin-induced readthrough of stop codons in Duchenne muscular dystrophy.
Malik V, Rodino-Klapac L R, Viollet L, Wall C, King W, Al-Dahhak R, Lewis S, Shilling C J, Kota J, Serrano-Munuera C, Hayes J, Mahan J D, Campbell K J, Banwell B, Dasouki M, Watts V, Sivakumar K, Bien-Willner R, Flanigan K M, Sahenk Z, Barohn R J, Walker C M, Mendell J R.
Ann Neurol. 2010 June; 67(6):771-80.
Monaco, A. P., Bertelson, C. J., Middlesworth, W., et al. (1985). “Detection of deletions spanning the Duchenne muscular dystrophy locus using a tightly linked DNA segment.” Nature 316(6031): 842.
Morrison, J., Lu, Q. L., Pastoret, C., et al. (2000). “T-cell-dependent fibrosis in the mdx dystrophic mouse.” Lab Invest 80(6): 881.
Moxley, R. T., 3rd, Ashwal, S., Pandya, S., et al. (2005). “Practice parameter: corticosteroid treatment of Duchenne dystrophy: report of the Quality Standards Subcommittee of the American Academy of Neurology and the Practice Committee of the Child Neurology Society.” Neurology 64(1): 13.
Muntoni, F., Gobbi, P., Sewry, C., et al. (1994). “Deletions in the 5′ region of dystrophin and resulting phenotypes.” J Med Genet 31(11): 843.
Nevo, Y., Muntoni, F., Sewry, C., et al. (2003). “Large in-frame deletions of the rod-shaped domain of the dystrophin gene resulting in severe phenotype.” Isr Med Assoc J 5(2): 94.
Nigro, G., Politano, L., Nigro, V., et al. (1994). “Mutation of dystrophin gene and cardiomyopathy.” Neuromuscul Disord 4(4): 371.
Nobile, C., Galvagni, F., Marchi, J., et al. (1995). “Genomic organization of the human dystrophin gene across the major deletion hot spot and the 3′ region.” Genomics 28(1): 97.
Oudet, C., Hanauer, A., Clemens, P., et al. (1992). “Two hot spots of recombination in the DMD gene correlate with the deletion prone regions.” Hum Mol Genet 1(8): 599.
Porter, J. D., Khanna, S., Kaminski, H. J., et al. (2002). “A chronic inflammatory response dominates the skeletal muscle molecular signature in dystrophin-deficient mdx mice.” Hum Mol Genet 11(3): 263.
Porter, J. D., Merriam, A. P., Leahy, P., et al. (2003b). “Dissection of temporal gene expression signatures of affected and spared muscle groups in dystrophin-deficient (mdx) mice.” Hum Mol Genet 12(15): 1813.
Rando, T. A. (2001a). “The dystrophin-glycoprotein complex, cellular signaling, and the regulation of cell survival in the muscular dystrophies.” Muscle Nerve 24(12): 1575.
Rando, T. A. (2001b). “Role of nitric oxide in the pathogenesis of muscular dystrophies: a “two hit” hypothesis of the cause of muscle necrosis.” Microsc Res Tech 55(4): 223.
Rando, T. A. (2002). “Oxidative stress and the pathogenesis of muscular dystrophies.” Am J Phys Med Rehabil 81(11 Suppl): S175.
Ray, P. N., Belfall, B., Duff, C., et al. (1985). “Cloning of the breakpoint of an X; 21 translocation associated with Duchenne muscular dystrophy.” Nature 318(6047): 672.
Sandri, M., El Meslemani, A. H., Sandri, C., et al. (2001). “Caspase 3 expression correlates with skeletal muscle apoptosis in Duchenne and facioscapulo human muscular dystrophy. A potential target for pharmacological treatment?” J Neuropathol Exp Neurol 60(3): 302.
Spencer, M. J. and Tidball, J. G. (2001). “Do immune cells promote the pathology of dystrophin-deficient myopathies?” Neuromuscul Disord 11(6-7): 556.
St-Pierre, S. J., Chakkalakal, J. V., Kolodziejczyk, S. M., et al. (2004). “Glucocorticoid treatment alleviates dystrophic myofiber pathology by activation of the calcineurin/NF-AT pathway.” Faseb J 18(15): 1937.
Tarnopolsky, M. A., Mahoney, D. J., Vajsar, J., et al. (2004). “Creatine monohydrate enhances strength and body composition in Duchenne muscular dystrophy.” Neurology 62(10): 1771.
Tews, D. S. (2002). “Apoptosis and muscle fibre loss in neuromuscular disorders.” Neuromuscul Disord 12(7-8): 613.
van Essen, A. J., Busch, H. F., to Meerman, G. J., et al. (1992). “Birth and population prevalence of Duchenne muscular dystrophy in The Netherlands.” Hum Genet 88(3): 258.
Wagner, K. R., Hamed, S., Hadley, D. W., et al. (2001). “Gentamicin treatment of Duchenne and Becker muscular dystrophy due to nonsense mutations.” Ann Neurol 49(6): 706.
Walter, M. C., Lochmuller, H., Reilich, P., et al. (2000). “Creatine monohydrate in muscular dystrophies: A double-blind, placebo-controlled clinical study.” Neurology 54(9): 1848.
Yilmaz, O., Karaduman, A. and Topaloglu, H. (2004). “Prednisolone therapy in Duchenne muscular dystrophy prolongs ambulation and prevents scoliosis.” Eur J Neurol 11(8): 541.
Zanardi, M. C., Tagliabue, A., Orcesi, S., et al. (2003). “Body composition and energy expenditure in Duchenne muscular dystrophy.” Eur J Clin Nutr 57(2): 273.
Zatz, M., de Paula, F., Starling, A., et al. (2003). “The 10 autosomal recessive limb-girdle muscular dystrophies.” Neuromuscul Disord 13(7-8): 532.
Zatz, M., Marie, S. K., Cerqueira, A., et al. (1998). “The facioscapulohumeral muscular dystrophy (FSHD1) gene affects males more severely and more frequently than females.” Am J Med Genet 77(2): 155.
Zatz, M., Marie, S. K., Passos-Bueno, M. R., et al. (1995). “High proportion of new mutations and possible anticipation in Brazilian facioscapulohumeral muscular dystrophy families.” Am J Hum Genet 56(1): 99.
Zatz, M., Passos-Bueno, M. R. and Rapaport, D. (1989). “Estimate of the proportion of Duchenne muscular dystrophy with autosomal recessive inheritance.” Am J Med Genet 32(3): 407.
Zhang, R. Z., Sabatelli, P., Pan, T. C., et al. (2002). “Effects on collagen VI mRNA stability and microfibrillar assembly of three COL6A2 mutations in two families with Ullrich congenital muscular dystrophy.” J Biol Chem 277(46): 43557.
Zhao, Y., Haginoya, K., Sun, G., et al. (2003). “Platelet-derived growth factor and its receptors are related to the progression of human muscular dystrophy: an immunohistochemical study.” J Pathol 201(1): 149.
Zubrzycka-Gaarn, E. E., Bulman, D. E., Karpati, G., et al. (1988). “The Duchenne muscular dystrophy gene product is localized in sarcolemma of human skeletal muscle.” Nature 333(6172): 466.
1: Allen D G, Gervasio O L, Yeung E W, Whitehead N P. Calcium and the damage pathways in muscular dystrophy. Can J Physiol Pharmacol. 2010 February; 88(2):83-91.
2: Bushby K, Finkel R, Birnkrant D J, Case L E, Clemens P R, Cripe L, Kaul A, Kinnett K, McDonald C, Pandya S, Poysky J, Shapiro F, Tomersko J, Constantin C; DMD Care Considerations Working Group. Diagnosis and management of Duchenne muscular dystrophy, part 2: implementation of multidisciplinary care. Lancet Neurol. 2010 February; 9(2):177-89. Review. Erratum in: Lancet Neurol. 2010 March; 9(3):237.
3: Bushby K, Finkel R, Birnkrant D J, Case L E, Clemens P R, Cripe L, Kaul A, Kinnett K, McDonald C, Pandya S, Poysky J, Shapiro F, Tomersko J, Constantin C; DMD Care Considerations Working Group. Diagnosis and management of Duchenne muscular dystrophy, part 1: diagnosis, and pharmacological and psychosocial management. Lancet Neurol. 2010 January; 9(1):77-93. Review.
4: Centers for Disease Control and Prevention (CDC). Prevalence of Duchenne/Becker muscular dystrophy among males aged 5-24 years—four states, 2007. MMWR Morb Mortal Wkly Rep. 2009 Oct. 16; 58(40):1119-22.
5: Dooley J, Gordon K E, Dodds L, MacSween J. Duchenne muscular dystrophy: a 30-year population-based incidence study. Clin Pediatr (Phila). 2010 March; 49(2):177-9.
6: Escolar D M, Buyse G, Henricson E, Leshner R, Florence J, Mayhew J, Tesi-Rocha C, Gorni K, Pasquali L, Patel K M, McCarter R, Huang J, Mayhew T, Bertorini T, Carlo J, Connolly A M, Clemens P R, Goemans N, Iannaccone S T, Igarashi M, Nevo Y, Pestronk A, Subramony S H, Vedanarayanan V V, Wessel H; CINRG Group. CINRG randomized controlled trial of creatine and glutamine in Duchenne muscular dystrophy. Ann Neurol. 2005 July; 58(1):151-5.
7: Fisher I, Abraham D, Bouri K, Hoffman E P, Muntoni F, Morgan J. Prednisolone-induced changes in dystrophic skeletal muscle. FASEB J. 2005 May; 19(7):834-6. Epub 2005 Feb. 25. Erratum in: FASEB J. 2005 May; 19(7):1 p following 836. Hoffmann, Eric P [corrected to Hoffman, Eric P].
8: Kinali M, Arechavala-Gomeza V, Feng L, Cirak S, Hunt D, Adkin C, Guglieri M, Ashton E, Abbs S, Nihoyannopoulos P, Garralda M E, Rutherford M, McCulley C, Popplewell L, Graham I R, Dickson G, Wood M J, Wells D J, Wilton S D, Kole R, Straub V, Bushby K, Sewry C, Morgan J E, Muntoni F. Local restoration of dystrophin expression with the morpholino oligomer AVI-4658 in Duchenne muscular dystrophy: a single-blind, placebo-controlled, dose-escalation, proof-of-concept study. Lancet Neurol. 2009 October; 8(10):918-28. Epub 2009 Aug. 25. Erratum in: Lancet Neurol. 2009 December; 8(12):1083.
9: Matthews D J, James K A, Miller L A, Pandya S, Campbell K A, Ciafaloni E, Mathews K D, Miller T M, Cunniff C, Meaney F J, Druschel C M, Romitti P A, Fox D J. Use of Corticosteroids in a Population-Based Cohort of Boys With Duchenne and Becker Muscular Dystrophy. J Child Neurol. 2010 Mar. 5.
10: Millay D P, Goonasekera S A, Sargent M A, Maillet M, Aronow B J, Molkentin J D. Calcium influx is sufficient to induce muscular dystrophy through a TRPC-dependent mechanism. Proc Natl Acad Sci USA. 2009 Nov. 10; 106(45):19023-8. Epub 2009 Oct. 28.
11: van Deutekom J C, Janson A A, Ginjaar I B, Frankhuizen W S, Aartsma-Rus A, Bremmer-Bout M, den Dunnen J T, Koop K, van der Kooi A J, Goemans N M, de Kimpe S J, Ekhart P F, Venneker E H, Platenburg G J, Verschuuren J J, van Ommen G J. Local dystrophin restoration with antisense oligonucleotide PRO051. N Engl J Med. 2007 Dec. 27; 357(26):2677-86.

1. A method of treating muscular dystrophy in a subject, the method comprising administering to a subject suffering from muscular dystrophy a therapeutically effective amount of the Compound (I), or a pharmaceutically acceptable salt thereof, 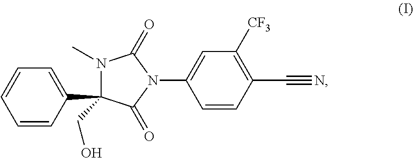 [SMILES: CN1C(=O)N(c2ccc(C#N)c(C(F)(F)F)c2)C(=O)[C@@]1(CO)c1ccccc1]
thereby treating the subject.

2. The method of claim 1, wherein the muscular dystrophy is selected from Duchenne Muscular Dystrophy, Becker Muscular Dystrophy, Emery-Dreifuss Muscular Dystrophy, Limb-Girdle Muscular Dystrophy, Facioscapulohumeral Muscular Dystrophy, Myotonic Dystrophy, Oculopharyngeal Muscular Dystrophy, Distal Muscular Dystrophy, or congenital muscular dystrophy.

3. The method of claim 2, wherein the muscular dystrophy is Duchenne Muscular Dystrophy.

4. The method of claim 1, wherein the method comprises partial or complete alleviation of an awkward manner of walking, stepping, or running; frequent falls; fatigue; difficulty with motor skills; muscle fiber deformities; pseudohypertrophy; skeletal deformities; low endurance; difficulties in standing unaided or inability to ascend staircases; loss of movement; paralysis; cardiomyopathy; development of congestive heart failure; and irregular heartbeat.

5. The method of claim 1, wherein the method improves lifespan.

6. The method of claim 1, wherein the method comprises improving at least one symptom.

7. The method of claim 6, wherein the symptom is fatigue, learning difficulties, intellectual disability, muscle weakness, difficulty with motor skills, difficulty walking, breathing difficulty, heart disease, cardiomyopathy, congestive heart failure, arrhythmia, scoliosis, pseudohypertrophy, muscle wasting, muscle contractures, muscle deformities, and respiratory disorders.

8. The method of claim 1, wherein the Compound (I) or pharmaceutically acceptable salt thereof is administered in multiple doses.

9. The method of claim 1, wherein the Compound (I) or pharmaceutically acceptable salt thereof is administered chronically.

10. The method of claim 9, wherein the Compound (I) or pharmaceutically acceptable salt thereof is administered once daily.

11. The method of claim 9, wherein the Compound (I) or pharmaceutically acceptable salt thereof is administered in a single dose.

12. The method of claim 1, wherein the Compound (I) or a pharmaceutically acceptable salt thereof is administered at a dose of about 0.1 mg to about 1 mg per subject.

13-17. (canceled)

18. The method of claim 12, wherein the dose is from e.g., about 0.2 mg to about 0.8 mg.

19. The method of claim 1, wherein the Compound (I) or a pharmaceutically acceptable salt thereof is administered at a dose of about 2 μg to about 1000 μg per kilogram subject weight.

20-25. (canceled)

26. The method of claim 1, wherein the Compound (I) or a pharmaceutically acceptable salt thereof is administered after meal consumption.

27-29. (canceled)

30. The method of claim 1, wherein the Compound (I) or a pharmaceutically acceptable salt thereof is administered before meal consumption.

31-33. (canceled)

34. The method of claim 1, wherein the compound converts in vivo to the Compound (II), or a pharmaceutically acceptable salt or metabolite thereof,

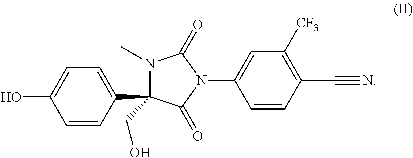 [SMILES: CN1C(=O)N(c2ccc(C#N)c(C(F)(F)F)c2)C(=O)[C@@]1(CO)c1ccc(O)cc1]

35. The method of claim 1, wherein the Compound (I) or pharmaceutically acceptable salt or composition thereof, is administered via oral, subcutaneous, intravenous, intramuscular, intranasal, transdermal, transmucosal, buccal, sublingual, or lung administration.

36. The method of claim 35, wherein the Compound (I) or pharmaceutically acceptable salt or composition thereof, is administered via oral administration.

37. The method of claim 1, wherein the subject is human.

38-40. (canceled)

41. The method of claim 37, wherein the subject is from the age of about 1 year to about 18 years.

42-43. (canceled)

44. The method of claim 1, wherein the compound is in at least 95% enantiomeric excess.

45-46. (canceled)

47. The method of claim 1, wherein the levels of testosterone in the treated subject are not substantially changed as compared to levels of testosterone in the subject before treatment.

48. The method of claim 1, wherein the method of treatment is substantially free of any side effects.

49. The method of claim 1, wherein the compound is characterized by one or both of:
(a) higher activity on muscle and bones of the subject as compared to anabolic steroid treatment; and
(b) lower activity on prostate of the subject as compared to anabolic steroid treatment.

50. A pharmaceutical composition comprising the Compound (I) or a pharmaceutically acceptable salt, metabolite or prodrug thereof, 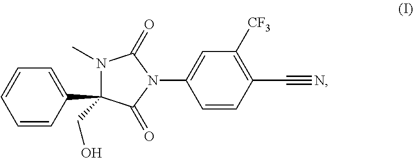 [SMILES: CN1C(=O)N(c2ccc(C#N)c(C(F)(F)F)c2)C(=O)[C@@]1(CO)c1ccccc1]
wherein the pharmaceutical composition comprises about 0.1 mg to about 1 mg of the Compound (I) or a pharmaceutically acceptable salt thereof.

51-66. (canceled)
